SEQUENCE LISTING (286 sequences)

SEQ ID NO: 1 - protein, 271 aa, synthetic construct
  REGION 1..271: note = Synthetic Polypeptide
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 2 - protein, 313 aa, synthetic construct
  REGION 1..313: note = Synthetic Polypeptide
MFTPVRRRVRTAALALSAAAALVLGSTAASGASATPSPAPAPAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 3 - protein, 320 aa, synthetic construct
  REGION 1..320: note = Synthetic Polypeptide
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 4 - protein, 338 aa, synthetic construct
  REGION 1..338: note = Synthetic Polypeptide
MKLSTVLLSAGLASTTLAQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 5 - protein, 1163 aa, synthetic construct
  REGION 1..1163: note = Synthetic Polypeptide
DESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 6 - protein, 1184 aa, synthetic construct
  REGION 1..1184: note = Synthetic Polypeptide
MKFPVPLLFLLQLFFIIATQGDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 7 - protein, 1092 aa, synthetic construct
  REGION 1..1092: note = Synthetic Polypeptide
SSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 8 - protein, 1114 aa, synthetic construct
  REGION 1..1114: note = Synthetic Polypeptide
MVSLRSIFTSSILAAGLTRAHGSSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 9 - protein, 634 aa, synthetic construct
  REGION 1..634: note = Synthetic Polypeptide
EAEAAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSN

SEQ ID NO: 10 - protein, 739 aa, synthetic construct
  REGION 1..739: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLDKREAEAAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 11 - protein, 1493 aa, synthetic construct
  REGION 1..1493: note = Synthetic Polypeptide
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 12 - protein, 1514 aa, synthetic construct
  REGION 1..1514: note = Synthetic Polypeptide
MKFPVPLLFLLQLFFIIATQGAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 13 - protein, 1422 aa, synthetic construct
  REGION 1..1422: note = Synthetic Polypeptide
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSSSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 14 - protein, 1444 aa, synthetic construct
  REGION 1..1444: note = Synthetic Polypeptide
MVSLRSIFTSSILAAGLTRAHGAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSSSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 15 - protein, 1075 aa, synthetic construct
  REGION 1..1075: note = Synthetic Polypeptide
MTIAHHCIFLVILAFLALINVASGATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFSFATVDDSAILSVGGSIAFECCAQEQPPITSTNFTINGIKPWDGSLPDNITGTVYMYAGYYYPLKVVYSNAVSWGTLPISVELPDGTTVSDNFEGYVYSFDDDLSQSNCTIPDPSIHTTSTITTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSLSSSSGQITSSITSSRPIITPFYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHSSSVVSVSETGNTMSLTSSGLSTMSQQPRSTPASSMVGSSTASLEISTYAGSANSLLAGSGLSVFIASLLLAII

SEQ ID NO: 16 - protein, 4 aa, synthetic construct
  REGION 1..4: note = Synthetic Polypeptide
EAEA

SEQ ID NO: 17 - protein, 5 aa, synthetic construct
  REGION 1..5: note = Synthetic Polypeptide
GGGGS

SEQ ID NO: 18 - skipped

SEQ ID NO: 19 - protein, 20 aa, synthetic construct
  REGION 1..20: note = Synthetic Polypeptide
EAAAREAAAREAAAREAAAR

SEQ ID NO: 20 - protein, 59 aa, synthetic construct
  REGION 1..59: note = Synthetic Polypeptide
GSSGSSGSSGSSGSSGSSGSSGSSEAAAREAAAREAAAREAAARGGGGSGGGGSGGGGS

SEQ ID NO: 21 - DNA, 941 bp, synthetic construct
  source 1..941: mol_type = other DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattggatcccga

SEQ ID NO: 22 - DNA, 559 bp, synthetic construct
  source 1..559: mol_type = other DNA
aaataagcatgtttgtttcagatcaaagattagcgtttcaaagttgtggaaaagtgaccatgcaacaatatgcaacacattcggattatctgataagtttcaaagctactaagtaagcccgtttcaagtctccagaccgacatctgccatccagtgattttcttagtcctgaaaaatacgatgtgtaaacataaaccacaaagatcggcctccgaggttgaacccttacgaaagagacatctggtagcgccaatgccaaaaaaaaatcacaccagaaggacaattcccttcccccccagcccattaaagcttaccatttcctattccaatacgttccatagagggcatcgctcggctcattttcgcgtgggtcatactagagcggctagctagtcggctgtttgagctctctaatcgaggggtaaggatgtctaatatgtcataatggctcactatataaagaacccgcttgctcaaccttcgactcctttcccgatcctttgcttgttgcttcttcttttataacaggaaacaaaggaatttatacactttaagaatt

SEQ ID NO: 23 - DNA, 750 bp, synthetic construct
  source 1..750: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatc

SEQ ID NO: 24 - DNA, 1004 bp, synthetic construct
  source 1..1004: mol_type = other DNA
aaatcagccattaatctcacctcagtttttgaatcagtagaattttcaatgaaacaaacggttggtatattatttgatagggtagccaaatttccaaaaatgaacttttcatcaggtaatatcttgaataccgtaatgtagtgactattggaagaaactgctatcaaattatatttcggatagaaatccaaaccccagactgatctcttgagtctcaactctaagtcagccgcgactctaattatctgtggattaggagttagtgtggacaaagcatcagtatagtataactttacggttccattatcagacgctattgcaagaacttcctttccattgatctctccaattcgacagtaattgatatcataaggtaggtctggaaacacactggcgcttgtatcccattctgcaggaatttctggaacggtggtaatggtagttatccaacggagttggggtagttggtatatctggatatgccgcctataggataaaaacaggagagagtgaaccttgcttacggctactagattgttcttgtactcggaattgtcgttatcggaaactagactaatctcatctgtgtgttgcagtactattgagtcgttgtagtatctaccaggagggcattccatgaactagtgagacaaatgagttggattttctcaatagacatatgcaagaatgctacacaacggatgtcgcactctttttcttagttgataatatcatccaatcagaagacacgggctagaaggacttgctcccgaaggataatccactgctactatctcccttcctcacatatagtcttgcagggctcatgcccctttctccttcgaactgcccgatgaggaagtctttagcctatcaaggaattcgggaccatcatcaatttttagagccttacctgatcgcaatcaggatttcactactcatataaatacatcactcaaactccaactttgcttgttcatacaattcttgatattcacaggatc

SEQ ID NO: 25 - DNA, 758 bp, synthetic construct
  source 1..758: mol_type = other DNA
gtgaatttgtcacggaattgaccaagaggtcagacgatcctgtatcccattgagccgttatgctttgtgggggaaaccctatttctatcgtactaagaaaaccaatggtgaactcatattcggtatcaatggcgacgattccagcatagcctgtagacagtaacaacactagggcaacagcaactaacatatcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaagggatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttctagaattcgt

SEQ ID NO: 26 - DNA, 991 bp, synthetic construct
  source 1..991: mol_type = other DNA
cctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaagaattcgcg

SEQ ID NO: 27 - DNA, 500 bp, synthetic construct
  source 1..500: mol_type = other DNA
taatcgaactccgaatgcggttctcctgtaaccttaattgtagcatagatcacttaaataaactcatggcctgacatctgtacacgttcttattggtcttttagcaatcttgaagtctttctattgttccggtcggcattacctaataaattcgaatcgagattgctagtacctgatatcatatgaagtaatcatcacatgcaagttccatgataccctctactaatggaattgaacaaagtttaagcttctcgcacgagaccgaatccatactatgcacccctcaaagttgggattagtcaggaaagctgagcaattaacttccctcgattggcctggacttttcgcttagcctgccgcaatcggtaagtttcattatcccagcggggtgatagcctctgttgctcatcaggccaaaatcatatataagctgtagacccagcacttcaattacttgaaattcaccataacacttgctctagtcaagacttacaattaaa

SEQ ID NO: 28 - DNA, 975 bp, synthetic construct
  source 1..975: mol_type = other DNA
tagcttgggtaggacttgacaagtacggcttccgtggtcataccaaacgcctttgttaccgttggctatacctaatgaccaaggcatttgtggattataacggtatcgtagttgaaaaatatgacgtaaccactggtactagcccccacaaggttgatgctgaatacgggaatcaaggtgccgattttaaaggagtagccactgaagggtttggctgggtcaatgcctcttttattttgggattaacctacttagatgtccaaggcatccgtgcgataggcgccgttacgtcccctgatgtatttttcaggaagctcaaaccttgggaacgcgcaagttatggcctaaggccatgtaacgagatagtcaagtcaaactagaagtatacggtttccccgcagaaatagcagaaataggcgacaaatacatacaacattttcattgtgatagggggcggcggttcctaggagggacaacccccagaaaccttgtagactacgttttcacgacgatgggttattactgtaaaggaagaatatactacccaccagttgaatgtttgaacggatcaaaggtcgaagggagtacacggcccaaccaacgtagctaccggagaaagcaagactttcccaaaccaaatagctccgggtttcttctccggcaacccgtcagtttttgtgtggccggacaaaaattcgcaccctcagtctaattgaaaggtcgggctccgagctctaggcgtttgcgcatgtaatattgcatcccctcccatagataatactgcgcgaacacagggtgcaaattatgatgaccacacatgccagtgaccaaaacagttttttagtctttaaaaaccctcggaacttctgagtatataaaggcttctcatttcctacaagcaaacaaagaagaaacttccactttctaactttttatctatagactttagagttacaaccaacgaacaataacaaa

SEQ ID NO: 29 - DNA, 960 bp, synthetic construct
  source 1..960: mol_type = other DNA
tgaagcttatctgctgagcaagttgtttgaccaaacttgagtcaacagtggttaactatatcctctattattttagatgggagcacatcaagtgtacgggaacaatgcaatcgacaacctgtagcctgacatacatagccatcttgaattgacaaaacttagaatgtcttgaatgtgatagatatgagttcccaaaaatctcttttacgatttcccagttgcggtgtactattacacagaggatatcatagcagacttacaatcctcaggcataaaacgagctttcttatcaaagtgtattcaaatggaccatttgattgcaccaaggcattagccccaaaccataccacacagtaacttgatattctcagcatgcatggaaattccactcataacgcgctattcaccgcgaatacttatctatgaaactgggttctttagtattctttgccaaatttcaccgattagaaattattaggtaatataatttctttggggaaccccttcccgttacgcccgctgcggctttgtggttcttttccagtcttgagcaaattacatctggtctagacagttcttccgtgccccagtatgcgagcgcaaactttcaatcaaacctcgtagcaaattggtacttgaacttcgtatttaaccgctattaaatgtactgactcttacattatgaaaaattttgataaagattttatatttcatctcagttaatctcctaataataatagtctgcataactcaaacggtacttccttttcggaacgcgaagagtagtctctatgtcattctcacactatccgcagcgcaatagagaacgagcatgttacccgactcatcccttgtcgattcggaaacgatttataaatacaattagatcgccaccgatcttcttttgtcaatattataaaaatagtacagattttccttagtcgaatcagatcgcagaaa

SEQ ID NO: 30 - DNA, 876 bp, synthetic construct
  source 1..876: mol_type = other DNA
agatctgagggtgtatacgatgtatcgtgccgaacacatgcacttgacggcacagcaaatggtattcaagaagaccactttagaatgggagttaatagggatggtttcatggaggttaaaacacttcaaggaggcatctgaagcattcaagtatgcactaggtctgaggttttcggtcaaggcatgcaagaaattaattgtattctatctgaacgaacgctccagaatgaaccagccagaaacctcaattgccctcaacaacttaaatcaatccacattatccatccaagagattctcaagtatcgttcgttcctcgatatcaacctaatttcaaacttggtcaaactaggagtttggaatcaccgctggtatgctgagttttctccaaaactcatagaaagccttgcggttgttgtggagaacggagggcttatcaaggtagaaaacgaggttaaggctacctatttcgattcacaagatggagtttacgacttgatgaacgaggtattcaagttcatgaagcattacgattatcctgggactgacaactaagagctcctagtgaagacttgagatggacatgataaacaattatagtgaaaatagaaaccataatacaatattctaatagaggaaccgtttacctgtggttcctattgtggcctactgttactagctagtgtaatacacccttgcctcagctttgcaagttgacaactcagccaaatgatctttgaatgcgcgaaacctcaaggtccatcgaattttctcgaattttcagtgttttcatacagcgtgtcatcttctttcgcgtacttattaaaatcgtacccagatcccttcttcttccttaatttcaattccaacactcaaga

SEQ ID NO: 31 - DNA, 727 bp, synthetic construct
  source 1..727: mol_type = other DNA
agatcttgcaaaatacctttccagctttccagcttcctagcactcatcttgaagatatcaaatattctccattcaaaccaacatcaaaaaatagaataattataatcagtttgaagagcaagagtaattttaaaggaaacacattcatggtcagctagaaggttgactgaagagtcgcaagatatctgagaataaaaaagagcatagctaacaagatgagtaaacacggcaaacagatttaggaacaggtgaagggtttctggctcttcaatgtatatcctgctagccacccattcagaaataacacaaagtaggaccctactgaaaaataaatttaatacatcttcatcctctcattaaaccaccgaccactcaaaccataccagccttgtccaattccatgcatcgtgctatccgtcagaattttcagtgttaatcgaatcggtcattatagctccgtctggggcgacaacttgtcatcacagaatagcacaattatgcgttggaatcgtcaaaaaatcacctccaggtctgtatacatacagaactggttgtaacgacaaccttgtttgattgaggtgactggaaggtggaaagaaagggaggaaataaatattgcaaggaaagaaaaaaaaattgttcacagtcacctcttcaccttcgcgatttcatgtttctttcatgtgctaactgatcccagggcttctccagcgcccttatctgttag

SEQ ID NO: 32 - DNA, 872 bp, synthetic construct
  source 1..872: mol_type = other DNA
ctgcccatctatgactgaatgtggagaagtatcggaacaacccttcactaaggatatctaggctaaactcattcgcgccttagatttctccaaggtatcggttaagtttcctctttcgtactggctaacgatggtgttgctcaacaaagggatggaacggcagctaaagggagtgcatggaatgactttaattggctgagaaagtgttctatttgtccgaatttcttttttctattatctgttcgtttgggcggatctctccagtggggggtaaatggaagatttctgttcatggggtaaggaagctgaaatccttcgtttcttataggggcaagtatactaaatctcggaacattgaatggggtttactttcattggctacagaaattattaagtttgttatggggtgaagttaccagtaattttcattttttcacttcaacttttggggtatttctgtggggtagcatagcttgacaggtaatatgatgtactatgggataggcaagtcttgtgtttcagataccgccaaacgttaaataggaccctcttggtgacttgctaacttagaaagtcatgcccaggtgttacgtaatcttacttggtatgactttttgagtaacggacttgctagagtccttaccagacttccagtttagcaaaccacagattgatctgtcctctggcatatctcaaaccaatcaacacccgtaaccctttcatgaaacaactctagaatgcgtcttatcaacaggattgcccaaaacagtaattggggcggtggaatctacatgggagttccatcgttgtctcggtttttctccctataagctactctggagacgaagtaactaacaccctcaaatatcatt

SEQ ID NO: 33 - DNA, 684 bp, synthetic construct
  source 1..684: mol_type = other DNA
tctgaatccgacctcctctaatctaccactgaagagaagcagtgtattgttcgtctacgtaaatttgaatgtgtaaatggcaaacatggcttcggggatgatttggcatatatattattgtagcatcgtctgtggctctatgagttgtgtggcggatgatgaaaagtttcgtgctgatcccacaatgcggcatttaccaaatggggaaagaccagatttcttcgctgcgccagctagggacagcataatgttccaagaagaagcgattacaggtggattacaaagcgttcgtctgcagttgatgttctacgtgatgggtatgagttgtagtgctacgctccatgaatacttctaatttgtcgttgacaatccatgaataatttaagtttgcttcccaagagtctattgcgaagggtgagccgaatctcttggcgtatgcacccgactcgtcggcttttgtgcgttccttgcaaagctcggtagcaatccgttggtgggagaaatttgtctcacgaatttcagttgggagtagctgttcctggtagcaagttcgaggggatctgtgctcataaaacgtgctcacgccaaaaatattcttacaaaatcttcgcggggtgtttgtcttacataatcgattggatattttcttcaaatttttttttcttactgaagtcccctatagag

SEQ ID NO: 34 - DNA, 842 bp, synthetic construct
  source 1..842: mol_type = other DNA
tcttgccagttgtctcctaagatgtcatcggagtaggctcggctaaagagtagtaatgcatcaagaccaaccaaaacaccttccacgagttcagatgaaccttttaataacttcaggtcactttgatgccggcacaactgggcgagtttcgtatagttaactctgatcttgcactccagaacgggaataggattgactttttgcttccgagaaacgatttgctctctcttcgtctggcttttcactttatatcgcacggaatcaatggatggaactcctaaagctcctaacttcgatgatttgctagccatgactctgtgggacattttcttgcatctcgtttgtaacctgtctgttcctacactaagtttatgagaggctactttggattctagcctcggtggtaaagtgggagataacaacggcataaggcaagaaccagaagtaccataacggtctggtaaagttggtgataacttaattggaagagtgtaagtaagacgtggcttgtaataaggctttccatcaaaaaggttctccgggttggagtttgtgaggctcacatctttgatcagtctttcaatataaattggtaacgttgatgacaatgccggaggtaatttctgtagttgttgatatacgcagataacagattcaaatctccattggttttcatcattgtggcttaaattagatcagaacatggtagtatttaaaaatggatctctttgcagatttactcaatatagcgaaaaaaggagacattcgttacaaaatatgaagataattcgcctcataactcgattaatcaaaacagacggtccagttcttcttttggtagt

SEQ ID NO: 35 - DNA, 689 bp, synthetic construct
  source 1..689: mol_type = other DNA
atctgtactggtactgacaaaggttatccagaatccgagacatttcaacaacagagattccaggcttcaaaacatccattttatcaccaatatctagtaatgcttgcaacaattctggatacttcttctgtgtaaccaaatctcttataaactgaacagctttctgtacgttgtcgtcagtagttggatcaacctcagtggtgacctggcctatcggttttccaaaagacttgtttatcacgtccgaaagctcccatttttgcagatgcgcaactttaaaaggcctggcttgaacatttgcatctcttgttgtgtgttctttgagaaaatattcatcgatctgggtgcttccaacgacagaagatactcttctgagaccagaaagtccccagccatgcttcctaattacaaaatatttgtaggaagatccctgattaggacaaagttgtcttctcatgagttcaactgaaactggggctcaaacggattatgaaaggggtgattaaaggttttcctagccttactttccaaatgtcgaccgagacgaacatttaaaatcctaacatcagaaatttctatccttaatctcattgatggttagtacacttcgcagagtctccacatttgcagaccctcctggataaccaaagcttatctaacagcggcattggacctttgaaaagaccctc

SEQ ID NO: 36 - DNA, 1048 bp, synthetic construct
  source 1..1048: mol_type = other DNA
aaatctgaacacgatgaaacctccccgtagattccaccgccccgttacttttttgggcaatcccgttgataagatccattttagagttgtttctgaaaggattacaggcgttgaagggtcagagagatgccagagaacagaccaattggtagtttgctaaagtggacgtctggcaggtgctctatcgtgttctttatttagggcgttacacttagtaggattacgtaacaatttggcttaaccttctaagttagaaagaaaccaagaggggtcctctttaacgttcagcagtatctaaaacacaaaacctgccctcataatacatcattctatctgtcaagctgtgctaccccacagaaatacccccaagagttaaagtgaaaagaaaagctaaatctgttagacttcaccccataacaaacttgatagttcctgtagccaatgaaagttaaccccattcaatgttccgagatctagtatgcttgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagaattctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaag

SEQ ID NO: 37 - DNA, 758 bp, synthetic construct
  source 1..758: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatcgaattcgt

SEQ ID NO: 38 - DNA, 798 bp, synthetic construct
  source 1..798: mol_type = other DNA
caggtgaacccacctaactatttttaactggcatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgt

SEQ ID NO: 39 - DNA, 1009 bp, synthetic construct
  source 1..1009: mol_type = other DNA
aaataaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacagaatt

SEQ ID NO: 40 - DNA, 686 bp, synthetic construct
  source 1..686: mol_type = other DNA
tgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaatgaattcgcg

SEQ ID NO: 41 - DNA, 477 bp, synthetic construct
  source 1..477: mol_type = other DNA
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

SEQ ID NO: 42 - DNA, 1018 bp, synthetic construct
  source 1..1018: mol_type = other DNA
aaatagcagtttgcggtttcttgatttcatggggggaacaaacaatagtgttgccttaattctaattggcattgttgcttggaatcgaaattgggggataacgtcatatctgaaaagtaaacaacttcgggaaatcaggctgtttgaatggcttggaagcgagatagaaaggggatagcgagatagagggggcggagtagacgaagggtgttaaactgctgaaatctctcaatctggaagaaacggaataaattaactccttgcgataataaaatccgagtccgttatgaccccacaccgtgttgaccacggcataccccatggaatctggtacaaagcgtcagtcttgaagacaccatcacgtgtaggagactgattgtctgaccgtccagcaaaaagggcattataaatcttgctgttaaaggggtgaggggagatgcaggttgttcttttattcgccttgaactttttaattttcccggggttgcggagcgtgaacagttagcccgatctgatagcttgcaagattcaacagtttatccactacaggtcagagagatcgccgcagaagaaatgctcgtctcgtgttccagcacacatactggtgaagtcgttattttgccgaagggggggtaataaggttatgcaccccctctccacaccccagaatcattttttagctgggttcaaggcattagactttgcacatttttcccttaaacacccttgaaacgcggataaacagttgcatgtgcatcctaaaactaggtgagatgcgtactccgtgctccgataataacagtggtgttggggttgctgctagctcacgcactccgttcttttttttcaaccagcaaaattcgatggggagaaacttggggtactttgccgactcctccaccatgctggtatataaataatactcgcccacttttcgtttgctgcttttatatttcatagactgaaaaagactcttcttctactttttcataatatatctcagatatcactactatag

SEQ ID NO: 43 - DNA, 508 bp, synthetic construct
  source 1..508: mol_type = other DNA
gcgatttaaattcgcgaaagaacagcctaataaactccgaagcatgatggcctctatccggaaaacgttaagagatgtggcaacaggagggcacatagaatttttaaagacgctgaagaatgctatcatagtccgtaaaaatgtgatagtactttgtttagtgcgtacgccacttattcggggccaatagctaaacccaggtttgctggcagcaaattcaactgtagattgaatctctctaacaataatggtgttcaatcccctggctggtcacggggaggactatcttgcgtgatccgcttggaaaatgttgtgtatccctttctcaattgcggaaagcatctgctacttcccataggcaccagttacccaattgatatttccaaaaaagattaccatatgttcatctagaagtataaatacaagtggacattcaatgaatatttcattcaattagtcattgacactttcatcaacttactacgtcttattcaacaatgaattcgcg

SEQ ID NO: 44 - DNA, 261 bp, synthetic construct
  source 1..261: mol_type = other DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcg

SEQ ID NO: 45 - DNA, 202 bp, synthetic construct
  source 1..202: mol_type = other DNA
tcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 46 - DNA, 486 bp, synthetic construct
  source 1..486: mol_type = other DNA
attagtgtacatctgataatatagtactaccacgtatgataatgtagagaatagtcttccttgtcgagtgtgtttgcagttttcttgagtttcaaggtttaaatgctggtatattagttcatcgaaggtttcagccaatagcaccttaaatcaatcaaactaattcgactcttacgaaagagcctactgtgtttagtatcgaagtcgtttacctttcatgttgaatagcttcctctctgaccctaacatttcaagatcctcctaaagttacccggattgtgaaattctaatgatccacctgcccaatgcattttttctttattcagtttaccttttttacctaatatacgagcttgttaaagtaagtggcactgcaatactaggcttattgttgatattatgatgaatcgttttcacaaacttgatttcctgtgaactcaccatgtactaaggaaaaaaacatgcatcaccatctgaatatttgac

SEQ ID NO: 47 - DNA, 497 bp, synthetic construct
  source 1..497: mol_type = other DNA
actatgtaactaacgaaacagcatgtactaatagaaccgtatcgagaatatttatttaggtgagtagtaggagtgaaccagacagtcaatttagtgagctgtcccagcttttgtgcattccagaattgccggtcaaattggttatgggttatggggcttttccgattgaggttcagtttctgcggttatctctttcttgacctggtcttttacaggctgttctttctccccatgattattctttagctgaagataccgcttagcctgataatgtcgtcgttttgtaatcaaaatctttagttgggcatcgtctgaggtttcctttggcttctggggttgttagtaggaacgtaggaaccatagtaacttttacacatacattcttatgattgcgaagtaagctgagtctgctgcttggctcccgaagtactttctctttctctaccggttgattctccttctggtgctcctaaacgattgtgttagaagggattgac

SEQ ID NO: 48 - protein, 42 aa, synthetic construct
  REGION 1..42: note = Synthetic Polypeptide
MFTPVRRRVRTAALALSAAAALVLGSTAASGASATPSPAPAP

SEQ ID NO: 49 - protein, 18 aa, synthetic construct
  REGION 1..18: note = Synthetic Polypeptide
MKLSTVLLSAGLASTTLA

SEQ ID NO: 50 - protein, 19 aa, synthetic construct
  REGION 1..19: note = Synthetic Polypeptide
MRFPSIFTAVLFAASSALA

SEQ ID NO: 51 - protein, 22 aa, synthetic construct
  REGION 1..22: note = Synthetic Polypeptide
MVSLRSIFTSSILAAGLTRAHG

SEQ ID NO: 52 - protein, 21 aa, synthetic construct
  REGION 1..21: note = Synthetic Polypeptide
MKFPVPLLFLLQLFFIIATQG

SEQ ID NO: 53 - protein, 18 aa, synthetic construct
  REGION 1..18: note = Synthetic Polypeptide
MQVKSIVNLLLACSLAVA

SEQ ID NO: 54 - protein, 22 aa, synthetic construct
  REGION 1..22: note = Synthetic Polypeptide
MQFNWNIKTVASILSALTLAQA

SEQ ID NO: 55 - protein, 59 aa, synthetic construct
  REGION 1..59: note = Synthetic Polypeptide
MYRNLIIATALTCGAYSAYVPSEPWSTLTPDASLESALKDYSQTFGIAIKSLDADKIKR

SEQ ID NO: 56 - protein, 22 aa, synthetic construct
  REGION 1..22: note = Synthetic Polypeptide
MNLYLITLLFASLCSAITLPKR

SEQ ID NO: 57 - protein, 25 aa, synthetic construct
  REGION 1..25: note = Synthetic Polypeptide
MFEKSKFVVSFLLLLQLFCVLGVHG

SEQ ID NO: 58 - protein, 21 aa, synthetic construct
  REGION 1..21: note = Synthetic Polypeptide
MQFNSVVISQLLLTLASVSMG

SEQ ID NO: 59 - protein, 31 aa, synthetic construct
  REGION 1..31: note = Synthetic Polypeptide
MKSQLIFMALASLVASAPLEHQQQHHKHEKR

SEQ ID NO: 60 - protein, 19 aa, synthetic construct
  REGION 1..19: note = Synthetic Polypeptide
MKFAISTLLIILQAAAVFA

SEQ ID NO: 61 - protein, 23 aa, synthetic construct
  REGION 1..23: note = Synthetic Polypeptide
MKLLNFLLSFVTLFGLLSGSVFA

SEQ ID NO: 62 - protein, 20 aa, synthetic construct
  REGION 1..20: note = Synthetic Polypeptide
MIFNLKTLAAVAISISQVSA

SEQ ID NO: 63 - protein, 40 aa, synthetic construct
  REGION 1..40: note = Synthetic Polypeptide
MKISALTACAVTLAGLAIAAPAPKPEDCTTTVQKRHQHKR

SEQ ID NO: 64 - protein, 18 aa, synthetic construct
  REGION 1..18: note = Synthetic Polypeptide
MSYLKISALLSVLSVALA

SEQ ID NO: 65 - protein, 20 aa, synthetic construct
  REGION 1..20: note = Synthetic Polypeptide
MLSTILNIFILLLFIQASLQ

SEQ ID NO: 66 - protein, 36 aa, synthetic construct
  REGION 1..36: note = Synthetic Polypeptide
MKLSTNLILAIAAASAVVSAAPVAPAEEAANHLHKR

SEQ ID NO: 67 - protein, 19 aa, synthetic construct
  REGION 1..19: note = Synthetic Polypeptide
MFKSLCMLIGSCLLSSVLA

SEQ ID NO: 68 - protein, 19 aa, synthetic construct
  REGION 1..19: note = Synthetic Polypeptide
MKLAALSTIALTILPVALA

SEQ ID NO: 69 - protein, 23 aa, synthetic construct
  REGION 1..23: note = Synthetic Polypeptide
MSFSSNVPQLFLLLVLLTNIVSG

SEQ ID NO: 70 - protein, 46 aa, synthetic construct
  REGION 1..46: note = Synthetic Polypeptide
MQLQYLAVLCALLLNVQSKNVVDFSRFGDAKISPDDTDLESRERKR

SEQ ID NO: 71 - protein, 19 aa, synthetic construct
  REGION 1..19: note = Synthetic Polypeptide
MKIHSLLLWNLFFIPSILG

SEQ ID NO: 72 - protein, 19 aa, synthetic construct
  REGION 1..19: note = Synthetic Polypeptide
MSTLTLLAVLLSLQNSALA

SEQ ID NO: 73 - protein, 38 aa, synthetic construct
  REGION 1..38: note = Synthetic Polypeptide
MINLNSFLILTVTLLSPALALPKNVLEEQQAKDDLAKR

SEQ ID NO: 74 - protein, 16 aa, synthetic construct
  REGION 1..16: note = Synthetic Polypeptide
MFSLAVGALLLTQAFG

SEQ ID NO: 75 - protein, 16 aa, synthetic construct
  REGION 1..16: note = Synthetic Polypeptide
MKILSALLLLFTLAFA

SEQ ID NO: 76 - protein, 20 aa, synthetic construct
  REGION 1..20: note = Synthetic Polypeptide
MKVSTTKFLAVFLLVRLVCA

SEQ ID NO: 77 - protein, 19 aa, synthetic construct
  REGION 1..19: note = Synthetic Polypeptide
MQFGKVLFAISALAVTALG

SEQ ID NO: 78 - protein, 18 aa, synthetic construct
  REGION 1..18: note = Synthetic Polypeptide
MWSLFISGLLIFYPLVLG

SEQ ID NO: 79 - protein, 21 aa, synthetic construct
  REGION 1..21: note = Synthetic Polypeptide
MRNHLNDLVVLFLLLTVAAQA

SEQ ID NO: 80 - protein, 18 aa, synthetic construct
  REGION 1..18: note = Synthetic Polypeptide
MFLKSLLSFASILTLCKA

SEQ ID NO: 81 - protein, 20 aa, synthetic construct
  REGION 1..20: note = Synthetic Polypeptide
MFVFEPVLLAVLVASTCVTA

SEQ ID NO: 82 - protein, 22 aa, synthetic construct
  REGION 1..22: note = Synthetic Polypeptide
MFSPILSLEIILALATLQSVFA

SEQ ID NO: 83 - protein, 16 aa, synthetic construct
  REGION 1..16: note = Synthetic Polypeptide
MIINHLVLTALSIALA

SEQ ID NO: 84 - protein, 19 aa, synthetic construct
  REGION 1..19: note = Synthetic Polypeptide
MLALVRISTLLLLALTASA

SEQ ID NO: 85 - protein, 17 aa, synthetic construct
  REGION 1..17: note = Synthetic Polypeptide
MRPVLSLLLLLASSVLA

SEQ ID NO: 86 - protein, 24 aa, synthetic construct
  REGION 1..24: note = Synthetic Polypeptide
MVLIQNFLPLFAYTLFFNQRAALA

SEQ ID NO: 87 - protein, 20 aa, synthetic construct
  REGION 1..20: note = Synthetic Polypeptide
MVSLTRLLITGIATALQVNA

SEQ ID NO: 88 - protein, 24 aa, synthetic construct
  REGION 1..24: note = Synthetic Polypeptide
MIFDGTTMSIAIGLLSTLGIGAEA

SEQ ID NO: 89 - protein, 31 aa, synthetic construct
  REGION 1..31: note = Synthetic Polypeptide
MVLVGLLTRLVPLVLLAGTVLLLVFVVLSGG

SEQ ID NO: 90 - protein, 16 aa, synthetic construct
  REGION 1..16: note = Synthetic Polypeptide
MLSILSALTLLGLSCA

SEQ ID NO: 91 - protein, 20 aa, synthetic construct
  REGION 1..20: note = Synthetic Polypeptide
MRLLHISLLSIISVLTKANA

SEQ ID NO: 92 - protein, 89 aa, synthetic construct
  REGION 1..89: note = Synthetic Polypeptide
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 93 - protein, 17 aa, synthetic construct
  REGION 1..17: note = Synthetic Polypeptide
MFKSVVYSILAASLANA

SEQ ID NO: 94 - protein, 19 aa, synthetic construct
  REGION 1..19: note = Synthetic Polypeptide
MLLQAFLFLLAGFAAKISA

SEQ ID NO: 95 - protein, 21 aa, synthetic construct
  REGION 1..21: note = Synthetic Polypeptide
MASSNLLSLALFLVLLTHANS

SEQ ID NO: 96 - protein, 29 aa, synthetic construct
  REGION 1..29: note = Synthetic Polypeptide
MNIFYIFLFLLSFVQGLEHTHRRGSLVKR

SEQ ID NO: 97 - protein, 48 aa, synthetic construct
  REGION 1..48: note = Synthetic Polypeptide
MLIIVLLFLATLANSLDCSGDVFFGYTRGDKTDVHKSQALTAVKNIKR

SEQ ID NO: 98 - protein, 49 aa, synthetic construct
  REGION 1..49: note = Synthetic Polypeptide
MESVSSLFNIFSTIMVNYKSLVLALLSVSNLKYARGMPTSERQQGLEER

SEQ ID NO: 99 - protein, 18 aa, synthetic construct
  REGION 1..18: note = Synthetic Polypeptide
MFAFYFLTACISLKGVFG

SEQ ID NO: 100 - protein, 23 aa, synthetic construct
  REGION 1..23: note = Synthetic Polypeptide
MRFSTTLATAATALFFTASQVSA

SEQ ID NO: 101 - protein, 23 aa, synthetic construct
  REGION 1..23: note = Synthetic Polypeptide
MKFAYSLLLPLAGVSASVINYKR

SEQ ID NO: 102 - protein, 22 aa, synthetic construct
  REGION 1..22: note = Synthetic Polypeptide
MKFFAIAALFAAAAVAQPLEDR

SEQ ID NO: 103 - protein, 15 aa, synthetic construct
  REGION 1..15: note = Synthetic Polypeptide
MQFFAVALFATSALA

SEQ ID NO: 104 - protein, 24 aa, synthetic construct
  REGION 1..24: note = Synthetic Polypeptide
MKWVTFISLLFLFSSAYSRGVFRR

SEQ ID NO: 105 - protein, 18 aa, synthetic construct
  REGION 1..18: note = Synthetic Polypeptide
MRSLLILVLCFLPLAALG

SEQ ID NO: 106 - protein, 15 aa, synthetic construct
  REGION 1..15: note = Synthetic Polypeptide
MKVLILACLVALALA

SEQ ID NO: 107 - protein, 18 aa, synthetic construct
  REGION 1..18: note = Synthetic Polypeptide
MFNLKTILISTLASIAVA

SEQ ID NO: 108 - protein, 20 aa, synthetic construct
  REGION 1..20: note = Synthetic Polypeptide
MYRKLAVISAFLATARAQSA

SEQ ID NO: 109 - protein, 85 aa, synthetic construct
  REGION 1..85: note = Synthetic Polypeptide
MRFPSIFTAVLFAASSALAAPVNTTTEDETAQIPAEAVIGYLDLEGDFDVAVLPFSNSTNNGLLFINTTIASIAAKEEGVQLDKR

SEQ ID NO: 110 - protein, 85 aa, synthetic construct
  REGION 1..85: note = Synthetic Polypeptide
MRFPPIFTAALFAASSALAAPANTTTEDETAQIPAEAVIGYLDSEGDSDVAVLPFSNSTNNGLSFINTTIASIAAKEEGVQLDKR

SEQ ID NO: 111 - protein, 85 aa, synthetic construct
  REGION 1..85: note = Synthetic Polypeptide
MRFPSIFTAVLFAASSALAAPANTTTEDETAQIPAEAVISYSDLEGDFDAAALPLSNSTNNGLSSTNTTIASIAAKEEGVQLDKR

SEQ ID NO: 112 - protein, 85 aa, synthetic construct
  REGION 1..85: note = Synthetic Polypeptide
MRPPSIFTAVLFAASSALAAPANTTTEDETTQIPAEAVATYLDLEGDVDVAVLPFSSSTNNGLSFINTTIASIAAKEEGVQLDKR

SEQ ID NO: 113 - protein, 85 aa, synthetic construct
  REGION 1..85: note = Synthetic Polypeptide
MRFPSIFTAALFAASSALAAPANTTTEGETAQTPAEAVIGYRDLEGDFDVAVLPFPNSTNNGLLFTNTTTASIAAKEEGVQLDKR

SEQ ID NO: 114 - protein, 85 aa, synthetic construct
  REGION 1..85: note = Synthetic Polypeptide
MRFPSIFTAVLLAAPSALAAPANATTEDEAAQIPAEAVIGYLDLEGDFDAAVLPFSNSTNNGLLSINTTIASIAAKEEGVQLDKR

SEQ ID NO: 115 - protein, 85 aa, synthetic construct
  REGION 1..85: note = Synthetic Polypeptide
MRFPSIFTAVVFAASSALAAPANTTAEDETAQIPAEAVIGYLGLEGDSDVAALPLSDSTNNGSLSTNTTIASIAAKEEGVQLDKR

SEQ ID NO: 116 - protein, 85 aa, synthetic construct
  REGION 1..85: note = Synthetic Polypeptide
MRLPSIFTAAVFAASSALAAPANTTTEDETAQIPAEAAIGYLDLEGDSDVAVLPLSNSTNNGLLFINTTIASIAAKEEGVQLDKR

SEQ ID NO: 117 - protein, 85 aa, synthetic construct
  REGION 1..85: note = Synthetic Polypeptide
MRFPSIFTAVLFAASSALAAPANTTTEDETAQIPAEAVIGYLDLEGDFDVAVLPFSNSTNDGLSFINTTTASIAAKEEGVQLDKR

SEQ ID NO: 118 - protein, 35 aa, synthetic construct
  REGION 1..35: note = Synthetic Polypeptide
MRFPSIFTAVLFAASSALAAPVNTTTEDETAQIPA

SEQ ID NO: 119 - protein, 37 aa, synthetic construct
  REGION 1..37: note = Synthetic Polypeptide
MLSTILNIFILLLFIQASLQAPIPVVTKYVTEGIAVV

SEQ ID NO: 120 - protein, 36 aa, synthetic construct
  REGION 1..36: note = Synthetic Polypeptide
MSFSSNVPQLFLLLVLLTNIVSGAVISVWSTSKVTK

SEQ ID NO: 121 - protein, 35 aa, synthetic construct
  REGION 1..35: note = Synthetic Polypeptide
MNLYLITLLFASLCSAITLPKRDIIWDYSSEKIMG

SEQ ID NO: 122 - protein, 35 aa, synthetic construct; identical to SEQ ID NO: 118
  REGION 1..35: note = Synthetic Polypeptide

SEQ ID NO: 123 - protein, 35 aa, synthetic construct
  REGION 1..35: note = Synthetic Polypeptide
MLSTILNIFILLLFIQASLQEFDYKDDDDKMVSKG

SEQ ID NO: 124 - protein, 35 aa, synthetic construct
  REGION 1..35: note = Synthetic Polypeptide
MSFSSNVPQLFLLLVLLTNIVSGEFDYKDDDDKMV

SEQ ID NO: 125 - protein, 35 aa, synthetic construct
  REGION 1..35: note = Synthetic Polypeptide
MNLYLITLLFASLCSAEFDYKDDDDKMVSKGEELF

SEQ ID NO: 126 - protein, 35 aa, synthetic construct; identical to SEQ ID NO: 118
  REGION 1..35: note = Synthetic Polypeptide

SEQ ID NO: 127 - protein, 35 aa, synthetic construct
  REGION 1..35: note = Synthetic Polypeptide
MLSTILNIFILLLFIQASLQEFLPSGSDPAFSQPK

SEQ ID NO: 128 - protein, 35 aa, synthetic construct
  REGION 1..35: note = Synthetic Polypeptide
MSFSSNVPQLFLLLVLLTNIVSGEFLPSGSDPAFS

SEQ ID NO: 129 - protein, 35 aa, synthetic construct
  REGION 1..35: note = Synthetic Polypeptide
MNLYLITLLFASLCSAEFLPSGSDPAFSQPKSVLD

SEQ ID NO: 130 - protein, 206 aa, synthetic construct
  REGION 1..206: note = Synthetic Polypeptide
MVAWWSLFLYGLQVAAPALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 131 - protein, 243 aa, synthetic construct
  REGION 1..243: note = Synthetic Polypeptide
MRFPSIFTAVLFAASSALAAPVNTTTEDELEGDFDVAVLPFSASIAAKEEGVSLEKRAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 132 - protein, 205 aa, synthetic construct
  REGION 1..205: note = Synthetic Polypeptide
MRFPSIFTAVLFAASSALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 133 - protein, 212 aa, synthetic construct
  REGION 1..212: note = Synthetic Polypeptide
MLGKNDPMCLVLVLLGLTALLGICQGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 134 - protein, 212 aa, synthetic construct
  REGION 1..212: note = Synthetic Polypeptide
MTKPTQVLVRSVSILFFITLLHLVVAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 135 - protein, 205 aa, synthetic construct
  REGION 1..205: note = Synthetic Polypeptide
MLLQAFLFLLAGFAAKISAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 136 - protein, 204 aa, synthetic construct
  REGION 1..204: note = Synthetic Polypeptide
MKWVTFISLLFLFSSAYSAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 137 - protein, 204 aa, synthetic construct
  REGION 1..204: note = Synthetic Polypeptide
MSFRSLLALSGLVCSGLAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 138 - protein, 202 aa, synthetic construct
  REGION 1..202: note = Synthetic Polypeptide
MKLAYSLLLPLAGVSAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 139 - protein, 247 aa, synthetic construct
  REGION 1..247: note = Synthetic Polypeptide
MRFPSIFTAVLFAASSALAAPVNTTTEDELEGDFDVAVLPFSASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 140 - protein, 24 aa, synthetic construct
  REGION 1..24: note = Synthetic Polypeptide
MAMAGVFVLFSFVLCGFLPDAAFG

SEQ ID NO: 141 - protein, 18 aa, synthetic construct
  REGION 1..18: note = Synthetic Polypeptide
MRSLLILVLCFLPLAALG

SEQ ID NO: 142 - protein, 89 aa, synthetic construct
  REGION 1..89: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLDKREAEA

SEQ ID NO: 143 - protein, 19 aa, synthetic construct
  REGION 1..19: note = Synthetic Polypeptide
MKLILCTVLSLGIAAVCFA

SEQ ID NO: 144 - protein, 19 aa, synthetic construct
  REGION 1..19: note = Synthetic Polypeptide
MKLFVPALLSLGALGLCLA

SEQ ID NO: 145 - protein, 19 aa, synthetic construct
  REGION 1..19: note = Synthetic Polypeptide
MKLFIPALLFLGTLGLCLA

SEQ ID NO: 146 - protein, 23 aa, synthetic construct
  REGION 1..23: note = Synthetic Polypeptide
MESKALLLLALSVWLQSLTVSHG

SEQ ID NO: 147 - protein, 16 aa, synthetic construct
  REGION 1..16: note = Synthetic Polypeptide
MLLIWTLSLLLGAVLG

SEQ ID NO: 148 - protein, 186 aa, synthetic construct
  REGION 1..186: note = Synthetic Polypeptide
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 149 - protein, 186 aa, synthetic construct
  REGION 1..186: note = Synthetic Polypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 150 - protein, 186 aa, synthetic construct
  REGION 1..186: note = Synthetic Polypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 151 - protein, 210 aa, synthetic construct
  REGION 1..210: note = Synthetic Polypeptide
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 152 - protein, 210 aa, synthetic construct
  REGION 1..210: note = Synthetic Polypeptide
MAMAGVFVLFSFVLCGFLPDAVFGAEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 153 - protein, 208 aa, synthetic construct
  REGION 1..208: note = Synthetic Polypeptide
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 154 - protein, 186 aa, synthetic construct
  REGION 1..186: note = Synthetic Polypeptide
AEVDCSRFPNATDKEGKDVLVCNKDLRPICGTDGVTYNNECLLCAYSIEFGTNISKEHDGECKETVPMNCSSYANTTSEDGKVMVLCNRAFNPVCGTDGVTYDNECLLCAHKVEQGASVDKRHDGECRKELAAVSVDCSEYPKPDCTAEDRPLCGSDNKTYGNKCNFCNAVVESNGTLTLSHFGKC

SEQ ID NO: 155 - protein, 208 aa, synthetic construct
  REGION 1..208: note = Synthetic Polypeptide
MAMAGVFVLFSFALCGFLPDAAFGVEVDCSRFPNATNEEGKDVLVCTEDLRPICGTDGVTYSNDCLLCAYNIEYGTNISKEHDGECREAVPVDCSRYPNMTSEEGKVLILCNKAFNPVCGTDGVTYDNECLLCAHNVEQGTSVGKKHDGECRKELAAVDCSEYPKPACTMEYRPLCGSDNKTYDNKCNFCNAVVESNGTLTLSHFGKC

SEQ ID NO: 156 - protein, 239 aa, synthetic construct
  REGION 1..239: note = Synthetic Polypeptide
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVSVDCSEYPKPACTLEYRPLCGSDNKTYGNKCNFCNAVVESNGTLTLSHFGKC

SEQ ID NO: 157 - protein, 185 aa, synthetic construct
  REGION 1..185: note = Synthetic Polypeptide
VEVDCSRFPNTTNEEGKDVLVCTEDLRPICGTDGVTHSECLLCAYNIEYGTNISKEHDGECREAVPMDCSRYPNTTSEEGKVMILCNKALNPVCGTDGVTYDNECVLCAHNLEQGTSVGKKHDGECRKELAAVSVDCSEYPKPACTLEYRPLCGSDNKTYGNKCNFCNAVVESNGTLTLSHFGKC

SEQ ID NO: 158 - protein, 237 aa, synthetic construct
  REGION 1..237: note = Synthetic Polypeptide
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVDCSEYPKPACTLEYRPLCGSDNKTYGNKCNFCNAVVESNGTLTLSHFGKC

SEQ ID NO: 159 - protein, 128 aa, synthetic construct
  REGION 1..128: note = Synthetic Polypeptide
EYGTNISIKHNGECKETVPMDCSRYANMTNEEGKVMMPCDRTYNPVCGTDGVTYDNECQLCAHNVEQGTSVDKKHDGVCGKELAAVSVDCSEYPKPECTAEERPICGSDNKTYGNKCNFCNAVVYVQP

SEQ ID NO: 160 - protein, 195 aa, synthetic construct
  REGION 1..195: note = Synthetic Polypeptide
VDCSRFPNTTNEEGKDVLACTKELHPICGTDGVTYSNECLLCYYNIEYGTNISKEHDGECTEAVPVDCSRYPNTTSEEGKVLIPCNRDFNPVCGSDGVTYENECLLCAHNVEQGTSVGKKHDGGCRKEFAAVSVDCSEYPKPDCTLEYRPLCGSDNKTYASKCNFCNAVVIWEQEKNTRHHASHSVFFISARLVC

SEQ ID NO: 161 - protein, 339 aa, synthetic construct
  REGION 1..339: note = Synthetic Polypeptide
MLPLGLREYGTNTSKEHDGECTEAVPVDCSRYPNTTSEEGKVRILCKKDINPVCGTDGVTYDNECLLCSHSVGQGASIDKKHDGGCRKEFAAVSVDCSEYPKPACMSEYRPLCGSDNKTYVNKCNFCNAVVYVQPWLHSRCRLPPTGTSFLGSEGRETSLLTSRATDLQVAGCTAISAMEATRAAALLGLVLLSSFCELSHLCFSQASCDVYRLSGSRNLACPRIFQPVCGTDNVTYPNECSLCRQMLRSRAVYKKHDGRCVKVDCTGYMRATGGLGTACSQQYSPLYATNGVIYSNKCTFCSAVANGEDIDLLAVKYPEEESWISVSPTPWRMLSAGA

SEQ ID NO: 162 - protein, 238 aa, synthetic construct
  REGION 1..238: note = Synthetic Polypeptide
MSWWGIKPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 163 - protein, 284 aa, synthetic construct
  REGION 1..284: note = Synthetic Polypeptide
MSSQNQLHRRRRPLPGGQDLNKYYWPHCTSDRFSWLLHVTAEQFRHCVCIYLQPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 164 - protein, 186 aa, synthetic construct
  REGION 1..186: note = Synthetic Polypeptide
VEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNVVQGTSVGKKHDGECRKELAAVSVDCSEYPKPACPKDYRPVCGSDNKTYSNKCNFCNAVVESNGTLTLNHFGKC

SEQ ID NO: 165 - protein, 210 aa, synthetic construct
  REGION 1..210: note = Synthetic Polypeptide
MAMAGVFLLFSFALCGFLPDAAFGVEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNIVQGTSVGKKHDGECRKELAAVSVDCSEYPKPACPKDYRPVCGSDNKTYSNKCNFCNAVVESNGTLTLNHFGKC

SEQ ID NO: 166 - protein, 205 aa, synthetic construct
  REGION 1..205: note = Synthetic Polypeptide
MAGVFVLLSLVLCCFPDAAFGVEVDCSRFPNTTNEEGKDVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 167 - protein, 171 aa, synthetic construct
  REGION 1..171: note = Synthetic Polypeptide
QVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVSVDCSGYPKPACTMEYMPLCGSDNKTYGNKCNFCNAVV

SEQ ID NO: 168 - protein, 207 aa, synthetic construct
  REGION 1..207: note = Synthetic Polypeptide
MTMPGAFVVLSFVLCCFPDATFGVEVDCSTYPNTTNEEGKEVLVCSKILSPICGTDGVTYSNECLLCANNIEYGTNISKYHDGECKEFVPVNCSRYPNTTNEEGKVMLICNKDLSPVCGTDGVTYDNECLLCAHNLEPGTSVGKKYDGECKKEIATVDCSDYPKPVCSLESMPLCGSDNKTYSNKCNFCNAVVDSNETLTLSHFGKC

SEQ ID NO: 169 - protein, 207 aa, synthetic construct
  REGION 1..207: note = Synthetic Polypeptide
MTMAGVFVLLSFALCCFPDAAFGVEVDCSTYPNTTNEEGKEVLVCTKILSPICGTDGVTYSNECLLCAYNIEYGTNVSKDHDGECKEVVPVDCSRYPNSTNEEGKVVMLCSKDLNPVCGTDGVTYDNECVLCAHNVESGTSVGKKYDGECKKETATVDCSDYPKPACTLEYMPFCGSDSKTYSNKCNFCNAVVDSNGTLTLSHFGKC

SEQ ID NO: 170 - protein, 207 aa, synthetic construct
  REGION 1..207: note = Synthetic Polypeptide
MTTAGVFVLLSFALCSFPDAAFGVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 171 - protein, 207 aa, synthetic construct
  REGION 1..207: note = Synthetic Polypeptide
MTTAGVFVLLSFTLCSFPDAAFGVEVDCSPYPNTTNEEGKEVLVCNKILSPICGTDGVTYSNECLLCAYNLEYGTNISKDYDGECKEVAPVDCSRHPNTTNEEGKVELLCNKDLNPICGTNGVTYDNECLLCARNLESGTSIGKKYDGECKKEIATVDCSDYPKPVCTLEEMPLCGSDNKTYGNKCNFCNAVVDSNGTLTLSHFGKC

SEQ ID NO: 172 - protein, 207 aa, synthetic construct
  REGION 1..207: note = Synthetic Polypeptide
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVVDSNGTLTLSRFGKC

SEQ ID NO: 173 - protein, 207 aa, synthetic construct
  REGION 1..207: note = Synthetic Polypeptide
MTMTGVFVLLSFAICCFPDAAFGVEVDCSTYPNTTNEEGKEVLVCTKILSPICGTDGVTYSNECLLCAYNIEYGTNVSKDHDGECKEVVPVDCSKYPNTTNEEGKVVLLCSKDLSPVCGTDGVTYDNECLLCARNLEPGSSVGKKYDGECKKEIATIDCSDYPKPVCSLEYMPLCGSDSKTYDNKCNFCNAVVDSNGTLTLSHFGKC

SEQ ID NO: 174 - protein, 207 aa, synthetic construct
  REGION 1..207: note = Synthetic Polypeptide
MTTAGVFVLLSFVLCCFPDAVFGVEVDCSTYPNTTNEEGKEVLVCTKILSPICGTDGVTYSNECLLCAYNIEYGTNVSKDHDGECKEVVPVNCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 175 - protein, 207 aa, synthetic construct
  REGION 1..207: note = Synthetic Polypeptide
MTTAGVFVLLSIALCCFPDAAFGVEVDCSAYSNTTSEEGKEVLSCTKILSPICGTDGVTYSNECLLCAYNIEYGTNISKDHDGECKEVVSVDCSRYPNTTNEEGKAVLLCNKDLSPVCGTDGVTYDNECLLCAHNLEPGTSVGKKYDGACKKEIATVDCSDYPKPVCTLEYLPLCGSDSKTYSNKCDFCNAVVDSNGTLTLSHFGKC

SEQ ID NO: 176 - protein, 206 aa, synthetic construct
  REGION 1..206: note = Synthetic Polypeptide
MTTAGVFVLLSFALCCFPDAAFGVEVDCSTYPNTTNEEGKEVLVCTKILSPICGTDGTTYSNECLLCAYNIEYGTNVSKDHDGECKVVPVDCSKYPNTTNEDGKVVLLCNKALSPICGTDRVTYDNECLMCAHNLEPGTSVGKKHDGECQKEVATVDCSDYPKPVCSLEYMPLCGSDGKTYSNKCNFCNAVVNSNGTLTLSHFEKC

SEQ ID NO: 177 - protein, 213 aa, synthetic construct
  REGION 1..213: note = Synthetic Polypeptide
MTTAGVFVLLSFVLCCFFPDAAFGVEVDCSTYPNTTNEEGKEVLVCAKILSPVCGTDGVTYSNECLLCAHNIENGTNVGKDHDGKCKEAVPVDCSRYPNTTDEEGKVVLLCNKDVSPVCGTDGVTYDNECLLCAHNLEAGTSVDKKNDSECKTEDTTLAAVSVDCSDYPKPVCTLEYLPLCGSDNKTYSNKCRFCNAVVDSNGTLTLSRFGKC

SEQ ID NO: 178 - protein, 207 aa, synthetic construct
  REGION 1..207: note = Synthetic Polypeptide
MTTAGVFVLLSFALCCSPDAAFGVEVDCSTYPNTTNEEGKEVLACTKILSPICGTDGVTYSNECLLCAYNMEYGTNVSKDHDGKCKEVVPVDCSRYPNTTNEEGKVVLLCNKDLSPVCGTDGVTYDNECLLCARNLEPGASVGKKYDGECKKEIATVDCSDYPKPVCSLEHMPLCGSDSKTYSNKCTFCNAVVDSNGTLTLSHFGKC

SEQ ID NO: 179 - protein, 207 aa, synthetic construct
  REGION 1..207: note = Synthetic Polypeptide
MTTAGVFVLLSFALCCFPDAAFGVEVDCSTYPNTTNEEGREVLVCTKILSPICGTDGVTYSNECLLCAYNIEYGTNVSKDHDGECKEVAPVGCSRYPNTTNEEGKVVLLCNKDLSPVCGTDGVTYDNECLLCARHLEPGTSVGKKYDGECKKEIATVDCSDYPKPVCSLEYMPLCGSDSKTYSNKCNFCNAVLDSNGTLTLSHFGKC

SEQ ID NO: 180 - protein, 207 aa, synthetic construct
  REGION 1..207: note = Synthetic Polypeptide
MTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 181 - protein, 207 aa, synthetic construct
  REGION 1..207: note = Synthetic Polypeptide
MTTAGVFVLLSFVLCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 182 - protein, 282 aa, synthetic construct
  REGION 1..282: note = Synthetic Polypeptide
MSSQNQLPSRCRPLPGSQDLNKYYQPHCTGDRFCWLFYVTVEQFRHCICIYLQLALERPSHEQSGQPADSRNTSTMTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 183 - protein, 193 aa, synthetic construct
  REGION 1..193: note = Synthetic Polypeptide
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVV

SEQ ID NO: 184 - protein, 190 aa, synthetic construct
  REGION 1..190: note = Synthetic Polypeptide
EAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 185 - protein, 199 aa, synthetic construct
  REGION 1..199: note = Synthetic Polypeptide
EEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 186 - protein, 275 aa, synthetic construct
  REGION 1..275: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 187 - protein, 273 aa, synthetic construct
  REGION 1..273: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 188 - protein, 188 aa, synthetic construct
  REGION 1..188: note = Synthetic Polypeptide
EAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 189 - protein, 206 aa, synthetic construct
  REGION 1..206: note = Synthetic Polypeptide
MTMAGVFVLLSFILCCFPDTAFGVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 190 - protein, 272 aa, synthetic construct
  REGION 1..272: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLDKREAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 191 - protein, 187 aa, synthetic construct
  REGION 1..187: note = Synthetic Polypeptide
EAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 192 - protein, 402 aa, synthetic construct
  REGION 1..402: note = Synthetic Polypeptide
MFFYNTDFRMGSISAANAEFCFDVFNELKVQHTNENILYSPLSIIVALAMVYMGARGNTEYQMEKALHFDSIAGLGGSTQTKVQKPKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 193 - protein, 388 aa, synthetic construct
  REGION 1..388: note = Synthetic Polypeptide
MDSISVTNAKFCFDVFNEMKVHHVNENILYCPLSILTALAMVYLGARGNTESQMKKVLHFDSITGAGSTTDSQCGSSEYVHNLFKELLSEITRPNATYSLEIADKLYVDKTFSVLPEYLSCARKFYTGGVEEVNFKTAAEEARQLINSWVEKETNGQIKDLLVSSSIDFGTTMVFINTIYFKGIWKIAFNTEDTREMPFSMTKEESKPVQMMCMNNSFNVATLPAEKMKILELPYASGDLSMLVLLPDEVSGLERIEKTINFDKLREWTSTNAMAKKSMKVYLPRMKIEEKYNLTSILMALGMTDLFSRSANLTGISSVDNLMISDAVHGVFMEVNEEGTEATGSTGAIGNIKHSLELEEFRADHPFLFFIRYNPTNAILFFGRYWSP

SEQ ID NO: 194 - protein, 386 aa, synthetic construct
  REGION 1..386: note = Synthetic Polypeptide
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 195 - protein, 474 aa, synthetic construct
  REGION 1..474: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 196 - protein, 389 aa, synthetic construct
  REGION 1..389: note = Synthetic Polypeptide
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 197 - protein, 419 aa, synthetic construct
  REGION 1..419: note = Synthetic Polypeptide
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 198 - protein, 406 aa, synthetic construct
  REGION 1..406: note = Synthetic Polypeptide
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 199 - protein, 451 aa, synthetic construct
  REGION 1..451: note = Synthetic Polypeptide
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 200 - protein, 386 aa, synthetic construct
  REGION 1..386: note = Synthetic Polypeptide
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 201 - protein, 386 aa, synthetic construct
  REGION 1..386: note = Synthetic Polypeptide
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 202 - protein, 417 aa, synthetic construct
  REGION 1..417: note = Synthetic Polypeptide
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 203 - protein, 383 aa, synthetic construct
  REGION 1..383: note = Synthetic Polypeptide
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 204 - protein, 386 aa, synthetic construct
  REGION 1..386: note = Synthetic Polypeptide
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 205 - protein, 411 aa, synthetic construct
  REGION 1..411: note = Synthetic Polypeptide
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 206 - protein, 383 aa, synthetic construct
  REGION 1..383: note = Synthetic Polypeptide
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 207 - protein, 408 aa, synthetic construct
  REGION 1..408: note = Synthetic Polypeptide
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 208 - protein, 383 aa, synthetic construct
  REGION 1..383: note = Synthetic Polypeptide
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 209 - protein, 386 aa, synthetic construct
  REGION 1..386: note = Synthetic Polypeptide
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 210 - protein, 386 aa, synthetic construct
  REGION 1..386: note = Synthetic Polypeptide
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 211 - protein, 386 aa, synthetic construct
  REGION 1..386: note = Synthetic Polypeptide
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 212 - protein, 386 aa, synthetic construct
  REGION 1..386: note = Synthetic Polypeptide
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 213 - protein, 386 aa, synthetic construct
  REGION 1..386: note = Synthetic Polypeptide
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 214 - protein, 386 aa, synthetic construct
  REGION 1..386: note = Synthetic Polypeptide
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 215 - protein, 386 aa, synthetic construct
  REGION 1..386: note = Synthetic Polypeptide
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 216 - protein, 386 aa, synthetic construct
  REGION 1..386: note = Synthetic Polypeptide
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 217 - protein, 386 aa, synthetic construct
  REGION 1..386: note = Synthetic Polypeptide
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 218 - protein, 386 aa, synthetic construct
  REGION 1..386: note = Synthetic Polypeptide
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 219 - protein, 386 aa, synthetic construct
  REGION 1..386: note = Synthetic Polypeptide
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 220 - protein, 386 aa, synthetic construct
  REGION 1..386: note = Synthetic Polypeptide
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 221 - protein, 391 aa, synthetic construct
  REGION 1..391: note = Synthetic Polypeptide
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 222 - protein, 386 aa, synthetic construct
  REGION 1..386: note = Synthetic Polypeptide
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 223 - protein, 386 aa, synthetic construct
  REGION 1..386: note = Synthetic Polypeptide
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 224 - protein, 386 aa, synthetic construct
  REGION 1..386: note = Synthetic Polypeptide
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 225 - protein, 386 aa, synthetic construct
  REGION 1..386: note = Synthetic Polypeptide
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 226 - protein, 385 aa, synthetic construct
  REGION 1..385: note = Synthetic Polypeptide
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 227 - protein, 386 aa, synthetic construct
  REGION 1..386: note = Synthetic Polypeptide
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 228 - protein, 386 aa, synthetic construct
  REGION 1..386: note = Synthetic Polypeptide
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 229 - protein, 386 aa, synthetic construct
  REGION 1..386: note = Synthetic Polypeptide
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 230 - protein, 385 aa, synthetic construct
  REGION 1..385: note = Synthetic Polypeptide
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 231 - protein, 386 aa, synthetic construct
  REGION 1..386: note = Synthetic Polypeptide
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 232 - protein, 386 aa, synthetic construct
  REGION 1..386: note = Synthetic Polypeptide
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 233 - protein, 386 aa, synthetic construct
  REGION 1..386: note = Synthetic Polypeptide
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 234 - protein, 386 aa, synthetic construct
  REGION 1..386: note = Synthetic Polypeptide
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 235 - protein, 386 aa, synthetic construct
  REGION 1..386: note = Synthetic Polypeptide
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 236 - protein, 386 aa, synthetic construct
  REGION 1..386: note = Synthetic Polypeptide
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 237 - protein, 386 aa, synthetic construct
  REGION 1..386: note = Synthetic Polypeptide
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 238 - protein, 386 aa, synthetic construct
  REGION 1..386: note = Synthetic Polypeptide
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 239 - protein, 386 aa, synthetic construct
  REGION 1..386: note = Synthetic Polypeptide
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 240 - protein, 381 aa, synthetic construct
  REGION 1..381: note = Synthetic Polypeptide
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 241 - protein, 391 aa, synthetic construct
  REGION 1..391: note = Synthetic Polypeptide
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 242 - protein, 383 aa, synthetic construct
  REGION 1..383: note = Synthetic Polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 243 - protein, 386 aa, synthetic construct
  REGION 1..386: note = Synthetic Polypeptide
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 244 - protein, 392 aa, synthetic construct
  REGION 1..392: note = Synthetic Polypeptide
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 245 - protein, 386 aa, synthetic construct
  REGION 1..386: note = Synthetic Polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 246 - protein, 395 aa, synthetic construct
  REGION 1..395: note = Synthetic Polypeptide
  VARIANT 99: note = misc_feature - Xaa can be any naturally occurring amino acid
  VARIANT 233: note = misc_feature - Xaa can be any naturally occurring amino acid
  VARIANT 341: note = misc_feature - Xaa can be any naturally occurring amino acid
  VARIANT 379: note = misc_feature - Xaa can be any naturally occurring amino acid
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 247 - protein, 386 aa, synthetic construct
  REGION 1..386: note = Synthetic Polypeptide
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 248 - protein, 386 aa, synthetic construct
  REGION 1..386: note = Synthetic Polypeptide
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 249 - protein, 385 aa, synthetic construct
  REGION 1..385: note = Synthetic Polypeptide
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 250 - protein, 386 aa, synthetic construct
  REGION 1..386: note = Synthetic Polypeptide
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 251 - protein, 383 aa, synthetic construct
  REGION 1..383: note = Synthetic Polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 252 - protein, 386 aa, synthetic construct
  REGION 1..386: note = Synthetic Polypeptide
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 253 - protein, 386 aa, synthetic construct
  REGION 1..386: note = Synthetic Polypeptide
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 254 - protein, 2174 aa, synthetic construct
  REGION 1..2174: note = Synthetic Polypeptide
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 255 - protein, 386 aa, synthetic construct
  REGION 1..386: note = Synthetic Polypeptide
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 256 - protein, 2104 aa, synthetic construct
  REGION 1..2104: note = Synthetic Polypeptide
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 257 - protein, 386 aa, synthetic construct
  REGION 1..386: note = Synthetic Polypeptide
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 258 - protein, 420 aa, synthetic construct
  REGION 1..420: note = Synthetic Polypeptide
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 259 - protein, 386 aa, synthetic construct
  REGION 1..386: note = Synthetic Polypeptide
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 260 - protein, 386 aa, synthetic construct
  REGION 1..386: note = Synthetic Polypeptide
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 261 - protein, 386 aa, synthetic construct
  REGION 1..386: note = Synthetic Polypeptide
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 262 - protein, 386 aa, synthetic construct
  REGION 1..386: note = Synthetic Polypeptide
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 263 - protein, 386 aa, synthetic construct
  REGION 1..386: note = Synthetic Polypeptide
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 264 - protein, 399 aa, synthetic construct
  REGION 1..399: note = Synthetic Polypeptide
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 265 - protein, 389 aa, synthetic construct
  REGION 1..389: note = Synthetic Polypeptide
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 266 - protein, 474 aa, synthetic construct
  REGION 1..474: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 267 - protein, 389 aa, synthetic construct
  REGION 1..389: note = Synthetic Polypeptide
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 268 - protein, 474 aa, synthetic construct
  REGION 1..474: note = Synthetic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 269 - protein, 426 aa, synthetic construct
  REGION 1..426: note = Synthetic Polypeptide
TRAPDCGGILTPLGLSYLAEVSKPHAEVVLRQDLMAQRASDLFLGSMEPSRNRITSVKVADLWLSVIPEAGLRLGIEVELRIAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVNVVVHKDYVLVPCKLKLRSTMA

SEQ ID NO: 270 - protein, 388 aa, synthetic construct; identical to SEQ ID NO: 193
  REGION 1..388: note = Synthetic Polypeptide

SEQ ID NO: 271 - protein, 1185 aa, synthetic construct
  REGION 1..1185: note = Synthetic Polypeptide
CSTWGGGHFSTFDKYQYDFTGTCNYIFATVCDESSPDFNIQFRRGLDKKIARIIIELGPSVIIVEKDSISVRSVGVIKLPYASNGIQIAPYGRSVRLVAKLMEMELVVMWNNEDYLMVLTEKKYMGKTCGMCGNYDGYELNDFVSEGKLLDTYKFAALQKMDDPSEICLSEEISIPAIPHKKYAVICSQLLNLVSPTCSVPKDGFVTRCQLDMQDCSEPGQKNCTCSTLSEYSRQCAMSHQVVFNWRTENFCSVGKCSANQIYEECGSPCIKTCSNPEYSCSSHCTYGCFCPEGTVLDDISKNRTCVHLEQCPCTLNGETYAPGDTMKAACRTCKCTMGQWNCKELPCPGRCSLEGGSFVTTFDSRSYRFHGVCTYILMKSSSLPHNGTLMAIYEKSGYSHSETSLSAIIYLSTKDKIVISQNELLTDDDELKRLPYKSGDITIFKQSSMFIQMHTEFGLELVVQTSPVFQAYVKVSAQFQGRTLGLCGNYNGDTTDDFMTSMDITEGTASLFVDSWRAGNCLPAMERETDPCALSQLNKISAETHCSILTKKGTVFETCHAVVNPTPFYKRCVYQACNYEETFPYICSALGSYARTCSSMGLILENWRNSMDNCTITCTGNQTFSYNTQACERTCLSLSNPTLECHPTDIPIEGCNCPKGMYLNHKNECVRKSHCPCYLEDRKYILPDQSTMTGGITCYCVNGRLSCTGKLQNPAESCKAPKKYISCSDSLENKYGATCAPTCQMLATGIECIPTKCESGCVCADGLYENLDGRCVPPEECPCEYGGLSYGKGEQIQTECEICTCRKGKWKCVQKSRCSSTCNLYGEGHITTFDGQRFVFDGNCEYILAMDGCNVNRPLSSFKIVTENVICGKSGVTCSRSISIYLGNLTIILRDETYSISGKNLQVKYNVKKNALHLMFDIIIPGKYNMTLIWNKHMNFFIKISRETQETICGLCGNYNGNMKDDFETRSKYVASNELEFVNSWKENPLCGDVYFVVDPCSKNPYRKAWAEKTCSIINSQVFSACHNKVNRMPYYEACVRDSCGCDIGGDCECMCDAIAVYAMACLDKGICIDWRTPEFCPVYCEYYNSHRKTGSGGAYSYGSSVNCTWHYRPCNCPNQYYKYVNIEGCYNCSHDEYFDYEKEKCMPCAMQPTSVTLPTATQPTSPSTSSASTVLTETTNPPV

SEQ ID NO: 272 - protein, 129 aa, synthetic construct [sequence omitted]

SEQ ID NO: 273 - protein, 129 aa, synthetic construct
  REGION 1..129: note = Synthetic Polypeptide
KVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 274 - protein, 130 aa, synthetic construct
  REGION 1..130: note = Synthetic Polypeptide
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 275 - protein, 129 aa, synthetic construct
  REGION 1..129: note = Synthetic Polypeptide
KVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 276 - protein, 449 aa, synthetic construct
  REGION 1..449: note = Synthetic Polypeptide
IEVNCSLYASGIGKDGTSWVACPRNLKPVCGTDGSTYSNECGICLYNREHGANVEKEYDGECRPKHVMIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 277 - protein, 139 aa, synthetic construct
  REGION 1..139: note = Synthetic Polypeptide
MAGARGCVVLLAAALMLVGAVLGSEDRSRLLGAPVPVDENDEGLQRALQFAMAEYNRASNDKYSSRVVRVISAKRQLVSGIKYILQVEIGRTTCPKSSGDLQSCEFHDEPEMAKYTTCTFVVYSIPWLNQIKLLESKCQ

SEQ ID NO: 278 - protein, 450 aa, synthetic construct
  REGION 1..450: note = Synthetic Polypeptide
SEVCFPRLGCFSDDAPWAGIVQRPLKILPWSPKDVDTRFLLYTNQNQNNYQELVADPSTITNSNFRMDRKTRFIIHGFIDKGEEDWLSNICKNLFKVESVNCICVDWKGGSRTGYTQASQNIRIVGAEVAYFVEVLKSSLGYSPSNVHVIGHSLGSHAAGEAGRRTNGTIERITGLDPAEPCFQGTPELVRLDPSDAKFVDVIHTDAAPIIPNLGFGMSQTVGHLDFFPNGGKQMPGCQKNILSQIVDIDGIWEGTRDFVACNHLRSYKYYADSILNPDGFAGFPCDSYNVFTANKCFPCPSEGCPQMGHYADRFPGKTNGVSQVFYLNTGDASNFARWRYKVSVTLSGKKVTGHILVSLFGNEGNSRQYEIYKGTLQPDNTHSDEFDSDVEVGDLQKVKFIWYNNNVINPTLPRVGASKITVERNDGKVYDFCSQETVREEVLLTLNPC

SEQ ID NO: 279 - protein, 455 aa, synthetic construct
  REGION 1..455: note = Synthetic Polypeptide
GLVAADRITGGKDFRDIESKFALRTPEDTAEDTCHLIPGVTESVANCHFNHSSKTFVVIHGWTVTGMYESWVPKLVAALYKREPDSNVIVVDWLSRAQQHYPVSAGYTKLVGQDVAKFMNWMADEFNYPLGNVHLLGYSLGAHAAGIAGSLTSKKVNRITGLDPAGPNFEYAEAPSRLSPDDADFVDVLHTFTRGSPGRSIGIQKPVGHVDIYPNGGTFQPGCNIGEALRVIAERGLGDVDQLVKCSHERSVHLFIDSLLNEENPSKAYRCNSKEAFEKGLCLSCRKNRCNNMGYEINKVRAKRSSKMYLKTRSQMPYKVFHYQVKIHFSGTESNTYTNQAFEISLYGTVAESENIPFTLPEVSTNKTYSFLLYTEVDIGELLMLKLKWISDSYFSWSNWWSSPGFDIGKIRVKAGETQKKVIFCSREKMSYLQKGKSPVIFVKCHDKSLNRKSG

SEQ ID NO: 280 - protein, 685 aa, synthetic construct
  REGION 1..685: note = Synthetic Polypeptide
APKKGVRWCVISTAEYSKCRQWQSKIRRTNPMFCIRRASPTDCIRAIAAKRADAVTLDGGLVFEADQYKLRPVAAEIYGTEENPQTYYYAVAVVKKGFNFQLNQLQGRKSCHTGLGRSAGWNIPIGLLRRFLDWAGPPEPLQKAVAKFFSQSCVPCADGNAYPNLCQLCIGKGKDKCACSSQEPYFGYSGAFNCLHKGIGDVAFVKESTVFENLPQKADRDKYELLCPDNTRKPVEAFRECHLARVPSHAVVARSVNGKENSIWELLYQSQKKFGKSNPQEFQLFGSPGQQKDLLFRDATIGFLKIPSKIDSKLYLGLPYLTAIQGLRETAAEVEARQAKVVWCAVGPEELRKCRQWSSQSSQNLNCSLASTTEDCIVQVLKGEADAMSLDGGFIYTAGKCGLVPVLAENQKSRQSSSSDCVHRPTQGYFAVAVVRKANGGITWNSVRGTKSCHTAVDRTAGWNIPMGLLVNQTGSCKFDEFFSQSCAPGSQPGSNLCALCVGNDQGVDKCVPNSNERYYGYTGAFRCLAENAGDVAFVKDVTVLDNTNGQNTEEWARELRSDDFELLCLDGTRKPVTEAQNCHLAVAPSHAVVSRKEKAAQVEQVLLTEQAQFGRYGKDCPDKFCLFRSETKNLLFNDNTEVLAQLQGKTTYEKYLGSEYVTAIANLKQCSVSPLLEACAFMMR

SEQ ID NO: 281 - protein, 689 aa, synthetic construct
  REGION 1..689: note = Synthetic Polypeptide
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 282 - protein, 66 aa, synthetic construct
  REGION 1..66: note = Synthetic Polypeptide
APVNTTTEDETAQIPAEAVIGYSDLEGDFDVAVLPFSNSTNNGLLFINTTIASIAAKEEGVSLDKR

SEQ ID NO: 283 - protein, 70 aa, synthetic construct
  REGION 1..70: note = Synthetic Polypeptide
APVNTTTEDETAQIPAEAVIGYSDLEGDFDVAVLPFSNSTNNGLLFINTTIASIAAKEEGVSLDKREAEA

SEQ ID NO: 284 - protein, 1405 aa, synthetic construct
  REGION 1..1405: note = Synthetic Polypeptide
MTIAHHCIFLVILAFLALINVASGAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFSFATVDDSAILSVGGSIAFECCAQEQPPITSTNFTINGIKPWDGSLPDNITGTVYMYAGYYYPLKVVYSNAVSWGTLPISVELPDGTTVSDNFEGYVYSFDDDLSQSNCTIPDPSIHTTSTITTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSLSSSSGQITSSITSSRPIITPFYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHSSSVVSVSETGNTMSLTSSGLSTMSQQPRSTPASSMVGSSTASLEISTYAGSANSLLAGSGLSVFIASLLLAII

SEQ ID NO: 285 - protein, 24 aa, synthetic construct
  REGION 1..24: note = Synthetic Polypeptide
GSSGSSGSSGSSGSSGSSGSSGSS

SEQ ID NO: 286 - protein, 15 aa, synthetic construct
  REGION 1..15: note = Synthetic Polypeptide
GGGGSGGGGSGGGGS

(19) United States
(12) Patent Application Publication
(10) Pub. No.: US 2024/0026325 A1
(43) Pub. Date: Jan. 25, 2024

(54) SURFACE DISPLAYED ENDOGLYCOSIDASES

(71) Applicant: Clara Foods Co., Daly City, CA (US)

(72) Inventor: Weixi ZHONG, Daly City, CA (US)

(21) Appl. No.: 18/346,095

(22) Filed: Jun. 30, 2023

Related U.S. Application Data
(63) Continuation of application No. PCT/US2021/065703, filed on Dec. 30, 2021.
(60) Provisional application No. 63/132,408, filed on Dec. 30, 2020.

(51) Int. Cl. C12N 9/24; C07K 14/395; C07K 14/435; C12N 1/16

(52) U.S. Cl. CPC C12N 9/2402; C07K 14/395; C07K 14/435; C12N 1/16; C12Y 302/01096; C12R 2001/84

(57) ABSTRACT

The present disclosure provides engineered eukaryotic cells comprising a surface displayed catalytic domain of an endoglycosidase and methods of use.

### CROSS-REFERENCE

This application is a continuation of International Application No. PCT/US2021/065703, filed Dec. 30, 2021, which claims priority to U.S. Application No. 63/132,408, filed Dec. 30, 2020, each of which is hereby incorporated in its entirety by reference herein.

### SEQUENCE LISTING

The instant application contains a Sequence Listing which has been submitted in XML format electronically and is hereby incorporated by reference in its entirety. Said XML copy, created on Sep. 28, 2023, is named 56287US_CRF_sequencelisting.xml and is 435,421 bytes in size.

### BACKGROUND OF THE INVENTION

Recombinant protein expression is a useful method for producing large quantities of animal-free proteins. However, recombinant proteins produced in Pichia pastoris are known to be highly glycosylated. Excessive glycosylation can, at least, raise the risk of immunogenicity in cases where the recombinant protein is intended for consumption and/or therapeutic use. There exists an unmet need for methods and systems for expressing recombinant proteins with reduced amounts of glycosylation.

### SUMMARY

An aspect of the present disclosure is an engineered eukaryotic cell comprising a surface displayed catalytic domain of an endoglycosidase in which the surface displayed catalytic domain of an endoglycosidase is a portion of a fusion protein

In some embodiments, the fusion protein further comprises an anchoring domain of a cell surface protein.

In embodiments, the fusion protein comprises a portion of the endoglycosidase in addition to its catalytic domain.

In various embodiments, the fusion protein comprises substantially the entire amino acid sequence of the endoglycosidase.

In some embodiments, the endoglycosidase is endoglycosidase H.

In embodiments, the fusion protein comprises an amino acid sequence that is at least 95%, 96%, 97%, 98%, 99%, or 100% identical to SEQ ID NO: 1 or SEQ ID NO:2.

In various embodiments, the fusion protein comprises a portion of the cell surface protein in addition to its anchoring domain.

In some embodiments, the fusion protein comprises substantially the entire amino acid sequence of the cell surface protein.

In embodiments, the cell surface protein is selected from Sed1p, Flo5-2, or Flo11.

In various embodiments, the fusion protein comprises an amino acid sequence that is at least 95% identical to one of SEQ ID NO: 3 to SEQ ID NO: 7 and SEQ ID NO: 20.

In some embodiments, the anchoring domain stably attaches the fusion protein to the extracellular surface of the cell.

In embodiments, upon translation, the fusion protein comprises a signal peptide and/or a secretory signal.

In various embodiments, the anchoring domain is N-terminal to the catalytic domain in the fusion protein. In some cases, the fusion protein comprises a linker C-terminal to the anchoring domain.

In some embodiments, the anchoring domain is C-terminal to the catalytic domain in the fusion protein. In some cases, the fusion protein comprises a linker N-terminal to the anchoring domain.

In embodiments, the cell surface protein is Sed1p and the endoglycosidase is endoglycosidase H. In some cases, the fusion protein comprises an amino acid sequence that is at least 95% identical to SEQ ID NO: 9 or SEQ ID NO: 10.

In various embodiments, the cell surface protein is Flo5-2 or Flo11 and the endoglycosidase is endoglycosidase H. In some cases, the fusion protein comprises an amino acid sequence that is at least 95% identical to SEQ ID NO: 11 or SEQ ID NO: 12. In some cases, the fusion protein comprises an amino acid sequence that is at least 95% identical to SEQ ID NO: 13 or SEQ ID NO: 14.

In various embodiments, the engineered eukaryotic cell comprises a mutation in its AOX1 gene and/or its AOX2 gene.

In some embodiments, the engineered eukaryotic cell is a yeast cell. In some cases, the yeast cell is a Pichia species.

In embodiments, the engineered eukaryotic cell further comprises a genomic modification that overexpresses a secretory glycoprotein. In some cases, the secretory glycoprotein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In various embodiments, the cell lacks a genomic modification that overexpresses a secretory glycoprotein.

In some embodiments, the engineered eukaryotic cell further comprises a nucleic acid sequence that encodes the fusion protein. In some cases, the nucleic acid sequence that encodes the fusion protein is integrated into the cell's genome. In some cases, the nucleic acid sequence that encodes the fusion protein is extrachromosomal. In some cases, the nucleic acid sequence comprises an inducible promoter. The inducible promoter may be an AOX1, DAK2, PEX11, FLD1, FGH1, DAS2, CAT1, MDH3, HAC1, BiP, RAD30, RVS161-2, MPP10, THP3, or GBP2 promoter. The nucleic acid sequence may comprise an AOX1, TDH3, RPS25A, or RPL2A terminator. The nucleic acid sequence may encode a signal peptide and/or a secretory signal. The nucleic acid sequence may comprise codons that are optimized for the species of the engineered cell.

Yet another aspect of the present disclosure is an method for deglycosylating a secreted glycoprotein. The method comprising contacting a secreted protein with a fusion protein anchored to engineered eukaryotic cell of any herein disclosed aspect or embodiment, thereby providing a deglycosylated secreted glycoprotein.

In embodiments, the secreted glycoprotein is expressed by the engineered eukaryotic cell.

In various embodiments, the fusion protein anchored to an engineered eukaryotic cell is more effective at deglycosylating the secreted protein than an intracellular endoglycosidase. In some cases, the intracellular endoglycosidase is located within a Golgi vesicle.

In some embodiments, the intracellular endoglycosidase is linked to a membrane associating domain. In some cases, the membrane associating domain comprises an amino acid sequence of OCH1.

In embodiments, the secreted protein is expressed by a cell other than the engineered eukaryotic cell.

In various embodiments, the method further comprises a step of isolating the deglycosylated secreted protein. In some cases, the method further comprises a step of drying the deglycosylated secreted protein.

In some embodiments, the secreted protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In an aspect, the present disclosure provides a method for deglycosylating a plurality of secreted glycoproteins. The method comprising contacting the plurality of secreted glycoproteins with a population of engineered eukaryotic cells of any herein disclosed aspect or embodiment, thereby providing a plurality of deglycosylated secreted glycoproteins.

In embodiments, substantially every secreted glycoprotein in the plurality of secreted proteins is deglycosylated upon contact with the population of engineered eukaryotic cells.

In various embodiments, the amount of deglycosylation of the secreted glycoproteins is not increased by further contacting the secreted protein with an isolated endoglycosidase.

In some embodiments, the amount of deglycosylation of the secreted glycoproteins is more than the amount obtained from a population of cells that express an intracellular endoglycosidase.

In embodiments, the method further comprises a step of isolating the plurality of deglycosylated secreted proteins. In some cases, the method further comprises a step of drying the plurality of deglycosylated secreted proteins.

In various embodiments, the secreted protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In another aspect, the present disclosure provides a method for expressing a fusion protein comprising an anchoring domain of a cell surface protein and a catalytic domain of an endoglycosidase, the method comprising obtaining the engineered eukaryotic cell of any herein disclosed aspect or embodiment and culturing the engineered eukaryotic cell under conditions that promote expression of the fusion protein.

In some embodiments, when the engineered eukaryotic cell comprises a nucleic acid sequence that encodes the fusion protein and comprises an inducible promoter, culturing the engineered eukaryotic cell under conditions that promote expression of the fusion protein comprises contacting the cell with an agent that activates the inducible promoter. In some cases, the inducible promoter is an AOX1, DAK2, PEX11 promoter and the agent that activates the inducible promoter is methanol.

In yet another aspect, the present disclosure provides a population of engineered eukaryotic cells of any herein disclosed aspect or embodiment.

An aspect of the present disclosure is a bioreactor comprising the population of engineered eukaryotic cells of any herein disclosed aspect or embodiment.

Another aspect of the present disclosure is a composition comprising an engineered eukaryotic cell of any herein disclosed aspect or embodiment and a secreted glycoprotein.

In embodiments, the secreted glycoprotein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In an aspect, the present disclosure provides a composition comprising an engineered eukaryotic cell of any herein disclosed aspect or embodiment, a secreted protein that has been deglycosylated, and one or more oligosaccharides cleaved from the secreted protein.

In various embodiments, the secreted glycoprotein is an animal protein, e.g., egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In another aspect, the present disclosure provides a engineered eukaryotic cell which expresses a surface displayed catalytic domain of endoglycosidase H in which the catalytic domain is directly or indirectly tethered to the exterior surface of the cell.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an SDS-PAGE gel demonstrating that a surface displayed EndoH-Sed1p fusion protein is capable of deglycosylating a glycoprotein. Left two lanes show heavy glycosylated species when the secreted glycoprotein is not contacted by a surface displayed fusion protein comprises whereas engineered cells expressing the surface displayed EndoH-Sed1p fusion protein cleaved off the glycoprotein's oligosaccharides, leaving lighter, deglycosylated protein bands.

FIG. 2 shows an SDS-PAGE gel demonstrating that, in bioreactor cultures, engineered cells expressing the EndoH-Sed1p fusion protein cleaved off the glycoprotein's oligosaccharides, leaving faster migrating, deglycosylated protein bands.

### DETAILED DESCRIPTION

### Introduction

The present disclosure provides engineered eukaryotic cells comprising a surface displayed catalytic domain of an endoglycosidase and methods of use.

Surface displaying a catalytic domain of an endoglycosidase provides efficient extracellular deglycosylation of glycoproteins. A glycoprotein is a protein that carries carbohydrates covalently bound to their peptide backbone. It is known that approximately half of all proteins typically expressed in a cell undergo glycosylation, which entails the covalent addition of sugar moieties (e.g., oligosaccharides) to specific amino acids. Most soluble and membrane-bound proteins expressed in the endoplasmic reticulum are glycosylated to some extent, including secreted proteins, surface receptors and ligands, and organelle-resident proteins. Additionally, some proteins that are trafficked from the Golgi to the cell wall and/or to the extracellular environment are also glycosylated. Lipids and proteoglycans can also be glycosylated, significantly increasing the number of substrates for this type of modification. In particular, many cell wall proteins are glycosylated.

Protein glycosylation has multiple functions in a cell. In the ER, glycosylation is used to monitor the status of protein folding, acting as a quality control mechanism to ensure that only properly folded proteins are trafficked to the Golgi. Oligosaccharides on soluble proteins can be bound by specific receptors in the trans Golgi network to facilitate their delivery to the correct destination. These oligosaccharides can also act as ligands for receptors on the cell surface to mediate cell attachment or stimulate signal transduction pathways. Because they can be very large and bulky, oligosaccharides can affect protein—protein interactions by either facilitating or preventing proteins from binding to cognate interaction domains.

In general, a glycoprotein's oligosaccharides are important to the protein's function. Consequently, should a glycoprotein be deglycosylated intracellularly, once the protein has reached its final destination (if ever), and in a deglycosylated state, the protein may have a lessened and/or an absent activity.

When it is desirable to deglycosylate a recombinant glycoprotein for inclusion in composition for human or animal use (e.g., a food product, drink product, nutraceutical, pharmaceutical, or cosmetic), the recombinant glycoprotein may be contacted with an isolated endoglycosidase that is capable of cleave sugar chains from the glycoprotein. For this, the isolated endoglycosidase may be added to a culturing vessel such that the recombinant glycoprotein is deglycosylated once secreted into its culturing medium. Alternately, a recombinant glycoprotein that has been separated from its culturing medium may be subsequently incubated with the isolated endoglycosidase. Although both of these methods may have effectiveness in providing deglycosylated recombinant proteins, they both increase, at least, the time, expense, and inefficiency involved with manufacturing deglycosylated recombinant proteins. When preparing deglycosylated recombinant proteins for human or animal use, e.g., in a consumable composition, it is preferable, and in some cases, necessary due to regulatory requirements, for the final recombinant protein be free of contaminants. One such contaminant is the endoglycosidase itself. In this case, the endoglycosidase must be removed in part or completely from the final recombinant protein product. This removal would entail multiple purification steps that both increase the expense due to these additional steps and reduce the amount of recombinant protein produced, as some protein would be lost during the various purifications. Also, these purification steps would extend the time for manufacturing the recombinant protein product, thereby reducing efficiency of the process. Moreover, when a recombinant glycoprotein is combined with the endoglycosidase, either in a culturing medium or after the recombinant glycoprotein has been separated from its medium, there is no guarantee that each recombinant glycoprotein will come into contact with an endoglycosidase; to ensure sufficient deglycosylation, the glycoprotein and endoglycosidase must remain in a solution for an extended period of time. This extension of time further reduces the efficiency of the manufacturing process. Finally, purchasing the isolated endoglycosidase or manufacturing the isolated endoglycosidase in house would incur additional expenses. Together, there is an unmet need for manufacturing deglycosylated recombinant protein that is effective and efficient. The methods and systems of the present disclosure satisfy this unmet need.

In the present disclosure, an endoglycosidase is localized to the extracellular surface of a cell, i.e., is surface displayed. This way, the endoglycosidase is unlikely to contact an intracellular, membrane-associated, or cell wall glycoprotein, thereby lowering the opportunity for the endoglycosidase to remove a needed oligosaccharide from the glycoprotein. Instead, the surface displayed endoglycosidase primarily deglycosylates proteins found in the extracellular space, e.g., secreted recombinant proteins. Accordingly, the present disclosure provides recombinant cells having the means to deglycosylate secreted glycoproteins proteins and having a reduced likelihood of undesirably deglycosylating its own intracellular, membrane bound, or cell wall glycoproteins.

Additionally, since the surface displayed endoglycosidase is securely attached to the recombinant cell, it is not released into and present in a culturing medium. Thus, there is no need to separate the endoglycosidase from the secreted recombinant protein when making a generally contaminant-free recombinant protein product. In other words, the use of surface displayed endoglycosidase avoids the added expense, time, and inefficiency, as described above, that is needed to later remove the endoglycosidase when manufacturing a recombinant protein product for human or animal use, e.g., in a consumable composition.

### Fusion Proteins

Aspects of the present disclosure provide an engineered eukaryotic cell comprising a surface displayed catalytic domain of an endoglycosidase. The surface displayed catalytic domain of the endoglycosidase is included in a fusion protein expressed by the cell. As used herein, the term “catalytic domain” comprises a portion of an endoglycosidase that provides catalytic activity.

A fusion protein is a protein consisting of at least two domains that are normally encoded by separate genes but have been joined so that they are transcribed and translated as a single unit; thereby, producing a single (fused) polypeptide.

In the present disclosure, a fusion protein comprises at least a catalytic domain of an endoglycosidase and an anchoring domain of a cell surface protein.

A fusion protein may further comprise linkers that separate the two domains. Linkers can be flexible or rigid; they can be semi-flexible or semi-rigid. Separating the two domains, may promote activity of the catalytic domain in that it reduces steric hindrance upon the catalytic site which may be present if the catalytic site is too closely positioned relative to an anchoring domain. Additionally, a linker may further project the catalytic domain into the extracellular space, thereby increasing the likelihood that the catalytic domain will encounter and cleave glycoproteins.

When a linker is present, a fusion protein may have a general structure of: N terminus -(a)-(b)-(c)-C terminus, wherein (a) is comprises a first domain, (b) is one or more linkers, and (c) is a second domain. The first domain may comprise a catalytic domain of an enzyme and the second domain may comprise an anchoring domain of a cell surface protein. Alternately, the first domain may comprise an anchoring domain of a cell surface protein and the second domain may comprise a catalytic domain of an enzyme. In some embodiments, the anchoring domain is N-terminal to the catalytic domain in the fusion protein. The fusion protein may comprise a linker C-terminal to the anchoring domain. In other embodiments, the anchoring domain is C-terminal to the catalytic domain in the fusion protein. The fusion protein may comprise a linker N-terminal to the anchoring domain.

In some embodiments, a fusion protein comprises more than one anchoring domains of a cell surface protein. In such embodiments, the fusion protein may have a general structure of: N terminus -(a)-(b)-(c)-(d)-(e)- C terminus, wherein (a) and (e) comprise anchoring domains of a cell surface protein, (b) and (d) are linkers (which may be the same linker or different) and (c) is comprises a catalytic domain of an enzyme.

Linkers useful in fusion proteins may comprise one or more sequences of SEQ ID NO: 21 to SEQ ID NO: 25. In one example, a tandem repeat (of two, three, four, five, six, or more copies) of a linker, e.g., of SEQ ID NO: 22 or SEQ ID NO: 23, is included in a fusion protein.

In embodiments, a fusion protein comprises a Glu-Ala-Glu-Ala (EAEA; SEQ ID NO: 21) spacer dipeptide repeat. The EAEA is a removable signal that promotes yields of an expressed protein in certain cell types.

Other linkers are well-known in the art and can be substituted for the linkers of SEQ ID NO: 21 to SEQ ID NO: 25. For example, In embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some cases, the linker is about 59 amino acids long.

The length of a linker may be important to the effectiveness of a surface displayed endoglycosidase catalytic domain. For example, if a linker is too short, then the catalytic domain of the endoglycosidase may not project far enough away from the cell surface such that it is incapable of interacting with a glycoprotein. In this case, the catalytic domain may be buried in the cell wall and/or among other cell surface proteins or sugars. On the other hand, the linker may be too long and/or too rigid to allow adequate contact between a secreted glycoprotein and the catalytic domain of the endoglycosidase.

The secondary structure of a linker may also be important to the effectiveness of a surface displayed endoglycosidase catalytic domain. More specifically, a linker designed to have a plurality of distinct regions may provide additional flexibility to the fusion protein. As examples, a linker having one or more alpha helices may be superior to a linker having no alpha helices.

The longer linker of (SEQ ID NO: 25) comprises three subsections: an N-terminal flexible GS linker with higher S content (SEQ ID NO: 295), a rigid linker that forms four turns of an alpha helix (SEQ ID NO: 24), and a flexible GS linker with much higher G content (SEQ ID NO: 296) on its C-terminus. Linkers containing only G's and S's in repetitive sequences are commonly used in fusion proteins as flexible spacers that do not introduce secondary structure. In some cases, the ratio of G to S determines the flexibility of the linker. Linkers with higher G content may be more flexible than linkers with higher S content. The structure of the linker of SEQ ID NO: 25 is designed to mimic multi-domain proteins in nature, which often uses alpha helices (sometimes multiple) to separate as well as orient their domains spatially. In fusion proteins of the present disclosure, a complex linker, such as that of SEQ ID NO: 25 can be viewed as a multi-domain protein with the catalytic domain of an endoglycosidase and an anchoring domain of a cell surface protein being separate functional domains.

In various embodiments, the fusion protein comprises a linker having an amino acid sequence that is at least 95%, 96%, 97%, 98%, 99%, or 100% identical to SEQ ID NO: 25.

In embodiments, the linker is substantially comprised of glycine and serine residues (e.g. about 30%, or about 40%, or about 50%, or about 60%, or about 70%, or about 80%, or about 90%, or about 95%, or about 96%, or about 97%, or about 98%, or about 99%, or about 100% glycines and serines).

### Endoglycosidases

An Endoglycosidase is an enzyme that releases oligosaccharides from glycoproteins or glycolipids. Unlike exoglycosidases, endoglycoidases cleave polysaccharide chains between residues that are not the terminal residue and break the glycosidic bonds between two sugar monomer in the polymer. When an endoglycosidase cleaves, it releases an oligosaccharide product.

Numerous endoglycosidases have been characterized, cloned, and/or purified. These include Endoglycosidase D, Endoglycosidase F1, Endoglycosidase F2, Endoglycosidase F3, Endoglycosidase H, Endoglycosidase Hf, Endoglycosidase S, Endoglycosidase T, Endoglycoceramidase I, O-Glycosidase, Peptide-N-Glycosidase A (PNGaseA), and PNGaseF.

Normally, an endoglycosidase comprises at least a catalytic domain which is responsible for cleaving an oligonucleotide from a glycoprotein. The endoglycosidase may also comprise domains that help recognize an oligosaccharide and/or the glycoprotein itself. The endoglycosidase may further comprise domains that help facilitate, e.g., positioning of the oligosaccharide and/or glycoprotein itself, cleavage of the oligosaccharide.

In various embodiments, a fusion protein comprises at least the catalytic domain of the endoglycosidase. In some cases, a fusion protein comprises a portion of the endoglycosidase in addition to its catalytic domain. In some embodiments, a fusion protein comprises substantially the entire amino acid sequence of the endoglycosidase.

### Endoglycosidase H

In some cases, the endoglycosidase is endoglycosidase H.

Endoglycosidase H (Endo H); Endo-beta-N-acetylglucosaminidase H (EC:3.2.1.96); DI-N-acetylchitobiosyl beta-N-acetylglucosaminidase H; Mannosyl-glycoprotein endo-beta-N-acetyl-glucosaminidase H is a highly specific endoglycosidase which cleaves asparagine-linked mannose rich oligosaccharides, but not highly processed complex oligosaccharides from glycoproteins. EndoH hydrolyzes (cleaves) the bond in the diacetylchitobiose core of the oligosaccharide between two N-acetylglucosamine (GlcNAc) subunits directly proximal to the asparagine residue, generating a truncated sugar molecule that is released intact and one N-acetylglucosamine residue remaining on the asparagine.

Variants of the known amino acid sequence of endoH may be determined by consulting the literature. e.g. Robbins et al., “Primary structure of the Streptomyces enzyme endo-beta-N-acetylglucosaminidase H.” J. Biol. Chem. 259:7577-7583 (1984); Rao et al., “Crystal structure of endo-beta-N-acetylglucosaminidase H at 1.9-A resolution: active-site geometry and substrate recognition.” Structure 3:449-457 (1995); Rao et al., “Mutations of endo-beta-N-acetylglucosaminidase H active site residue Asp130 and Glu132: activities and conformations.” Protein Sci. 8:2338-2346 (1999); the contents of which are incorporated by reference in their entirety. For example, Rao et al., (1999) teaches specific mutations that reduce (e.g., from 1.25% to 0.05% of wild-type activity) or completely obliterate enzymatic activity. Thus, a variant of endoH which comprises a substitution at Asp172 and/or Glu174 (with respect to SEQ ID NO: 2) would be understood to have undesired activity. Based on the published structural and functional analyses and routine experimentation, it could be readily determined those amino acids within endoH that could be substituted and would retain enzymatic activity and which amino acids could not be substituted.

In embodiments, the endoH that is surface displayed, e.g., is part of a fusion protein, comprises an amino acid sequence of SEQ ID NO: 1 or SEQ ID NO: 2. The amino acid sequence of SEQ ID NO: 1 lacks an N-terminal signal peptide that is present in SEQ ID NO: 2. The endoH may be a variant of SEQ ID NO: 1 or SEQ ID NO: 2. The variant may have at least or about 70%, 75%, 80%, 85%, 90%, 92%, 95%, 96%, 97%, 98%, 99%, or 100% sequence identity with one of SEQ ID NO: 1 or SEQ ID NO: 2.

### Surface Display

Aspects of the present disclosure include engineered eukaryotic cells comprising a surface displayed catalytic domain of an endoglycosidase.

In embodiment, surface display occurs by attachment of the catalytic domain to the extracellular surface of the cell via an anchoring domain of a cell surface protein. In the present disclosure, the catalytic domain and anchoring domain are present in a fusion protein, optionally, separated by one or more linkers.

Surface display is understood as the projection of a protein, e.g., a fusion protein, out from a cell's surface and/or from the cell's membrane and into the extracellular space, e.g., into the growth medium in which the engineered eukaryotic cell is being cultured. By projecting into the extracellular space, a surface displayed fusion protein is positioned to interact with soluble glycoproteins present in the extracellular space. Alternately, a surface displayed fusion protein is positioned to interact with cell-associated proteins on adjacent cells. When the surface displayed fusion protein comprise a catalytic domain of an enzyme, e.g., an endoglycosidase, and especially, endoH, the catalytic domain is positioned to cleave off oligonucleotides from soluble glycoproteins present in the extracellular space or cleave off oligonucleotides from cell-associated glycoproteins on adjacent cells.

In some cases, the cell that expresses a surface displayed fusion protein also expresses (co-expresses) a secreted glycoprotein. This co-expression simplifies the production of deglycosylated proteins in that only one engineered cell needs to be produced and cultured. Moreover, as the secreted glycoprotein is released by the engineered cell, it is an enhanced likelihood of contacting the fusion protein that is located on the surface of the same cell.

In alternate case, the cell that expresses the fusion protein is different from the cell that secretes the glycoprotein. An advantage of this configuration is that an engineered cell that optimally expresses a fusion protein can be co-cultured with an engineered cell that optimally expresses a secreted glycoprotein.

To ensure that a fusion protein is surface displayed and remains attached to the extracellular surface of a cell rather than being secreted and released into the extracellular space, a fusion protein comprises an anchoring domain from a cell surface protein. These anchoring domains either bind to a component of the cell's membrane or its cell wall or the anchoring domain comprises a motif that is used to attach the protein to the cell's membrane, e.g., via a glycosylphosphatidylinositol (GPI) anchor. Thus, the anchoring domain stably attaches the fusion protein to the extracellular surface of the engineered cell.

In some cases, a fusion protein comprises a portion of the cell surface protein in addition to its anchoring domain. In embodiments, a fusion protein comprises substantially the entire amino acid sequence of the cell surface protein.

In various embodiments, the cell surface protein is selected from Sed1p, Flo5-2, Flo11, Saccharomyces cerevisiae Flo5, CWP, and PIR.

Sed1p is a major component of the Saccharomyces cerevisiae cell wall. It is required to stabilize the cell wall and for stress resistance in stationary-phase cells. See, e.g., the world wide web (at) uniprot.org/uniprot/Q01589. It is believed that Asn 318 (with respect to SEQ ID NO: 3) is the most likely candidate for the GPI attachment site in Sed1p. In some embodiments, a fusion protein comprising a Sed1p anchoring domain has a sequence having at least 95% or more sequence identity with SEQ ID NO: 3 or SEQ ID NO: 4. In some cases, the sequence identity may be greater than or about 90%, 95%, 96%, 97%, 98%, 99%, or 100%. In various embodiments, the Sed1p anchoring domain of a fusion protein of the present disclosure comprises a GPI attachment site; thus, the anchoring domain may only require a short fragment of SEQ ID NO: 3 or SEQ ID NO: 4, i.e., a fragment that is 5, 10, 25, 50, 100, 200, or 300 or more amino acids in length, as long as it is capable of projecting the catalytic domain of the fusion protein into the extracellular space. In some embodiments, the anchoring domain comprises, at least, Sed1p's GPI attachment site.

In some cases, the cell surface protein is Sed1p and the endoglycosidase is endoglycosidase H. The fusion protein may comprise an amino acid sequence that is at least 95% identical to SEQ ID NO: 9 or SEQ ID NO: 10. In some cases, the sequence identity may be greater than or about 90%, 95%, 96%, 97%, 98%, 99%, or 100% to SEQ ID NO: 9 or SEQ ID NO: 10.

Komagataella phaffii Flo5-2 is considered to be an ortholog of both Saccharomyces Flo1 and Flo5. See, e.g., the world wide web (at) uniprot.org/uniprot/F2QXPO. The two Saccharomyces flocculation proteins are highly similar in their amino acid sequence, only significantly differing in the length of the linker portion used to extend the protein past the cell wall. The Saccharomyces flocculation proteins are cell wall proteins that participate directly in adhesive cell-cell interactions during yeast flocculation, a reversible, asexual process in which cells adhere to form aggregates (flocs) consisting of thousands of cells. The lectin-like proteins stick out of the cell wall of flocculent cells and selectively bind mannose residues in the cell walls of adjacent cells. Literature on Saccharomyces Flo1p shows that monomeric mannose added to the media can prevent flocculation, suggesting that flocculation by Flo1p results from binding to mannose in the cell wall and free-floating mannose can compete for the binding spot. Thus, the flocculation family of proteins are useful in the present disclosure, for, at least, two reasons. First, they generally extend relative far from the cell wall, and, second, it is believed that they bind and capture some exopolysaccharides. Notably, Flo5-2 has a GPI anchor site towards its C-terminus which can tether the protein to a cell's membrane. Therefore, a fusion protein comprising an anchoring domain of Flo5-2 may anchor the fusion protein to the extracellular surface of an engineered cell via its GPI anchor or by the domain's interaction with exopolysaccharides located on the extracellular surface of an engineered cell. Moreover, without wishing to be bound by theory, inclusion of an anchoring domain of Flo5-2 may promote capture of a secreted glycoprotein for deglycosylation.

In some embodiments, a fusion protein comprising a Flo5-2 anchoring domain has a sequence that has 95% or more sequence identity with SEQ ID NO: 5 or SEQ ID NO: 6. In some cases, the sequence identity may be greater than or about 90%, 95%, 96%, 97%, 98%, 99%, or 100%. In various embodiments, the Flo5-2 anchoring domain of a fusion protein of the present disclosure comprises a GPI attachment site; thus, the anchoring domain may only require a short fragment of SEQ ID NO: 5 or SEQ ID NO: 6, i.e., a fragment that is 5, 10, 25, 50, 100, 200, 300, 400, 500, 600, 700, 800, 900, or 1000 or more amino acids in length, as long as it is capable of projecting the catalytic domain of the fusion protein into the extracellular space. In some embodiments, the anchoring domain comprises, at least, Flo5-2's GPI attachment site. In some embodiments, the anchoring domain lacks Flo5-2's GPI attachment site yet retains the ability to capture exopolysaccharides and retain the fusion protein at the extracellular surface.

In some cases, the cell surface protein is Flo5-2 and the endoglycosidase is endoglycosidase H. The fusion protein may comprise an amino acid sequence that is at least 95% identical to SEQ ID NO: 11 or SEQ ID NO: 12. In some cases, the sequence identity may be greater than or about 90%, 95%, 96%, 97%, 98%, 99%, or 100% to SEQ ID NO: 11 or SEQ ID NO: 12.

Saccharomyces cerevisiae Flo5 has a GPI anchor site towards its C-terminus which can tether the protein to a cell's membrane. Therefore, a fusion protein comprising an anchoring domain of Flo5 may anchor the fusion protein to the extracellular surface of an engineered cell via its GPI anchor or by the domain's interaction with exopolysaccharides located on the extracellular surface of an engineered cell. Moreover, without wishing to be bound by theory, inclusion of an anchoring domain of Flo5 may promote capture of a secreted glycoprotein for deglycosylation.

In some embodiments, a fusion protein comprising a Saccharomyces cerevisiae Flo5 anchoring domain has a sequence that has 95% or more sequence identity with SEQ ID NO: 20. In some cases, the sequence identity may be greater than or about 90%, 95%, 96%, 97%, 98%, 99%, or 100%. In various embodiments, the Flo5 anchoring domain of a fusion protein of the present disclosure comprises a GPI attachment site; thus, the anchoring domain may only require a short fragment of SEQ ID NO: 20, i.e., a fragment that is 5, 10, 25, 50, 100, 200, 300, 400, 500, 600, 700, 800, 900, or 1000 or more amino acids in length, as long as it is capable of projecting the catalytic domain of the fusion protein into the extracellular space. In some embodiments, the anchoring domain comprises, at least, Flo5's GPI attachment site. In some embodiments, the anchoring domain lacks Flo5's GPI attachment site yet retains the ability to capture exopolysaccharides and retain the fusion protein at the extracellular surface.

In some cases, the cell surface protein is Saccharomyces cerevisiae Flo5 and the endoglycosidase is endoglycosidase H. The fusion protein may comprise an amino acid sequence that is at least 95% identical to SEQ ID NO: 293. In some cases, the sequence identity may be greater than or about 90%, 95%, 96%, 97%, 98%, 99%, or 100% to SEQ ID NO: 293.

Flo11 is another GPI-anchored cell surface glycoprotein (flocculin). See, e.g., the world wide web (at) uniprot.org/uniprot/F2QRD4. Flo11 is believed to be required for pseudohyphal and invasive growth, flocculation, and biofilm formation. It is a major determinant of colony morphology and required for formation of fibrous interconnections between cells. Like the other yeast flocculation proteins, its adhesive activity is inhibited by mannose, but not by glucose, maltose, sucrose or galactose. Thus, use of Flo11 in a fusion protein of the present disclosure may be useful extending the fusion protein relatively far from the cell wall, and for binding and capturing some exopolysaccharides. Like, Flo5-2, Flo11 has a GPI anchor site towards its C-terminus which can tether the protein to a cell's membrane. Therefore, a fusion protein comprising an anchoring domain of Flo11 may anchor the fusion protein to the extracellular surface of an engineered cell via its GPI anchor or by the domain's interaction with exopolysaccharides located on the extracellular surface of an engineered cell. Moreover, without wishing to be bound by theory, inclusion of an anchoring domain of Flo11 may promote capture of a secreted glycoprotein for deglycosylation.

In some embodiments, a fusion protein comprising a Flo11 anchoring domain has a sequence that has 95% or more sequence identity with SEQ ID NO: 7 or SEQ ID NO: 8. In some cases, the sequence identity may be greater than or about 90%, 95%, 96%, 97%, 98%, 99%, or 100%. In various embodiments, the Flo11 anchoring domain of a fusion protein of the present disclosure comprises a GPI attachment site; thus, the anchoring domain may only require a short fragment of SEQ ID NO: 7 or SEQ ID NO: 8, i.e., a fragment that is 5, 10, 25, 50, 100, 200, 300, 400, 500, 600, 700, 800, 900, or 1000 or more amino acids in length, as long as it is capable of projecting the catalytic domain of the fusion protein into the extracellular space. In some embodiments, the anchoring domain comprises, at least, Flo11's GPI attachment site. In some embodiments, the anchoring domain lacks Flo11's GPI attachment site yet retains the ability to capture exopolysaccharides and retain the fusion protein at the extracellular surface.

In some cases, the cell surface protein is Flo11 and the endoglycosidase is endoglycosidase H. The fusion protein may comprise an amino acid sequence that is at least 95% identical to SEQ ID NO: 13 or SEQ ID NO: 14. In some cases, the sequence identity may be greater than or about 90%, 95%, 96%, 97%, 98%, 99%, or 100% to SEQ ID NO: 13 or SEQ ID NO: 14.

### Engineered Eukaryotic Cells

The present disclosure relates to engineered eukaryotic cells. These engineered cells are transfected to express a surface displayed catalytic domain of an endoglycosidase. In various embodiments, the engineered cells are transfected to express a surface displayed fusion protein comprising a catalytic domain of an endoglycosidase and an anchoring domain of a cell surface protein.

In some cases, the engineered eukaryotic cell is a yeast cell, e.g., yeast cell that is a Pichia species

A fusion protein may be expressed by the cell by nucleic acid sequence, e.g., an expression cassette, that is stably integrated into a cell's chromosome. Alternately, a fusion protein may be expressed by the cell by an extrachromosomal nucleic acid sequence, e.g., plasmid, vector, or YAC which comprises an expression cassette. Any method for transfecting cells with suitable constructs that express the fusion protein may be used.

An expression cassette is any nucleic acid sequence that contains a subsequence that codes for a transgene and can confer expression of that subsequence when contained in a microorganism and is heterologous to that microorganism. It may comprise one or more of a coding sequence, a promoter, and a terminator. It may encode a secretory signal. It may further encode a signal sequence. In some embodiments, a nucleic acid sequence, e.g., which is expressed by a recombinant cell, may comprise an expression cassette.

The expression cassettes useful herein can be obtained using chemical synthesis, molecular cloning or recombinant methods, DNA or gene assembly methods, artificial gene synthesis, PCR, or any combination thereof. Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide comprising a desired sequence can be inserted into a suitable cloning or expression vector, and the cloning or expression vector in turn can be introduced into a suitable host cell for replication and amplification. Suitable cloning vectors may be constructed according to standard techniques, or may be selected from a large number of cloning vectors available in the art. While the cloning vector selected fvmay vary according to the host cell intended to be used, useful cloning vectors will generally have the ability to self-replicate, may possess a single target for a particular restriction endonuclease, and/or may carry genes for a marker that can be used in selecting clones containing the expression vector. Methods for obtaining cloning and expression vectors are well-known (see, e.g., Green and Sambrook, Molecular Cloning: A Laboratory Manual, 4th edition, Cold Spring Harbor Laboratory Press, New York (2012)), the contents of which is incorporated herein by reference in its entirety.

In some cases, it is desirable for a engineered cell to express multiple copies of the fusion protein and/or to control expression of the fusion protein. Thus, a nucleic acid sequence or expression cassette may comprise a constitutive promoter, inducible promoter, and hybrid promoter. A promoter refers to a polynucleotide subsequence of nucleic acid sequence or an expression cassette that is located upstream, or 5′, to a coding sequence and is involved in initiating transcription of the coding sequence when the nucleic acid sequence or expression cassette is integrated into a chromosome or located extrachromosomally in a host cell.

Notably, in some cases, it is undesirable for a cell to excessively express the fusion protein. The main purpose of the recombinant cells of the present disclosure is to produce the recombinant glycoproteins, e.g., for inclusion in composition for human or animal use. Should a cell express excessive amounts of the fusion protein, then the transcriptional and translational machinery dedicated to producing the fusion protein cannot be used to produce the recombinant glycoproteins. If so, the cell may become stressed and produce either less recombinant glycoproteins and/or may produce undesirable byproducts. Thus, in some embodiments, a nucleic acid encoding a fusion protein is fused to a weak promoter or to an intermediate strength promoter rather than a strong promoter.

In embodiments, the nucleic acid sequence or expression cassette comprises an inducible promoter. The inducible promoter may be an AOX1, DAK2, PEX11, FLD1, FGH1, DAS2, CAT1, MDH3, HAC1, BiP, RAD30, RVS161-2, MPP10, THP3, or GBP2 promoter. In some embodiments, the promoter used may have a sequence that has 95% or more sequence identity with any of SEQ ID NO: 26 to SEQ ID NO: 40. In some cases, the sequence identity may be greater than or about 90%, 92%, 95%, 96%, 97%, 98%, 99%, or 100% sequence identity with any of SEQ ID NO: 26 to SEQ ID NO: 40.

Useful promoters may be selected from acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, me10, MET3, methanol oxidase (MOX), nmt1, NSP, pcbC, PETS, phosphoglycerate kinase (PGK, PGK1), pho 1, PH05, PH089, phosphatidylinositol synthase (PIS1), PYK1, pyruvate kinase (pki1), RPS7, sorbitol dehydrogenase (SDH), 3-phosphoserine aminotransferase (SERI), SSA4, SV40, TEF, translation elongation factor 1 alpha-(TEF1), THI11, homoserine kinase (THR1), tpi, TPS1, triose phosphate isomerase (TPI1), XRP2, YPT1, GCW14, GAP, a sequence or subsequence chosen from SEQ ID NO: 26 to SEQ ID NO: 48, and any combination thereof. In some cases, the sequence identity may be greater than or about 90%, 92%, 95%, 96%, 97%, 98%, 99%, or 100% sequence identity with any of SEQ ID NO: 26 to SEQ ID NO: 48.

In embodiments, the nucleic acid sequence or expression cassette comprises a terminator sequence. A terminator is a section of nucleic acid sequence that marks the end of a gene during transcription. In some cases, the terminator is an AOX1, TDH3, RPS25A, or RPL2A terminator. In some embodiments, the terminator used may have a sequence that has 95% or more sequence identity with any of SEQ ID NO: 53 to SEQ ID NO: 56. In some cases, the sequence identity may be greater than or about 90%, 92%, 95%, 96%, 97%, 98%, 99%, or 100% sequence identity with any of SEQ ID NO: 53 to SEQ ID NO: 56.

Certain combinations of promoter and terminator may provide more preferred expression of the fusion protein and/or more preferred activity of the fusion protein, e.g., in deglycosylating glycoproteins. It is well-within the skill of an artisan to determine which combinations of promoters and terminators achieve desirability and which combinations do not.

Moreover, in some cases, the same combination of promoter and terminator may have preferred activity in one strain and have less preferred activity in another strain. Without wishing to be bound by theory, the strain difference may be due to a construct's integration into the host cell's genome or it may be due to epigenetic reasons. It is well-within the skill of an artisan to determine which strains for a certain combination of promoter and terminator achieve desirability and which strains do not.

Additionally, some combinations of promoters and terminators and certain strains perform better when cells are cultured at higher density (e.g., in bioreactors) versus low density cell cultures, as in a high throughput screen. Thus, a combination or strain may appear to be less desirable when assayed in small scale cultures, but may actually be a preferred combination or strain when cultured at higher cell density, which would be the case for commercial scale production of deglycosylated proteins. It is well-within the skill of an artisan to determine the culturing conditions that ensure certain combination of promoter and terminator and specific strains provided desirable amounts of glycoprotein deglycosylation.

In some cases, the nucleic acid sequence or expression cassette encodes a signal peptide and/or a secretory signal. A signal peptide, also known as a signal sequence, targeting signal, localization signal, localization sequence, transit peptide, leader sequence, or leader peptide, may support secretion of a protein or polynucleotide. Extracellular secretion (for the purposes of surface display) of a recombinant or heterologously expressed fusion protein is facilitated by having a signal peptide included in the fusion protein. A signal peptide may be derived from a precursor (e.g., prepropeptide, preprotein) of a protein. Signal peptides may be derived from a precursor of a protein including, but not limited to, acid phosphatase (e.g., Pichia pastoris PHO1), albumin (e.g., chicken), alkaline extracellular protease (e.g., Yarrowia lipolytica XRP2), α-mating factor (α-MF, MATα) (e.g., Saccharomyces cerevisiae), amylase (e.g., α-amylase, Rhizopus oryzae, Schizosaccharomyces pombe putative amylase SPCC63.02c (Amy 1)), β-casein (e.g., bovine), carbohydrate binding module family 21 (CBM21)-starch binding domain, carboxypeptidase Y (e.g., Schizosaccharomyces pombe Cpy 1), cellobiohydrolase I (e.g., Trichoderma reesei CBH1), dipeptidyl protease (e.g., Schizosaccharomyces pombe putative dipeptidyl protease SPBC1711.12 (Dpp1)), glucoamylase (e.g., Aspergillus awamori), heat shock protein (e.g., bacterial Hsp70), hydrophobin (e.g., Trichoderma reesei HBFI, Trichoderma reesei HBFII), inulase, invertase (e.g., Saccharomyces cerevisiae SUC2), killer protein or killer toxin (e.g., 128 kDa pGKL killer protein, α-subunit of the K1 killer toxin (e.g., Kluyveromyces lactis), K1 toxin KILM1, K28 pre-pro-toxin, Pichia acaciae), leucine-rich artificial signal peptide CLY-L8, lysozyme (e.g., chicken CLY), phytohemagglutinin (PHA-E) (e.g., Phaseolus vulgaris), maltose binding protein (MBP) (e.g., Escherichia coli), P-factor (e.g., Schizosaccharomyces pombe P3), Pichia pastoris Dse, Pichia pastoris Exg, Pichia pastoris Pir1, Pichia pastoris Scw, and cell wall protein Pir4 (protein with internal repeats). In some embodiments, the signal peptide used may have a sequence that has 80% or more sequence identity with any of SEQ ID NO: 57 to SEQ ID NO: 156. In some cases, the sequence identity may be greater than or about 90%, 92%, 95%, 96%, 97%, 98%, 99%, or 100% sequence identity with any of SEQ ID NO: 57 to SEQ ID NO: 156. In some cases, the signal peptide used may have a sequence that has 80% or more sequence identity with any of SEQ ID NO: 57 to SEQ ID NO: 61. In some cases, the sequence identity may be greater than or about 90%, 92%, 95%, 96%, 97%, 98%, 99%, or 100% sequence identity with any of SEQ ID NO: 57 to SEQ ID NO: 61.

In various embodiments, a fusion protein comprises an α-mating factor (α-MF, MATα) (e.g., Saccharomyces cerevisiae) secretion signal. In some cases the alpha mating factor signal peptide and secretion signal has a sequence that has 95% or more sequence identity with SEQ ID NO: 290 or SEQ ID NO: 291. In some cases, the sequence identity may be greater than or about 90%, 92%, 95%, 96%, 97%, 98%, 99%, or 100% sequence identity with any of with SEQ ID NO: 290 or SEQ ID NO: 291. The α-mating factor secretion signal targets a fusion protein through the secretory pathway and is removed before exiting the cell.

In some cases, a nucleic acid sequence or expression cassette encodes a selectable marker. The selectable maker may be an antibiotic resistance gene (e.g., zeocin, ampicillin, blasticidin, kanamycin, nourseothricin, chloroamphenicol, tetracycline, triclosan, ganciclovir, and any combination thereof), an auxotrophic marker (e.g., f ade1, arg4, his4, ura3, met2, and any combination thereof).

In various embodiments, a nucleic acid sequence or expression cassette comprises codons that are optimized for the species of the engineered cell, e.g., a yeast cell including a Pichia cell. As known in the art, codon optimization may improve stability and/or increase expression of a recombinant protein, e.g., a fusion protein of the present disclosure. Surprisingly, codon optimization of a nucleic acid sequence or expression cassette my improve the transfection efficiency of the nucleic acid sequence or expression cassette into the genome of a host cell. Codon utilization tables for various species of host cell are publicly available. See, e.g., the world wide web (at) kazusa.or.jp/codon/cgi-bin/showcodon.cgi?species=4922&aa=15&style=N.

Host cells useful for expression fusion proteins of the present disclosure include but are not limited to: Arxula spp., Arxula adeninivorans, Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, Trichoderma vireus, Aspergillus oryzae, Bacillus subtilis, Escherichia coli, Myceliophthora thermophila, Neurospora crassa, Pichia pastoris, Komagataella phaffii and Komagataella pastoris.

Transfection of a host cell with an expression cassette can exploit the natural ability of a host cell to integrate exogenous DNA into its chromosome. This natural ability is well documented for yeast cells, including Pichia cells. In some embodiments an additional vector and or additional elements may be designed to aide (as deemed necessary by one skilled in the art) for the particular method of transfection (e.g. CAS9 and gRNA vectors for a CRISPR/CAS9 based method).

In some cases, a host eukaryotic cell that expresses a fusion protein comprises a mutation in its AOX1 gene and/or its AOX2 gene. A deletion in either the AOX1 gene or AOX2 gene generates a methanol-utilization slow (mutS) phenotype that reduces the strain's ability to consume methanol as an energy source. A deletion in both the AOX1 gene and the AOX2 gene generates a methanol-utilization minus (mutM) phenotype that substantially limits the strain's ability to consume methanol as an energy source. Using an AOX1 mutant and/or AOX2 mutant cell is especially useful in the context of a fusion protein encoded by an expression cassette that comprises a methanol-inducible promoter, e.g., OAX1, DAS1, and FDH1. In this configuration, the host cell does not use methanol as an energy source, thus, when the cell is provided methanol, the methanol is primarily used to activate the methanol-inducible promoter, thereby especially activating the promoter and causing increased expression of the fusion protein.

Another aspect of the present disclosure is a population of engineered eukaryotic cells of any of the herein disclosed aspects or embodiments. The present disclosure further relates to a bioreactor comprising this population of engineered eukaryotic cells.

Yet another aspect of the present disclosure is a method for expressing a fusion protein comprising an anchoring domain of a cell surface protein and a catalytic domain of an endoglycosidase. The method comprises obtaining any herein disclosed engineered eukaryotic cell and culturing the engineered eukaryotic cell under conditions that promote expression of the fusion protein.

The conditions that promote expression of the fusion protein may be standard growth conditions. However, when the engineered eukaryotic cell comprises a nucleic acid sequence that encodes the fusion protein and comprises an inducible promoter, culturing the engineered eukaryotic cell under conditions that promote expression of the fusion protein comprises contacting the cell with an agent that activates the inducible promoter. When the inducible promoter is an AOX1, DAK2, PEX11 promoter the agent that activates the inducible promoter is methanol.

### Glycoprotein and Sources Thereof

In some cases, the engineered eukaryotic cell that expresses the surface display fusion protein further comprises a genomic modification that overexpresses a secretory glycoprotein. Here, as a cell secretes the glycoprotein into the extracellular space, it comes in contact with a surface displayed fusion protein, which cleaves the oligosaccharide from the glycoprotein, with both the deglycosylated protein and the liberated oligosaccharide progressing into the extracellular space, e.g., the growth medium in which the eukaryotic cell is being cultured.

In alternate cases, a first engineered eukaryotic cell expresses the surface display fusion protein and a second engineered eukaryotic cell overexpresses a secretory glycoprotein. Here, the second cell secretes the glycoprotein into the extracellular space and it comes in contact with a surface displayed fusion protein on the first cell. The fusion protein cleaves the oligosaccharide from the glycoprotein, with both the deglycosylated protein and the liberated oligosaccharide progressing into the extracellular space, e.g., the growth medium in which the engineered eukaryotic cell is being cultured.

In other cases, a first engineered eukaryotic cell expresses the surface display fusion protein and further comprises a genomic modification that overexpresses a secretory glycoprotein, however, the fusion protein cleaves a secretory glycoprotein that was overexpressed by a second engineered eukaryotic cell.

The genomic modification that overexpresses a secretory glycoprotein may comprise a promoter (constitutive promoter, inducible promoter, and hybrid promoter) as disclosed herein; the genomic modification that overexpresses a secretory glycoprotein may comprise a terminator sequence as disclosed herein; the genomic modification that overexpresses a secretory glycoprotein may encode a secretory signal as disclosed herein; and/or the genomic modification that overexpresses a secretory glycoprotein may encode a signal sequence as disclosed herein.

A host cell may comprise a first promoter driving the expression of the fusion protein and a second promoter driving the expression secretory glycoprotein. The first and second promoter may be selected from the list of promoters provided herein. In some cases, the first promoter and the second promoter may be the same. Alternatively, the first and the second promoter may be different.

In various embodiments, the secreted glycoprotein is an animal protein. In some embodiments, the animal protein is an egg protein, e.g., selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

The glycoprotein may have amino acid sequence of any one of SEQ ID NO: 157 to SEQ ID NO: 290. The glycoprotein may be a variant of any one of SEQ ID NO: 157 to SEQ ID NO: 290. The variant may have at least or about 70%, 75%, 80%, 85%, 90%, 92%, 95%, 96%, 97%, 98%, 99%, or 100% sequence identity with one of SEQ ID NO: 157 to SEQ ID NO: 290.

Another aspect of the present disclosure is a population of engineered eukaryotic cells (that express a surface display fusion protein alone or that express a surface display fusion protein and overexpress a secretory glycoprotein) of any of the herein disclosed aspects or embodiment. The present disclosure further relates to a bioreactor comprising this population of engineered eukaryotic cells.

### Compositions

The present disclosure further relates to composition comprising any herein disclosed engineered eukaryotic cell, a secreted protein that has been deglycosylated, and one or more oligosaccharides cleaved from the secreted protein.

Also, the present disclosure further relates to a composition comprising a secreted protein that has been deglycosylated and one or more oligosaccharides cleaved from the secreted protein.

Further, the present disclosure relates to a composition comprising a secreted protein that has been deglycosylated.

Additionally, the present disclosure relates to a composition comprising one or more oligosaccharides cleaved from a secreted protein.

In various embodiments, the secreted glycoprotein is an animal protein. In some embodiments, the animal protein is an egg protein, e.g., selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

The glycoprotein may have amino acid sequence of any one of SEQ ID NO: 157 to SEQ ID NO: 290. The glycoprotein may be a variant of any one of SEQ ID NO: 157 to SEQ ID NO: 290. The variant may have at least or about 70%, 75%, 80%, 85%, 90%, 92%, 95%, 96%, 97%, 98%, 99%, or 100% sequence identity with one of SEQ ID NO: 157 to SEQ ID NO: 290.

These compositions may be liquid or dried. The secreted protein that has been deglycosylated and/or one or more oligosaccharides cleaved from the secreted protein may be lyophilized. In some cases, the secreted protein that has been deglycosylated and/or one or more oligosaccharides cleaved from the secreted protein are isolated, e.g., from each other and/or from a growth medium. The secreted protein that has been deglycosylated and/or one or more oligosaccharides cleaved from the secreted protein may be concentrated.

Deglycosylated proteins and/or one or more oligosaccharides cleaved from the secreted protein, as disclosed herein, may be used in a consumable composition comprising. Illustrative uses and features of such consumable compositions are described in WO 2016/077457, the contents of which is incorporated herein by reference in its entirety.

A consumable composition may comprise one or more deglycosylated proteins. As used herein, a consumable composition refers to a composition, which comprises an isolated deglycosylated protein and/or a cleaved oligosaccharide and may be consumed by an animal, including but not limited to humans and other mammals. Consumable food compositions include food products, beverage products, dietary supplements, food additives, and nutraceuticals as non-limiting examples. The consumable composition may comprise one or more components in addition to the deglycosylated protein. The one or more components may include ingredients, solvents used in the formation of foodstuff or beverages. For instance, the deglycosylated protein may be in the form of a powder which can be mixed with solvents to produce a beverage or mixed with other ingredients to form a food product.

The nutritional content of the deglycosylated protein may be higher than the nutritional content of an identical quantity of a control protein. The control protein may be the same protein produced recombinantly but not treated with a fusion protein of the present disclosure. The control protein may be the same protein produced recombinantly in a host cell which does not express a surface displayed fusion protein. The control protein may be the same protein isolated from a naturally occurring source. For instance, the control protein may be an isolated an egg white protein.

The nutritional content of a composition comprising the deglycosylated protein can be more than the nutritional content of the composition comprising a control protein. The protein content of the deglycosylated protein composition may be about 1% to 80% more than the protein content of a composition comprising a control protein. The protein content of the deglycosylated protein composition may be about 1% to 5% more than the protein content of a composition comprising a control protein. The protein content of the deglycosylated protein composition may be about 1% to 10% more than the protein content of a composition comprising a control protein. The protein content of the deglycosylated protein composition may be about 1% to 20% more than the protein content of a composition comprising a control protein. The protein content of the deglycosylated protein composition may be about 1% to 50% more than the protein content of a composition comprising a control protein. The protein content of the deglycosylated protein composition may be about 1% to 80% more than the protein content of a composition comprising a control protein. The protein content of the deglycosylated protein composition may be about 5% to 10%, 5-15%, 5-20%, 5-30%, 5-50%, 5-80% more than the protein content of a composition comprising a control protein. The protein content of the deglycosylated protein composition may be about 10% to 80%, 10-20%, 10-30%, 10-50%, 10-70%, 10-80% more than the protein content of a composition comprising a control protein. The protein content of the deglycosylated protein composition may be about 1%, 5%, 10%, 20%, 30%, 40%, 50%, 60%, 70%, or 80% more than the protein content of a composition comprising a control protein.

Protein content of a deglycosylated protein composition may be measured using conventional methods. For instance, protein content may be measured using nitrogen quantitation by combustion and then using a conversion factor to estimate quantity of protein in a sample followed by calculating the percentage (w/w) of the dry matter.

The nitrogen to carbon ratio of a deglycosylated protein be higher than the nitrogen to carbon ratio of a control protein. The nitrogen to carbon ratio of a recombinant protein may be greater than or equal to about 0.1. The nitrogen to carbon ratio of a deglycosylated protein be higher than the nitrogen to carbon ratio of a control protein. The nitrogen to carbon ratio of a recombinant protein may be greater than or equal to about 0.25. The nitrogen to carbon ratio of a recombinant protein may be greater than or equal to about 0.3. The nitrogen to carbon ratio of a recombinant protein may be greater than or equal to about 0.35. The nitrogen to carbon ratio of a recombinant protein may be greater than or equal to about 0.4. The nitrogen to carbon ratio of a recombinant protein may be greater than or equal to about 0.5.

Solubility of a deglycosylated protein may be greater than the solubility of a control protein. Solubility of a composition comprising a deglycosylated protein may be higher than the solubility of a composition comprising the control protein. Thermal stability of the deglycosylated protein may be greater than the thermal stability of a control protein.

The degree of glycosylation of the recombinant protein may be dependent on the consumable composition being produced. For instance, a consumable composition may comprise a lower degree of glycosylation to increase the protein content of the composition. Alternatively, the degree of glycosylation may be higher to increase the solubility of the protein in the composition.

### Methods for Deglycosylating a Secreted Protein

Another aspect of the present disclosure is a method for deglycosylating a secreted glycoprotein. The method comprises contacting a secreted protein with a fusion protein anchored to any herein-disclosed engineered eukaryotic cell. By contacting a secreted protein with the fusion protein, the catalytic domain cleaves and releases an oligonucleotide from the secreted glycoprotein.

In some cases, the secreted glycoprotein is expressed by the engineered eukaryotic cell.

Notably, a fusion protein anchored to an engineered eukaryotic cell (of the present disclosure) is more effective at deglycosylating the secreted glycoprotein than an intracellular endoglycosidase, e.g., an intracellular endoglycosidase located within a Golgi vesicle. In particular, a fusion protein anchored to the surface of an engineered eukaryotic cell (of the present disclosure) is more effective at deglycosylating the secreted glycoprotein than an intracellular endoglycosidase that is linked to a membrane associating domain, e.g., a membrane associating domain that comprises an amino acid sequence of OCH1. Preferably, the amino acid sequence of OCH1 that is included in a fusion protein of the present disclosure lacks the wild-type OCH1 Golgi retention domain. This retention domain comprises at least a portion of the first 48 residues of Pichia OCH1 protein. If the Golgi retention domain of OCH1 is included in a fusion protein of the present disclosure, then it is unlikely that the fusion protein would be displayed on the exterior of the cell, as needed to be a surface displayed fusion protein of the present disclosure. In embodiments, a fusion protein having an OCH1 anchoring domain lacks the OCH1 Golgi retention domain. In some embodiments, a fusion protein having an OCH1 anchoring domain lacks at least a portion of the first 48 residues of Pichia OCH1 protein. In various embodiments, a fusion protein having an OCH1 anchoring domain lacks the first 48 residues of Pichia OCH1 protein.

A deglycosylated protein of the present disclosure can have a level of N-linked glycosylation that is reduced by at least about 10 percent (e.g., 10 percent, 20 percent, 30 percent, 40 percent, 50 percent, 60 percent, 70 percent, 80 percent, 90 percent, or 100 percent) as compared to the level of N-linked glycosylation of the same glycoprotein that is not contacted with a fusion protein of the present disclosure, including a glycoprotein contacted with an intracellular endoglycosidase.

In some cases, the secreted glycoprotein is expressed by a cell other than the engineered eukaryotic cell.

In some embodiments, the method further comprises a step of isolating the deglycosylated secreted protein, e.g., from a cleaved oligosaccharide and/or from its growth medium. In some embodiments, the method further comprises a step of drying the deglycosylated secreted protein and/or the cleaved oligosaccharides.

In various embodiments, the secreted glycoprotein is an animal protein. In some embodiments, the animal protein is an egg protein, e.g., selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

The glycoprotein may have amino acid sequence of any one of SEQ ID NO: 157 to SEQ ID NO: 290. The glycoprotein may be a variant of any one of SEQ ID NO: 157 to SEQ ID NO: 290. The variant may have at least or about 70%, 75%, 80%, 85%, 90%, 92%, 95%, 96%, 97%, 98%, 99%, or 100% sequence identity with one of SEQ ID NO: 157 to SEQ ID NO: 290.

Another aspect of the present disclosure is a method for deglycosylating a plurality of secreted glycoproteins. The method comprises contacting the plurality of secreted glycoproteins with a population of any herein disclosed engineered eukaryotic cells. By contacting the plurality of secreted glycoprotein with the fusion protein, the catalytic domains cleave and release oligonucleotides from the plurality secreted glycoprotein and provide a plurality of deglycosylated secreted proteins.

In some cases, substantially every secreted glycoprotein in the plurality of secreted glycoproteins is deglycosylated upon contact with the population of engineered eukaryotic cells.

Notably, the amount of deglycosylation of the secreted glycoproteins is not increased by further contacting the secreted protein with an isolated endoglycosidase.

Further, the amount of deglycosylation of the secreted glycoproteins is more than the amount obtained from a population of cells that express an intracellular endoglycosidase in addition to expressing the secreted glycoprotein.

In some embodiments, the method further comprises a step of isolating the plurality of deglycosylated secreted proteins and may further comprise a step of drying the plurality of deglycosylated secreted proteins.

In various embodiments, the secreted glycoprotein is an animal protein. In some embodiments, the animal protein is an egg protein, e.g., selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

The glycoprotein may have amino acid sequence of any one of SEQ ID NO: 157 to SEQ ID NO: 290. The glycoprotein may be a variant of any one of SEQ ID NO: 157 to SEQ ID NO: 290. The variant may have at least or about 70%, 75%, 80%, 85%, 90%, 92%, 95%, 96%, 97%, 98%, 99%, or 100% sequence identity with one of SEQ ID NO: 157 to SEQ ID NO: 290.

### Additional Catalytic Domains

Much of the above disclosure relates to surface displayed fusion proteins comprising a catalytic domain of an endoglycosidase, e.g., endoglycosidase H.

The engineered cells, nucleic acid sequences, compositions, and method disclosed herein may be adapted to relate to fusion proteins with catalytic domains of enzymes other than endoglycosidases. As used herein, the term “catalytic domain” comprises a portion of an enzyme that provides catalytic activity.

Accordingly, another aspect of the present disclosure is an engineered eukaryotic cell which expresses a surface displayed catalytic domain of an enzyme, wherein the catalytic domain is directly or indirectly tethered to the exterior surface of the cell.

Any aspect or embodiment described herein can be combined with any other aspect or embodiment as disclosed herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

The section headings used herein are for organizational purposes only and are not to be construed as limiting the subject matter described.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

The following examples are included for illustrative purposes only and are not intended to limit the scope of the invention.

### Example 1: Construction of a Surface Displayed EndoH-Sed1p Fusion Protein

A nucleic acid sequence that expressed a surface displayed fusion protein of SEQ ID NO: 10 was constructed and transfected in to Pichia cells. Transfected cells that faithfully expressed and surface displayed the fusion protein were isolated and expanded in culture.

The fusion protein included the Saccharomyces cerevisiae alpha mating factor signal peptide and secretion signal (89 residues, ending in EAEA; SEQ ID NO: 21), EndoH codon variant 2 (271 residues; SEQ ID NO: 1), a flex linker of 26 residues [GSS]₈ (eight repeats of SEQ ID NO: 23), a semi-rigid alpha helix linker of 20 residues [EAAAR]₄, (SEQ ID NO: 24) another flex linker of 15 residues [GGGGS]₃ (three repeats of SEQ ID NO: 22) and the full Sed1 gene minus the N term 18 amino acid signal peptide (320 residues; SEQ ID NO: 3). Glycine-Serine linkers are commonly used in fusion proteins to space them out with no intervening secondary structure. The ratio of serine to glycine determines the relative stiffness of the linker, but even high serine content GS linkers are still fairly flexible. The entire linker of this fusion protein has an amino acid sequence of SEQ ID NO: 25. The full fusion protein had the amino acid sequence of SEQ ID NO: 10.

During translation and processing by the engineered cell, the signal peptide (MRFPSIFTAVLFAASSALA; SEQ ID NO: 59) was first cleaved off in the cell's endoplasmic reticulum. When the protein arrives in the late Golgi, the secretion signal (APVNTTTEDETAQIPAEAVIGYSDLEGDFDVAVLPFSNSTNNGLLFINTTIASIAAKEEGV SLDKR; SEQ ID NO: 291) was cleaved off. Around the same time, the propeptide on the C-term (APVNTTTEDETAQIPAEAVIGYSDLEGDFDVAVLPFSNSTNNGLLFINTTIASIAAKEEGV SLDKREAEA; SEQ ID NO: 292) was also cleaved off for the attachment of the GPI anchor. The final resultant fusion protein is as below, and include the full EndoH protein, the mature Sed1 protein, plus various linker elements and having the amino acid sequence of SEQ ID NO: 9.

The surface displayed fusion protein was incorporated into the cell membrane via a GPI anchor attached to the protein's C-terminus.

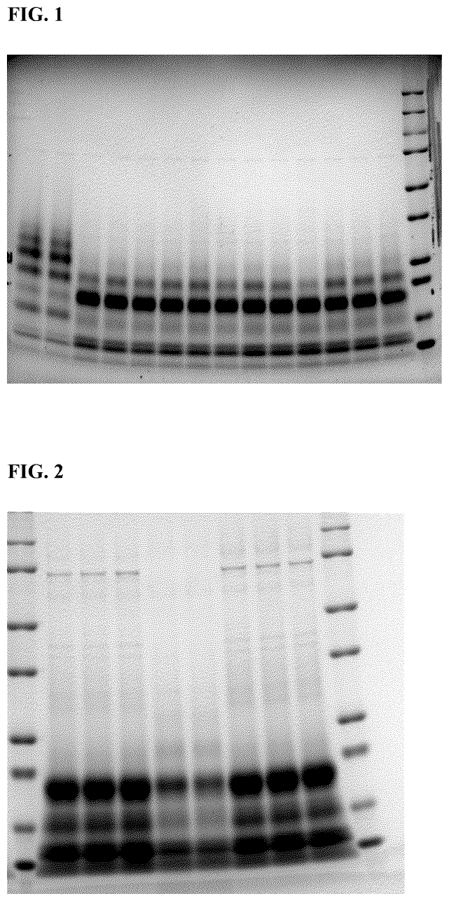

This surface displayed fusion protein was shown to be effective at deglycosylating an illustrative secreted glycoprotein (here, ovomucoid (OVD)). A high-throughput screen of cells engineered cells to express OVD and the surface displayed EndoH-Sed1p fusion protein was performed. In this screen, all engineered cell lines were capable of fully deglycosylating OVD while maintaining OVD titer. As shown in FIG. 1, secreted OVD absent the fusion protein comprises heavy glycosylated species (left two lanes), whereas engineered cells expressing the EndoH-Sed1p fusion protein cleaved off the glycoprotein's oligosaccharides, leaving a lighter, deglycosylated protein bands.

To expand production of EndoH-Sed1p fusion protein/glycoprotein secreting P. pastoris cells, a seed strain was removed from cryo-storage and thawed to room temperature. Contents of the thawed seed vials were used to inoculate liquid seed culture media in baffled flasks which were grown at 30° C. in shaking incubators. These seed flasks were then transferred and grown in a series of larger and larger seed fermenters containing a basal salt media, trace metals, and glucose. The temperature in the seed reactors were controlled at 30° C., pH at 5, and dissolved oxygen (DO) at 30%. pH was maintained by feeding ammonia hydroxide which also acted as a nitrogen source. Once sufficient cell mass was reached, the grown EndoH-Sed1p fusion protein/glycoprotein secreting P. pastoris was inoculated in a production-scale reactor containing basal salt media, trace metals, and glucose. Like in the seed tanks, the culture was also controlled at 30° C., pH 5 and 30% DO throughout the process. pH was again maintained by feeding ammonia hydroxide. During the initial batch glucose phase, the culture was left to consume all glucose and subsequently-produced ethanol. Once the target cell density was achieved and glucose and ethanol concentrations were confirmed to be zero, the glucose fed-batch growth phase was initiated. In this phase, glucose was fed until the culture reaches a target cell density. Glucose was fed at a limiting rate to prevent ethanol from building up in the presence of non-zero glucose concentrations. In the final induction phase, the culture was co-fed glucose and methanol which induced the cells to produce EndoH-Sed1p fusion protein via a methanol-inducible promoter included in the construct expressing the fusion protein. Glucose was fed at an amount to produce a desired growth rate, while methanol was fed to maintain the methanol concentration at 1% to ensure that fusion protein expression was consistently induced. Regular samples were taken throughout the fermentation process for analyses of specific process parameters (e.g., cell density, glucose/methanol concentrations, product titer, and quality).

The bioreactor-expanded cells were assayed for their ability to deglycosylate an illustrative glycoprotein. As shown in FIG. 2, in bioreactor cultures, engineered cells expressing the EndoH-Sed1p fusion protein cleaved off the glycoprotein's oligosaccharides, leaving faster migrating, deglycosylated protein bands.

Another version of the surface displayed fusion protein described above was generated with a shorter linker (i.e., [GGGGS]₃) and with a different EndoH codon set. Surprisingly, this other version of the fusion protein has much lower deglycosylation ability.

### Example 2: Construction of a Surface Displayed EndoH—Flo5-2 Fusion Protein

A nucleic acid sequence that expressed a surface displayed fusion protein of SEQ ID NO: 12 was constructed and transfected into Pichia cells. Transfected cells that faithfully expressed and surface displayed the fusion protein were isolated and expanded in culture.

Overexpression results in Pichia cells showed that Flo5-2 strongly flocculates pichia cells. These results were conducted in cells that did not co-express a secreted glycoprotein and had low exopolysaccharides.

The EndoH—Flo5-2 fusion protein was designed to take advantage of Flo5-2's ability to flocculate pichia cells and endoH's ability to cleave off oligosaccharides from glycoproteins. Without wishing to be bound by theory, the endoH on the N terminal end of the fusion protein should shield the Flo5-2 protein and reduce the risk of flocculation while giving enough space (via linkers) for exopolysaccharides present in the extracellular space be captured. Flo proteins naturally extend well into the extracellular space because they need to be able to adhere to cell wall of another cell. Therefore, combining EndoH with Flo5-2 would provide an extended reach for the enzyme to bind to and cleave secreted glycoproteins present in the extracellular space.

The surface displayed EndoH—Flo5-2 fusion protein had the following structure: a Flo5-2 signal peptide (MKFPVPLLFLLQLFFIIATQG; SEQ ID NO: 61), EndoH (SEQ ID NO: 1), a complex linker (SEQ ID NO: 25), and a Flo5-2 mature protein (SEQ ID NO: 5) plus the propeptide that gets cut off for GPI anchoring. The propeptide that's cleaved off within the cell is on Flo5-2's the C-terminal and is likely around the same size as Sed1's propeptide of about 20 amino acids.

The surface displayed EndoH—Flo5-2 fusion protein uses Flo5-2's native signal peptide. Flo5-2 secretes itself without needing another secretion signal. So, this fusion protein did not include an alpha factor secretion signal, as used in the EndoH-Sed1 fusion protein. However, adding an alpha factor secretion signal is considered and may improve secretion of the fusion protein.

In a high throughput screen, surface displayed EndoH— Flo5-2 fusion protein was capable of fully deglycosylating an illustrative co-expressed glycoprotein (here, OVD) and at a fairly high rate.

### Example 3: Construction of a Surface Displayed EndoH—Saccharomyces cerevisiae Flo5 Fusion Protein

A nucleic acid sequence that expressed a surface displayed fusion protein of SEQ ID NO: 293 was constructed and transfected into Pichia cells. Transfected cells that faithfully expressed and surface displayed the fusion protein were isolated and expanded in culture.

A high throughput screen showed that the surface displayed EndoH—Saccharomyces cerevisiae Flo5 fusion protein fully deglycosylated an illustrative co-expressed glycoprotein (here, OVD).

### Example 4: Construction of a Surface Displayed EndoH-Flo11 Fusion Protein

A nucleic acid sequence that expressed a surface displayed fusion protein of SEQ ID NO: 14 are constructed and are transfected into Pichia cells. Transfected cells that faithfully express and surface display the fusion protein will be isolated and expanded in culture. And the fusion protein's ability to fully deglycosylated an illustrative co-expressed glycoprotein will be assayed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1
Sequences

| | | |
|---|---|---|
| mature EndoH seq | SEQ ID NO: 1 | APAPVKQGPTSVAYVEVNNNSMLNVGKYTLADGGGNAFDV |
| only without | | AVIFAANINYDTGTKTAYLHFNENVQRVLDNAVTQIRPLQ |
| its native | | QQGIKVLLSVLGNHQGAGFANFPSQQAASAFAKQLSDAVA |
| signal peptide | | KYGLDGVDFDDEYAEYGNNGTAQPNDSSFVHLVTALRANM |
| | | PDKIISLYNIGPAASRLSYGGVDVSDKFDYAWNPYYGTWQ |
| | | VPGIALPKAQLSPAAVEIGRTSRSTVADLARRTVDEGYGV |
| | | YLTYNLDGGDRTADVSAFTRELYGSEAVRTP |
| endoH | SEQ ID NO: 2 | MFTPVRRRVRTAALALSAAAALVLGSTAASGASATPSPAP |
| (with signal peptide | | APAPAPVKQGPTSVAYVEVNNNSMLNVGKYTLADGGGNAF |
| underlined) | | DVAVIFAANINYDTGTKTAYLHFNENVQRVLDNAVTQIRP |
| | | LQQQGIKVLLSVLGNHQGAGFANFPSQQAASAFAKQLSDA |
| | | VAKYGLDGVDFDDEYAEYGNNGTAQPNDSSFVHLVTALRA |
| | | NMPDKIISLYNIGPAASRLSYGGVDVSDKFDYAWNPYYGT |
| | | WQVPGIALPKAQLSPAAVEIGRTSRSTVADLARRTVDEGY |
| | | GVYLTYNLDGGDRTADVSAFTRELYGSEAVRTP |
| Sed1 from | SEQ ID NO: 3 | QFSNSTSASSTDVTSSSSISTSSGSVTITSSEAPESDNGT |
| Saccharomyces | | STAAPTETSTEAPTTAIPTNGTSTEAPTTAIPTNGTSTEA |
| cerevisiae | | PTDTTTEAPTTALPTNGTSTEAPTDTTTEAPTTGLPTNGT |
| | | TSAFPPTTSLPPSNTTTTPPYNPSTDYTTDYTVVTEYTTY |
| | | CPEPTTFTTNGKTYTVTEPTTLTITDCPCTIEKPTTTSTT |
| | | EYTVVTEYTTYCPEPTTFTTNGKTYTVTEPTTLTITDCPC |
| | | TIEKSEAPESSVPVTESKGTTTKETGVTTKQTTANPSLTV |
| | | STVVPVSSSASSHSVVINSNGANVVVPGALGLAGVAMLFL |
| Sed1 from | SEQ ID NO: 4 | MKLSTVLLSAGLASTTLAQFSNSTSASSTDVTSSSSISTS |
| Saccharomyces | | SGSVTITSSEAPESDNGTSTAAPTETSTEAPTTAIPTNGT |
| cerevisiae | | STEAPTTAIPTNGTSTEAPTDTTTEAPTTALPTNGTSTEA |
| (underlined | | PTDTTTEAPTTGLPTNGTTSAFPPTTSLPPSNTTTTPPYN |
| is signal peptide, not | | PSTDYTTDYTVVTEYTTYCPEPTTFTTNGKTYTVTEPTTL |
| utilized in design) | | TITDCPCTIEKPTTTSTTEYTVVTEYTTYCPEPTTFTTNG |
| | | KTYTVTEPTTLTITDCPCTIEKSEAPESSVPVTESKGTTT |
| | | KETGVTTKQTTANPSLTVSTVVPVSSSASSHSVVINSNGA |
| | | NVVVPGALGLAGVAMLFL |
| Flo5-2 from | SEQ ID NO: 5 | DESGNGDESDTAYGCDITSNAFDGFDATIYEYNANDLKLI |
| Komagataella phaffii | | RDPVFMSTGYLGRNVLNKISGVTVPGFNIWNPRSRTATVY |
| | | GVQNVNYYNMVLELKGYFKAAVSGDYKLTLSNIDDSSMLF |
| | | FGKNTAFQCCDTGSIPVDQAPTDYSLFTIKPSNQVNSEVI |
| | | SSTQYLEAGKYYPVRIVFVNALERALFNFKLTIPSGTVLD |
| | | DFQDYIYQFGALDENSCYETTVSKITEWTTYTTPWTGTFE |
| | | TTRTITPTGTEGTVVIETPESYVTTTQPWTGTYETTYTVP |
| | | PTGTEPGTVIIETPEIIDCEAVCCGPFLTAFSFRKREECQ |
| | | CENICCPGDTNCETYVTTTQPWTGTYETTYTVPPTGTEPG |
| | | TVIIETPESYVTTTQPWTGTYETTYTVPPTGTEPGTVIIE |
| | | TPESYVTTTQPWTGTYETTYTVPPSGTEPGTVVIETPEIV |
| | | DCEAYCCASVAIKKRELCQCENFCCSWDQSCQTYVTTTQP |
| | | WTGTYETTYTVPPTGTEPGTVIIETPESYVTTTQPWTGTY |
| | | ETTYTVPPTGTEPGTVIIETPESYVTTTQPWTGTYETTYT |
| | | VPPTGTEPGTVIIETPEIIDCEAVCCGPFLTAFSFRKREE |
| | | CQCENICCPGDTNCETYVTTTQPWTGTYETTYTVPPTGTE |
| | | PGTVIIETPESYVTTTQPWTGTYETTYTVPPTGTEPGTVI |
| | | IETPESYVTTTQPWTGTYETTYTVPPTGTEPGTVIIETPE |
| | | IINCEAVCCGPFLTAFSFRKREECQCENICCPGDTNCETY |
| | | VTTTQPWTGTYETTYTVPPTGTEPGTVIIETPESYVTTTQ |
| | | PWTGTYETTYTVPSTGTEPGTVIIETPESYVTTTQPWTGT |
| | | YETTFTVPPTGTEPGTVVIETPESYVTTTQPWTGTYETTY |
| | | SVPPSGTEPGTVVIETPESYVTTTQPWTGTYETTYSVPPS |
| | | GTEPGTVVIETPEASTARTKFTTVTSSWTGVFTTTKTLPA |
| | | SGTEPATIVIQTPTGYFNTSSLVSTRTKTNVDTVTRVIPC |
| | | PICTAPKTITVVPEEPNESVSVIISQPQSSSTDTTLSKPD |
| | | SVRVISQPETASQMDTSLSKTDSAVISTETAGNNIIPLAG |
| | | SHSYNTIVTTVTDSPQVAQSTTATSSSNVHLTISTQTTTP |
| | | SLVYSSSLSTVHQVSPSNGGFRSSITVHPLLSVIGAIFGA |
| | | LFM |
| Flo5-2 from | SEQ ID NO: 6 | MKFPVPLLFLLQLFFIIATQGDESGNGDESDTAYGCDITS |
| Komagataella phaffii | | NAFDGFDATIYEYNANDLKLIRDPVFMSTGYLGRNVLNKI |
| (underlined is signal | | SGVTVPGFNIWNPRSRTATVYGVQNVNYYNMVLELKGYFK |
| peptide, used in some | | AAVSGDYKLTLSNIDDSSMLFFGKNTAFQCCDTGSIPVDQ |
| versions and not | | APTDYSLFTIKPSNQVNSEVISSTQYLEAGKYYPVRIVFV |
| others) | | NALERALFNFKLTIPSGTVLDDFQDYIYQFGALDENSCYE |
| | | TTVSKITEWTTYTTPWTGTFETTRTITPTGTEGTVVIETP |
| | | ESYVTTTQPWTGTYETTYTVPPTGTEPGTVIIETPEIIDC |
| | | EAVCCGPFLTAFSFRKREECQCENICCPGDTNCETYVTTT |
| | | QPWTGTYETTYTVPPTGTEPGTVIIETPESYVTTTQPWTG |
| | | TYETTYTVPPTGTEPGTVIIETPESYVTTTQPWTGTYETT |
| | | YTVPPSGTEPGTVVIETPEIVDCEAYCCASVAIKKRELCQ |
| | | CENFCCSWDQSCQTYVTTTQPWTGTYETTYTVPPTGTEPG |
| | | TVIIETPESYVTTTQPWTGTYETTYTVPPTGTEPGTVIIE |
| | | TPESYVTTTQPWTGTYETTYTVPPTGTEPGTVIIETPEII |
| | | DCEAVCCGPFLTAFSFRKREECQCENICCPGDTNCETYVT |
| | | TTQPWTGTYETTYTVPPTGTEPGTVIIETPESYVTTTQPW |
| | | TGTYETTYTVPPTGTEPGTVIIETPESYVTTTQPWTGTYE |
| | | TTYTVPPTGTEPGTVIIETPEIINCEAVCCGPFLTAFSFR |
| | | KREECQCENICCPGDTNCETYVTTTQPWTGTYETTYTVPP |
| | | TGTEPGTVIIETPESYVTTTQPWTGTYETTYTVPSTGTEP |
| | | GTVIIETPESYVTTTQPWTGTYETTFTVPPTGTEPGTVVI |
| | | ETPESYVTTTQPWTGTYETTYSVPPSGTEPGTVVIETPES |
| | | YVTTTQPWTGTYETTYSVPPSGTEPGTVVIETPEASTART |
| | | KFTTVTSSWTGVFTTTKTLPASGTEPATIVIQTPTGYFNT |
| | | SSLVSTRTKTNVDTVTRVIPCPICTAPKTITVVPEEPNES |
| | | VSVIISQPQSSSTDTTLSKPDSVRVISQPETASQMDTSLS |
| | | KTDSAVISTETAGNNIIPLAGSHSYNTIVTTVTDSPQVAQ |
| | | STTATSSSNVHLTISTQTTTPSLVYSSSLSTVHQVSPSNG |
| | | GFRSSITVHPLLSVIGAIFGALFM |
| Flo11 from | SEQ ID NO: 7 | SSGKTCPTSEVSPACYANQWETTFPPSDIKITGATWVQDN |
| Komagataella phaffii | | IYDVTLSYEAESLELENLTELKIIGLNSPTGGTKLVWSLN |
| (no signal sequence) | | SKVYDIDNPAKWTTTLRVYTKSSADDCYVEMYPFQIQVDW |
| | | CEAGASTDGCSAWKWPKSYDYDIGCDNMQDGVSRKHHPVY |
| | | KWPKKCSSNCGVEPTTSDEPEEPTTSEEPEEPTTSEEPEE |
| | | PTSSDEEPTTSEEPEEPTTSDEPEEPTTSEEPEEPTTSEE |
| | | PEEPTTSEEPTTSEEPEEPTSSDEEPTTSDEPEEPTTSDE |
| | | PEEPTTSEEPTTSEEPEEPTTSSEEPTPSEEPEGPTCPTS |
| | | EVSPACYADQWETTFPPSDIKITGATWVEDNIYDVTLSYE |
| | | AESLELENLTELKIIGLNSPTGGTKVVWSLNSGIYDIDNP |
| | | AKWTTTLRVYTKSSADDCYVEMYPFQIQVDWCEAGASTDG |
| | | CSAWKWPKSYDYDIGCDNMQDGVSRKHHPVYKWPKKCSSD |
| | | CGVEPTTSDEPEEPTTSEEPVEPTSSDEEPTTSEEPTTSE |
| | | EPEEPTTSDEPEEPTTSEEPEEPTTSEEPEEPTTSEEPTT |
| | | SEEPEEPTSSDEEPTTSDEPEEPTTSEEPEEPTTSEEPEE |
| | | PTTSEEPEEPTTSDEPEEPTTSEEPEEPTTSEEPEEPTSS |
| | | DEEPTTSEEPEEPTTSEEPEEPTTSEEPEEPTTSEEPEEP |
| | | TSSDEEPTTSEEPEEPTTSDEPEEPTTSEEPEEPTTSEEP |
| | | EEPTSSDEEPTTSEEPEEPTTSDEPEEPTTSEEPEEPTTS |
| | | EEPEEPTTSEEPEEPTTSEEPEEPTSSDEEPTTSEEPEEP |
| | | TTSDEPEEPTTSEEPEEPTTSEEPEEPTTSEEPEEPTTSD |
| | | EEPGTTEEPLVPTTKTETDVSTTLLTVTDCGTKTCTKSLV |
| | | ITGVTKETVTTHGKTTVITTYCPLPTETVTPTPVTVTSTI |
| | | YADESVTKTTVYTTGAVEKTVTVGGSSTVVVVHTPLTTAV |
| | | VQSQSTDEIKTVVTARPSTTTIVRDVCYNSVCSVATIVTG |
| | | VTEKTITFSTGSITVVPTYVPLVESEEHQRTASTSETRAT |
| | | SVVVPTVVGQSSSASATSSIFPSVTIHEGVANTVKNSMIS |
| | | GAVALLFNALFL |
| Flo11 from | SEQ ID NO: 8 | MVSLRSIFTSSILAAGLTRAHGSSGKTCPTSEVSPACYAN |
| Komagataella phaffii | | QWETTFPPSDIKITGATWVQDNIYDVTLSYEAESLELENL |
| (with signal sequence) | | TELKIIGLNSPTGGTKLVWSLNSKVYDIDNPAKWTTTLRV |
| | | YTKSSADDCYVEMYPFQIQVDWCEAGASTDGCSAWKWPKS |
| | | YDYDIGCDNMQDGVSRKHHPVYKWPKKCSSNCGVEPTTSD |
| | | EPEEPTTSEEPEEPTTSEEPEEPTSSDEEPTTSEEPEEPT |
| | | TSDEPEEPTTSEEPEEPTTSEEPEEPTTSEEPTTSEEPEE |
| | | PTSSDEEPTTSDEPEEPTTSDEPEEPTTSEEPTTSEEPEE |
| | | PTTSSEEPTPSEEPEGPTCPTSEVSPACYADQWETTFPPS |
| | | DIKITGATWVEDNIYDVTLSYEAESLELENLTELKIIGLN |
| | | SPTGGTKVVWSLNSGIYDIDNPAKWTTTLRVYTKSSADDC |
| | | YVEMYPFQIQVDWCEAGASTDGCSAWKWPKSYDYDIGCDN |
| | | MQDGVSRKHHPVYKWPKKCSSDCGVEPTTSDEPEEPTTSE |
| | | EPVEPTSSDEEPTTSEEPTTSEEPEEPTTSDEPEEPTTSE |
| | | EPEEPTTSEEPEEPTTSEEPTTSEEPEEPTSSDEEPTTSD |
| | | EPEEPTTSEEPEEPTTSEEPEEPTTSEEPEEPTTSDEPEE |
| | | PTTSEEPEEPTTSEEPEEPTSSDEEPTTSEEPEEPTTSEE |
| | | PEEPTTSEEPEEPTTSEEPEEPTSSDEEPTTSEEPEEPTT |
| | | SDEPEEPTTSEEPEEPTTSEEPEEPTSSDEEPTTSEEPEE |
| | | PTTSDEPEEPTTSEEPEEPTTSEEPEEPTTSEEPEEPTTS |
| | | EEPEEPTSSDEEPTTSEEPEEPTTSDEPEEPTTSEEPEEP |
| | | TTSEEPEEPTTSEEPEEPTTSDEEPGTTEEPLVPTTKTET |
| | | DVSTTLLTVTDCGTKTCTKSLVITGVTKETVTTHGKTTVI |
| | | TTYCPLPTETVTPTPVTVTSTIYADESVTKTTVYTTGAVE |
| | | KTVTVGGSSTVVVVHTPLTTAVVQSQSTDEIKTVVTARPS |
| | | TTTIVRDVCYNSVCSVATIVTGVTEKTITFSTGSITVVPT |
| | | YVPLVESEEHQRTASTSETRATSVVVPTVVGQSSSASATS |
| | | SIFPSVTIHEGVANTVKNSMISGAVALLFNALFL |
| EndoH-Sed1 fusion | SEQ ID NO: 9 | EAEAAPAPVKQGPTSVAYVEVNNNSMLNVGKYTLADGGGN |
| (partial ORF, without | | AFDVAVIFAANINYDTGTKTAYLHFNENVQRVLDNAVTQI |
| peptides that are | | RPLQQQGIKVLLSVLGNHQGAGFANFPSQQAASAFAKQLS |
| cleaved off post- | | DAVAKYGLDGVDFDDEYAEYGNNGTAQPNDSSFVHLVTAL |
| translationally) | | RANMPDKIISLYNIGPAASRLSYGGVDVSDKFDYAWNPYY |
| | | GTWQVPGIALPKAQLSPAAVEIGRTSRSTVADLARRTVDE |
| | | GYGVYLTYNLDGGDRTADVSAFTRELYGSEAVRTPGSSGS |
| | | SGSSGSSGSSGSSGSSGSSEAAAREAAAREAAAREAAARG |
| | | GGGSGGGGSGGGGSQFSNSTSASSTDVTSSSSISTSSGSV |
| | | TITSSEAPESDNGTSTAAPTETSTEAPTTAIPTNGTSTEA |
| | | PTTAIPTNGTSTEAPTDTTTEAPTTALPTNGTSTEAPTDT |
| | | TTEAPTTGLPTNGTTSAFPPTTSLPPSNTTTTPPYNPSTD |
| | | YTTDYTVVTEYTTYCPEPTTFTTNGKTYTVTEPTTLTITD |
| | | CPCTIEKPTTTSTTEYTVVTEYTTYCPEPTTFTTNGKTYT |
| | | VTEPTTLTITDCPCTIEKSEAPESSVPVTESKGTTTKETG |
| | | VTTKQTTANPSLTVSTVVPVSSSASSHSVVINSN |
| EndoH-Sedl fusion | SEQ ID NO: 10 | MRFPSIFTAVLFAASSALAAPVNTTTEDETAQIPAEAVIG |
| (full ORF, including | | YSDLEGDFDVAVLPFSNSTNNGLLFINTTIASIAAKEEGV |
| peptides that are | | SLDKREAEAAPAPVKQGPTSVAYVEVNNNSMLNVGKYTLA |
| cleaved off post- | | DGGGNAFDVAVIFAANINYDTGTKTAYLHFNENVQRVLDN |
| translationally) | | AVTQIRPLQQQGIKVLLSVLGNHQGAGFANFPSQQAASAF |
| | | AKQLSDAVAKYGLDGVDFDDEYAEYGNNGTAQPNDSSFVH |
| | | LVTALRANMPDKIISLYNIGPAASRLSYGGVDVSDKFDYA |
| | | WNPYYGTWQVPGIALPKAQLSPAAVEIGRTSRSTVADLAR |
| | | RTVDEGYGVYLTYNLDGGDRTADVSAFTRELYGSEAVRTP |
| | | GSSGSSGSSGSSGSSGSSGSSGSSEAAAREAAAREAAARE |
| | | AAARGGGGSGGGGSGGGGSQFSNSTSASSTDVTSSSSIST |
| | | SSGSVTITSSEAPESDNGTSTAAPTETSTEAPTTAIPTNG |
| | | TSTEAPTTAIPTNGTSTEAPTDTTTEAPTTALPTNGTSTE |
| | | APTDTTTEAPTTGLPTNGTTSAFPPTTSLPPSNTTTTPPY |
| | | NPSTDYTTDYTVVTEYTTYCPEPTTFTTNGKTYTVTEPTT |
| | | LTITDCPCTIEKPTTTSTTEYTVVTEYTTYCPEPTTFTTN |
| | | GKTYTVTEPTTLTITDCPCTIEKSEAPESSVPVTESKGTT |
| | | TKETGVTTKQTTANPSLTVSTVVPVSSSASSHSVVINSNG |
| | | ANVVVPGALGLAGVAMLFL |
| EndoH-Flo5-2 fusion | SEQ ID NO: 11 | APAPVKQGPTSVAYVEVNNNSMLNVGKYTLADGGGNAFDV |
| (partial ORF, without | | AVIFAANINYDTGTKTAYLHFNENVQRVLDNAVTQIRPLQ |
| signal peptide that is | | QQGIKVLLSVLGNHQGAGFANFPSQQAASAFAKQLSDAVA |
| cleaved off post- | | KYGLDGVDFDDEYAEYGNNGTAQPNDSSFVHLVTALRANM |
| translationally) | | PDKIISLYNIGPAASRLSYGGVDVSDKFDYAWNPYYGTWQ |
| | | VPGIALPKAQLSPAAVEIGRTSRSTVADLARRTVDEGYGV |
| | | YLTYNLDGGDRTADVSAFTRELYGSEAVRTPGSSGSSGSS |
| | | GSSGSSGSSGSSGSSEAAAREAAAREAAAREAAARGGGGS |
| | | GGGGSGGGGSDESGNGDESDTAYGCDITSNAFDGFDATIY |
| | | EYNANDLKLIRDPVFMSTGYLGRNVLNKISGVTVPGFNIW |
| | | NPRSRTATVYGVQNVNYYNMVLELKGYFKAAVSGDYKLTL |
| | | SNIDDSSMLFFGKNTAFQCCDTGSIPVDQAPTDYSLFTIK |
| | | PSNQVNSEVISSTQYLEAGKYYPVRIVFVNALERALFNFK |
| | | LTIPSGTVLDDFQDYIYQFGALDENSCYETTVSKITEWTT |
| | | YTTPWTGTFETTRTITPTGTEGTVVIETPESYVTTTQPWT |
| | | GTYETTYTVPPTGTEPGTVIIETPEIIDCEAVCCGPFLTA |
| | | FSFRKREECQCENICCPGDTNCETYVTTTQPWTGTYETTY |
| | | TVPPTGTEPGTVIIETPESYVTTTQPWTGTYETTYTVPPT |
| | | GTEPGTVIIETPESYVTTTQPWTGTYETTYTVPPSGTEPG |
| | | TVVIETPEIVDCEAYCCASVAIKKRELCQCENFCCSWDQS |
| | | CQTYVTTTQPWTGTYETTYTVPPTGTEPGTVIIETPESYV |
| | | TTTQPWTGTYETTYTVPPTGTEPGTVIIETPESYVTTTQP |
| | | WTGTYETTYTVPPTGTEPGTVIIETPEIIDCEAVCCGPFL |
| | | TAFSFRKREECQCENICCPGDTNCETYVTTTQPWTGTYET |
| | | TYTVPPTGTEPGTVIIETPESYVTTTQPWTGTYETTYTVP |
| | | PTGTEPGTVIIETPESYVTTTQPWTGTYETTYTVPPTGTE |
| | | PGTVIIETPEIINCEAVCCGPFLTAFSFRKREECQCENIC |
| | | CPGDTNCETYVTTTQPWTGTYETTYTVPPTGTEPGTVIIE |
| | | TPESYVTTTQPWTGTYETTYTVPSTGTEPGTVIIETPESY |
| | | VTTTQPWTGTYETTFTVPPTGTEPGTVVIETPESYVTTTQ |
| | | PWTGTYETTYSVPPSGTEPGTVVIETPESYVTTTQPWTGT |
| | | YETTYSVPPSGTEPGTVVIETPEASTARTKFTTVTSSWTG |
| | | VFTTTKTLPASGTEPATIVIQTPTGYFNTSSLVSTRTKTN |
| | | VDTVTRVIPCPICTAPKTITVVPEEPNESVSVIISQPQSS |
| | | STDTTLSKPDSVRVISQPETASQMDTSLSKTDSAVISTET |
| | | AGNNIIPLAGSHSYNTIVTTVTDSPQVAQSTTATSSSNVH |
| | | LTISTQTTTPSLVYSSSLSTVHQVSPSNGGFRSSITVHPL |
| | | LSVIGAIFGALFM |
| EndoH-Flo5-2 fusion | SEQ ID NO: 12 | MKFPVPLLFLLQLFFIIATQGAPAPVKQGPTSVAYVEVNN |
| (full ORF, including | | NSMLNVGKYTLADGGGNAFDVAVIFAANINYDTGTKTAYL |
| signal peptide that is | | HFNENVQRVLDNAVTQIRPLQQQGIKVLLSVLGNHQGAGF |
| cleaved off post- | | ANFPSQQAASAFAKQLSDAVAKYGLDGVDFDDEYAEYGNN |
| translationally) | | GTAQPNDSSFVHLVTALRANMPDKIISLYNIGPAASRLSY |
| | | GGVDVSDKFDYAWNPYYGTWQVPGIALPKAQLSPAAVEIG |
| | | RTSRSTVADLARRTVDEGYGVYLTYNLDGGDRTADVSAFT |
| | | RELYGSEAVRTPGSSGSSGSSGSSGSSGSSGSSGSSEAAA |
| | | REAAAREAAAREAAARGGGGSGGGGSGGGGSDESGNGDES |
| | | DTAYGCDITSNAFDGFDATIYEYNANDLKLIRDPVFMSTG |
| | | YLGRNVLNKISGVTVPGFNIWNPRSRTATVYGVQNVNYYN |
| | | MVLELKGYFKAAVSGDYKLTLSNIDDSSMLFFGKNTAFQC |
| | | CDTGSIPVDQAPTDYSLFTIKPSNQVNSEVISSTQYLEAG |
| | | KYYPVRIVFVNALERALFNFKLTIPSGTVLDDFQDYIYQF |
| | | GALDENSCYETTVSKITEWTTYTTPWTGTFETTRTITPTG |
| | | TEGTVVIETPESYVTTTQPWTGTYETTYTVPPTGTEPGTV |
| | | IIETPEIIDCEAVCCGPFLTAFSFRKREECQCENICCPGD |
| | | TNCETYVTTTQPWTGTYETTYTVPPTGTEPGTVIIETPES |
| | | YVTTTQPWTGTYETTYTVPPTGTEPGTVIIETPESYVTTT |
| | | QPWTGTYETTYTVPPSGTEPGTVVIETPEIVDCEAYCCAS |
| | | VAIKKRELCQCENFCCSWDQSCQTYVTTTQPWTGTYETTY |
| | | TVPPTGTEPGTVIIETPESYVTTTQPWTGTYETTYTVPPT |
| | | GTEPGTVIIETPESYVTTTQPWTGTYETTYTVPPTGTEPG |
| | | TVIIETPEIIDCEAVCCGPFLTAFSFRKREECQCENICCP |
| | | GDTNCETYVTTTQPWTGTYETTYTVPPTGTEPGTVIIETP |
| | | ESYVTTTQPWTGTYETTYTVPPTGTEPGTVIIETPESYVT |
| | | TTQPWTGTYETTYTVPPTGTEPGTVIIETPEIINCEAVCC |
| | | GPFLTAFSFRKREECQCENICCPGDTNCETYVTTTQPWTG |
| | | TYETTYTVPPTGTEPGTVIIETPESYVTTTQPWTGTYETT |
| | | YTVPSTGTEPGTVIIETPESYVTTTQPWTGTYETTFTVPP |
| | | TGTEPGTVVIETPESYVTTTQPWTGTYETTYSVPPSGTEP |
| | | GTVVIETPESYVTTTQPWTGTYETTYSVPPSGTEPGTVVI |
| | | ETPEASTARTKFTTVTSSWTGVFTTTKTLPASGTEPATIV |
| | | IQTPTGYFNTSSLVSTRTKTNVDTVTRVIPCPICTAPKTI |
| | | TVVPEEPNESVSVIISQPQSSSTDTTLSKPDSVRVISQPE |
| | | TASQMDTSLSKTDSAVISTETAGNNIIPLAGSHSYNTIVT |
| | | TVTDSPQVAQSTTATSSSNVHLTISTQTTTPSLVYSSSLS |
| | | TVHQVSPSNGGFRSSITVHPLLSVIGAIFGALFM |
| EndoH-Flo11 fusion | SEQ ID NO: 13 | APAPVKQGPTSVAYVEVNNNSMLNVGKYTLADGGGNAFDV |
| (partial ORF, without | | AVIFAANINYDTGTKTAYLHFNENVQRVLDNAVTQIRPLQ |
| signal peptide that is | | QQGIKVLLSVLGNHQGAGFANFPSQQAASAFAKQLSDAVA |
| cleaved off post- | | KYGLDGVDFDDEYAEYGNNGTAQPNDSSFVHLVTALRANM |
| translationally) | | PDKIISLYNIGPAASRLSYGGVDVSDKFDYAWNPYYGTWQ |
| | | VPGIALPKAQLSPAAVEIGRTSRSTVADLARRTVDEGYGV |
| | | YLTYNLDGGDRTADVSAFTRELYGSEAVRTPGSSGSSGSS |
| | | GSSGSSGSSGSSGSSEAAAREAAAREAAAREAAARGGGGS |
| | | GGGGSGGGGSSSGKTCPTSEVSPACYANQWETTFPPSDIK |
| | | ITGATWVQDNIYDVTLSYEAESLELENLTELKIIGLNSPT |
| | | GGTKLVWSLNSKVYDIDNPAKWTTTLRVYTKSSADDCYVE |
| | | MYPFQIQVDWCEAGASTDGCSAWKWPKSYDYDIGCDNMQD |
| | | GVSRKHHPVYKWPKKCSSNCGVEPTTSDEPEEPTTSEEPE |
| | | EPTTSEEPEEPTSSDEEPTTSEEPEEPTTSDEPEEPTTSE |
| | | EPEEPTTSEEPEEPTTSEEPTTSEEPEEPTSSDEEPTTSD |
| | | EPEEPTTSDEPEEPTTSEEPTTSEEPEEPTTSSEEPTPSE |
| | | EPEGPTCPTSEVSPACYADQWETTFPPSDIKITGATWVED |
| | | NIYDVTLSYEAESLELENLTELKIIGLNSPTGGTKVVWSL |
| | | NSGIYDIDNPAKWTTTLRVYTKSSADDCYVEMYPFQIQVD |
| | | WCEAGASTDGCSAWKWPKSYDYDIGCDNMQDGVSRKHHPV |
| | | YKWPKKCSSDCGVEPTTSDEPEEPTTSEEPVEPTSSDEEP |
| | | TTSEEPTTSEEPEEPTTSDEPEEPTTSEEPEEPTTSEEPE |
| | | EPTTSEEPTTSEEPEEPTSSDEEPTTSDEPEEPTTSEEPE |
| | | EPTTSEEPEEPTTSEEPEEPTTSDEPEEPTTSEEPEEPTT |
| | | SEEPEEPTSSDEEPTTSEEPEEPTTSEEPEEPTTSEEPEE |
| | | PTTSEEPEEPTSSDEEPTTSEEPEEPTTSDEPEEPTTSEE |
| | | PEEPTTSEEPEEPTSSDEEPTTSEEPEEPTTSDEPEEPTT |
| | | SEEPEEPTTSEEPEEPTTSEEPEEPTTSEEPEEPTSSDEE |
| | | PTTSEEPEEPTTSDEPEEPTTSEEPEEPTTSEEPEEPTTS |
| | | EEPEEPTTSDEEPGTTEEPLVPTTKTETDVSTTLLTVTDC |
| | | GTKTCTKSLVITGVTKETVTTHGKTTVITTYCPLPTETVT |
| | | PTPVTVTSTIYADESVTKTTVYTTGAVEKTVTVGGSSTVV |
| | | VVHTPLTTAVVQSQSTDEIKTVVTARPSTTTIVRDVCYNS |
| | | VCSVATIVTGVTEKTITFSTGSITVVPTYVPLVESEEHQR |
| | | TASTSETRATSVVVPTVVGQSSSASATSSIFPSVTIHEGV |
| | | ANTVKNSMISGAVALLFNALFL |
| EndoH-Flo11 fusion | SEQ ID NO: 14 | MVSLRSIFTSSILAAGLTRAHGAPAPVKQGPTSVAYVEVN |
| (full ORF, including | | NNSMLNVGKYTLADGGGNAFDVAVIFAANINYDTGTKTAY |
| signal peptide that is | | LHFNENVQRVLDNAVTQIRPLQQQGIKVLLSVLGNHQGAG |
| cleaved off post- | | FANFPSQQAASAFAKQLSDAVAKYGLDGVDFDDEYAEYGN |
| translationally) | | NGTAQPNDSSFVHLVTALRANMPDKIISLYNIGPAASRLS |
| | | YGGVDVSDKFDYAWNPYYGTWQVPGIALPKAQLSPAAVEI |
| | | GRTSRSTVADLARRTVDEGYGVYLTYNLDGGDRTADVSAF |
| | | TRELYGSEAVRTPGSSGSSGSSGSSGSSGSSGSSGSSEAA |
| | | AREAAAREAAAREAAARGGGGSGGGGSGGGGSSSGKTCPT |
| | | SEVSPACYANQWETTFPPSDIKITGATWVQDNIYDVTLSY |
| | | EAESLELENLTELKIIGLNSPTGGTKLVWSLNSKVYDIDN |
| | | PAKWTTTLRVYTKSSADDCYVEMYPFQIQVDWCEAGASTD |
| | | GCSAWKWPKSYDYDIGCDNMQDGVSRKHHPVYKWPKKCSS |
| | | NCGVEPTTSDEPEEPTTSEEPEEPTTSEEPEEPTSSDEEP |
| | | TTSEEPEEPTTSDEPEEPTTSEEPEEPTTSEEPEEPTTSE |
| | | EPTTSEEPEEPTSSDEEPTTSDEPEEPTTSDEPEEPTTSE |
| | | EPTTSEEPEEPTTSSEEPTPSEEPEGPTCPTSEVSPACYA |
| | | DQWETTFPPSDIKITGATWVEDNIYDVTLSYEAESLELEN |
| | | LTELKIIGLNSPTGGTKVVWSLNSGIYDIDNPAKWTTTLR |
| | | VYTKSSADDCYVEMYPFQIQVDWCEAGASTDGCSAWKWPK |
| | | SYDYDIGCDNMQDGVSRKHHPVYKWPKKCSSDCGVEPTTS |
| | | DEPEEPTTSEEPVEPTSSDEEPTTSEEPTTSEEPEEPTTS |
| | | DEPEEPTTSEEPEEPTTSEEPEEPTTSEEPTTSEEPEEPT |
| | | SSDEEPTTSDEPEEPTTSEEPEEPTTSEEPEEPTTSEEPE |
| | | EPTTSDEPEEPTTSEEPEEPTTSEEPEEPTSSDEEPTTSE |
| | | EPEEPTTSEEPEEPTTSEEPEEPTTSEEPEEPTSSDEEPT |
| | | TSEEPEEPTTSDEPEEPTTSEEPEEPTTSEEPEEPTSSDE |
| | | EPTTSEEPEEPTTSDEPEEPTTSEEPEEPTTSEEPEEPTT |
| | | SEEPEEPTTSEEPEEPTSSDEEPTTSEEPEEPTTSDEPEE |
| | | PTTSEEPEEPTTSEEPEEPTTSEEPEEPTTSDEEPGTTEE |
| | | PLVPTTKTETDVSTTLLTVTDCGTKTCTKSLVITGVTKET |
| | | VTTHGKTTVITTYCPLPTETVTPTPVTVTSTIYADESVTK |
| | | TTVYTTGAVEKTVTVGGSSTVVVVHTPLTTAVVQSQSTDE |
| | | IKTVVTARPSTTTIVRDVCYNSVCSVATIVTGVTEKTITF |
| | | STGSITVVPTYVPLVESEEHQRTASTSETRATSVVVPTVV |
| | | GQSSSASATSSIFPSVTIHEGVANTVKNSMISGAVALLFN |
| | | ALFL |
| FLO5 Saccharomyces | SEQ ID NO: 20 | MTIAHHCIFLVILAFLALINVASGATEACLPAGQRKSGMN |
| cerevisiae | | INFYQYSLKDSSTYSNAAYMAYGYASKTKLGSVGGQTDIS |
| | | IDYNIPCVSSSGTFPCPQEDSYGNWGCKGMGACSNSQGIA |
| | | YWSTDLFGFYTTPTNVTLEMTGYFLPPQTGSYTFSFATVD |
| | | DSAILSVGGSIAFECCAQEQPPITSTNFTINGIKPWDGSL |
| | | PDNITGTVYMYAGYYYPLKVVYSNAVSWGTLPISVELPDG |
| | | TTVSDNFEGYVYSFDDDLSQSNCTIPDPSIHTTSTITTTT |
| | | EPWTGTFTSTSTEMTTITDTNGQLTDETVIVIRTPTTAST |
| | | ITTTTEPWTGTFTSTSTEMTTVTGTNGQPTDETVIVIRTP |
| | | TSEGLITTTTEPWTGTFTSTSTEMTTVTGTNGQPTDETVI |
| | | VIRTPTSEGLITTTTEPWTGTFTSTSTEVTTITGTNGQPT |
| | | DETVIVIRTPTSEGLITTTTEPWTGTFTSTSTEMTTVTGT |
| | | NGQPTDETVIVIRTPTSEGLISTTTEPWTGTFTSTSTEVT |
| | | TITGTNGQPTDETVIVIRTPTSEGLITTTTEPWTGTFTST |
| | | STEMTTVTGTNGQPTDETVIVIRTPTSEGLITRTTEPWTG |
| | | TFTSTSTEVTTITGTNGQPTDETVIVIRTPTTAISSSLSS |
| | | SSGQITSSITSSRPIITPFYPSNGTSVISSSVISSSVTSS |
| | | LVTSSSFISSSVISSSTTTSTSIFSESSTSSVIPTSSSTS |
| | | GSSESKTSSASSSSSSSSISSESPKSPTNSSSSLPPVTSA |
| | | TTGQETASSLPPATTTKTSEQTTLVTVTSCESHVCTESIS |
| | | SAIVSTATVTVSGVTTEYTTWCPISTTETTKQTKGTTEQT |
| | | KGTTEQTTETTKQTTVVTISSCESDICSKTASPAIVSTST |
| | | ATINGVTTEYTTWCPISTTESKQQTTLVTVTSCESGVCSE |
| | | TTSPAIVSTATATVNDVVTVYPTWRPQTTNEQSVSSKMNS |
| | | ATSETTTNTGAAETKTAVTSSLSRFNHAETQTASATDVIG |
| | | HSSSVVSVSETGNTMSLTSSGLSTMSQQPRSTPASSMVGS |
| | | STASLEISTYAGSANSLLAGSGLSVFIASLLLAII |
| N-terminal addition | SEQ ID NO: 21 | EAEA |
| EAEA | | |
| GGGS linker | SEQ ID NO: 22 | GGGGS |
| GSS linker | SEQ ID NO: 23 | GSS |
| A rigid linker that | SEQ ID NO: 24 | EAAAREAAAREAAAREAAAR |
| forms 4 turns of an | | |
| alpha helix | | |
| Full linker | SEQ ID NO: 25 | GSSGSSGSSGSSGSSGSSGSSGSSEAAAREAAAREAAARE |
| | | AAARGGGGSGGGGSGGGGS |
| AOX1 promoter | SEQ ID NO: 26 | GATCTAACATCCAAAGACGAAAGGTTGAATGAAACCTTTT |
| | | TGCCATCCGACATCCACAGGTCCATTCTCACACATAAGTG |
| | | CCAAACGCAACAGGAGGGGATACACTAGCAGCAGACCGTT |
| | | GCAAACGCAGGACCTCCACTCCTCTTCTCCTCAACACCCA |
| | | CTTTTGCCATCGAAAAACCAGCCCAGTTATTGGGCTTGAT |
| | | TGGAGCTCGCTCATTCCAATTCCTTCTATTAGGCTACTAA |
| | | CACCATGACTTTATTAGCCTGTCTATCCTGGCCCCCCTGG |
| | | CGAGGTTCATGTTTGTTTATTTCCGAATGCAACAAGCTCC |
| | | GCATTACACCCGAACATCACTCCAGATGAGGGCTTTCTGA |
| | | GTGTGGGGTCAAATAGTTTCATGTTCCCCAAATGGCCCAA |
| | | AACTGACAGTTTAAACGCTGTCTTGGAACCTAATATGACA |
| | | AAAGCGTGATCTCATCCAAGATGAACTAAGTTTGGTTCGT |
| | | TGAAATGCTAACGGCCAGTTGGTCAAAAAGAAACTTCCAA |
| | | AAGTCGGCATACCGTTTGTCTTGTTTGGTATTGATTGACG |
| | | AATGCTCAAAAATAATCTCATTAATGCTTAGCGCAGTCTC |
| | | TCTATCGCTTCTGAACCCCGGTGCACCTGTGCCGAAACGC |
| | | AAATGGGGAAACACCCGCTTTTTGGATGATTATGCATTGT |
| | | CTCCACATTGTATGCTTCCAAGATTCTGGTGGGAATACTG |
| | | CTGATAGCCTAACGTTCATGATCAAAATTTAACTGTTCTA |
| | | ACCCCTACTTGACAGCAATATATAAACAGAAGGAAGCTGC |
| | | CCTGTCTTAAACCTTTTTTTTTATCATCATTATTAGCTTA |
| | | CTTTCATAATTGCGACTGGTTCCAATTGACAAGCTTTTGA |
| | | TTTTAACGACTTTTAACGACAACTTGAGAAGATCAAAAAA |
| | | CAACTAATTATTGGATCCCGA |
| DAK2 promoter | SEQ ID NO: 27 | AAATAAGCATGTTTGTTTCAGATCAAAGATTAGCGTTTCA |
| | | AAGTTGTGGAAAAGTGACCATGCAACAATATGCAACACAT |
| | | TCGGATTATCTGATAAGTTTCAAAGCTACTAAGTAAGCCC |
| | | GTTTCAAGTCTCCAGACCGACATCTGCCATCCAGTGATTT |
| | | TCTTAGTCCTGAAAAATACGATGTGTAAACATAAACCACA |
| | | AAGATCGGCCTCCGAGGTTGAACCCTTACGAAAGAGACAT |
| | | CTGGTAGCGCCAATGCCAAAAAAAAATCACACCAGAAGGA |
| | | CAATTCCCTTCCCCCCCAGCCCATTAAAGCTTACCATTTC |
| | | CTATTCCAATACGTTCCATAGAGGGCATCGCTCGGCTCAT |
| | | TTTCGCGTGGGTCATACTAGAGCGGCTAGCTAGTCGGCTG |
| | | TTTGAGCTCTCTAATCGAGGGGTAAGGATGTCTAATATGT |
| | | CATAATGGCTCACTATATAAAGAACCCGCTTGCTCAACCT |
| | | TCGACTCCTTTCCCGATCCTTTGCTTGTTGCTTCTTCTTT |
| | | TATAACAGGAAACAAAGGAATTTATACACTTTAAGAATT |
| PEX11 promoter | SEQ ID NO: 28 | CTTCCCCATTTCACTGACAGTTTGTAGAAATAGGGCAACA |
| | | ATTGATGCAAATCGATTTTCAACGCATTGGTTTTGATAGC |
| | | ATTGATGATCTTGGAGCTGTAAAAGTCCGGCTGGATAAGC |
| | | TCAATGAAATAGGTTGGTTGATCTGGATCTTCTTTTGGGT |
| | | CATTTTGTTCGCTCTGTATTTCACAAATTGCCAGAATCTC |
| | | TGCCAACCACAGTGGTAGGTCCAACTTGGTGTTCTGAATC |
| | | ACAGGCTTCCCCGGGTTGTTCTCTAAATAACCGAGGCCCG |
| | | GCACAGAAATCGTAAACCGACACGGTATCTTTTGTCCGTC |
| | | CGCCAGTATCTCATCAAGGTCGTAGTAGCCCATGATGAGT |
| | | ATCAAAGGGGATTTGGTTATGCGATGCAACGAGAGATTGT |
| | | TTATCCCAGATGCTGATGTAAAAACCTTAACCAGCGTGAC |
| | | AGTAGAAATAAGACACGTTAAAATTACCCGCGCTTCCCTA |
| | | ACAATTGGCTCTGCCTTTCGGCAAGTTTCTAACTGCCCTC |
| | | CCCTCTCACATGCACCACGAACTTACCGTTCGCTCCTAGC |
| | | AGAACCACCCCAAAGTTTAATCAGGACCGCATTTTAGCCT |
| | | ATTGCTGTAGAACCCCACAACATAACCTGGTCCAGAGCCA |
| | | GCCCTTTATATATGGTAAATCCCGTTTGAACTTCGAAGTG |
| | | GAATCGGAATTTTTACATCAAAGAAACTGATACTGAAACT |
| | | TTTGGCTTCGACTTGGACTTTCTCTTAATC |
| FLD1 promoter | SEQ ID NO: 29 | AAATCAGCCATTAATCTCACCTCAGTTTTTGAATCAGTAG |
| | | AATTTTCAATGAAACAAACGGTTGGTATATTATTTGATAG |
| | | GGTAGCCAAATTTCCAAAAATGAACTTTTCATCAGGTAAT |
| | | ATCTTGAATACCGTAATGTAGTGACTATTGGAAGAAACTG |
| | | CTATCAAATTATATTTCGGATAGAAATCCAAACCCCAGAC |
| | | TGATCTCTTGAGTCTCAACTCTAAGTCAGCCGCGACTCTA |
| | | ATTATCTGTGGATTAGGAGTTAGTGTGGACAAAGCATCAG |
| | | TATAGTATAACTTTACGGTTCCATTATCAGACGCTATTGC |
| | | AAGAACTTCCTTTCCATTGATCTCTCCAATTCGACAGTAA |
| | | TTGATATCATAAGGTAGGTCTGGAAACACACTGGCGCTTG |
| | | TATCCCATTCTGCAGGAATTTCTGGAACGGTGGTAATGGT |
| | | AGTTATCCAACGGAGTTGGGGTAGTTGGTATATCTGGATA |
| | | TGCCGCCTATAGGATAAAAACAGGAGAGAGTGAACCTTGC |
| | | TTACGGCTACTAGATTGTTCTTGTACTCGGAATTGTCGTT |
| | | ATCGGAAACTAGACTAATCTCATCTGTGTGTTGCAGTACT |
| | | ATTGAGTCGTTGTAGTATCTACCAGGAGGGCATTCCATGA |
| | | ACTAGTGAGACAAATGAGTTGGATTTTCTCAATAGACATA |
| | | TGCAAGAATGCTACACAACGGATGTCGCACTCTTTTTCTT |
| | | AGTTGATAATATCATCCAATCAGAAGACACGGGCTAGAAG |
| | | GACTTGCTCCCGAAGGATAATCCACTGCTACTATCTCCCT |
| | | TCCTCACATATAGTCTTGCAGGGCTCATGCCCCTTTCTCC |
| | | TTCGAACTGCCCGATGAGGAAGTCTTTAGCCTATCAAGGA |
| | | ATTCGGGACCATCATCAATTTTTAGAGCCTTACCTGATCG |
| | | CAATCAGGATTTCACTACTCATATAAATACATCACTCAAA |
| | | CTCCAACTTTGCTTGTTCATACAATTCTTGATATTCACAG |
| | | GATC |
| FGH1 promoter | SEQ ID NO: 30 | GTGAATTTGTCACGGAATTGACCAAGAGGTCAGACGATCC |
| | | TGTATCCCATTGAGCCGTTATGCTTTGTGGGGGAAACCCT |
| | | ATTTCTATCGTACTAAGAAAACCAATGGTGAACTCATATT |
| | | CGGTATCAATGGCGACGATTCCAGCATAGCCTGTAGACAG |
| | | TAACAACACTAGGGCAACAGCAACTAACATATCTTCATTG |
| | | ATGAAACGTTGTGATCGGTGTGACTTTTATAGTAAAAGCT |
| | | ACAACTGTTTGAAATACCAAGATATCATTGTGAATGGCTC |
| | | AAAAGGGTAATACATCTGAAAAACCTGAAGTGTGGAAAAT |
| | | TCCGATGGAGCCAACTCATGATAACGCAGAAGTCCCATTT |
| | | TGCCATCTTCTCTTGGTATGAAACGGTAGAAAATGATCCG |
| | | AGTATGCCAATTGATACTCTTGATTCATGCCCTATAGTTT |
| | | GCGTAGGGTTTAATTGATCTCCTGGTCTATCGATCTGGGA |
| | | CGCAATGTAGACCCCATTAGTGGAAACACTGAAAGGGATC |
| | | CAACACTCTAGGCGGACCCGCTCACAGTCATTTCAGGACA |
| | | ATCACCACAGGAATCAACTACTTCTCCCAGTCTTCCTTGC |
| | | GTGAAGCTTCAAGCCTACAACATAACACTTCTTACTTAAT |
| | | CTTTGATTCTCGAATTGTTTACCCAATCTTGACAACTTAG |
| | | CCTAAGCAATACTCTGGGGTTATATATAGCAATTGCTCTT |
| | | CCTCGCTGTAGCGTTCATTCCATCTTTCTAGAATTCGT |
| DAS2 promoter | SEQ ID NO: 31 | CCTGTTGATAAGACGCATTCTAGAGTTGTTTCATGAAAGG |
| | | GTTACGGGTGTTGATTGGTTTGAGATATGCCAGAGGACAG |
| | | ATCAATCTGTGGTTTGCTAAACTGGAAGTCTGGTAAGGAC |
| | | TCTAGCAAGTCCGTTACTCAAAAAGTCATACCAAGTAAGA |
| | | TTACGTAACACCTGGGCATGACTTTCTAAGTTAGCAAGTC |
| | | ACCAAGAGGGTCCTATTTAACGTTTGGCGGTATCTGAAAC |
| | | ACAAGACTTGCCTATCCCATAGTACATCATATTACCTGTC |
| | | AAGCTATGCTACCCCACAGAAATACCCCAAAAGTTGAAGT |
| | | GAAAAAATGAAAATTACTGGTAACTTCACCCCATAACAAA |
| | | CTTAATAATTTCTGTAGCCAATGAAAGTAAACCCCATTCA |
| | | ATGTTCCGAGATTTAGTATACTTGCCCCTATAAGAAACGA |
| | | AGGATTTCAGCTTCCTTACCCCATGAACAGAAATCTTCCA |
| | | TTTACCCCCCACTGGAGAGATCCGCCCAAACGAACAGATA |
| | | ATAGAAAAAAGAAATTCGGACAAATAGAACACTTTCTCAG |
| | | CCAATTAAAGTCATTCCATGCACTCCCTTTAGCTGCCGTT |
| | | CCATCCCTTTGTTGAGCAACACCATCGTTAGCCAGTACGA |
| | | AAGAGGAAACTTAACCGATACCTTGGAGAAATCTAAGGCG |
| | | CGAATGAGTTTAGCCTAGATATCCTTAGTGAAGGGTTGTT |
| | | CCGATACTTCTCCACATTCAGTCATAGATGGGCAGCTTTG |
| | | TTATCATGAAGAGACGGAAACGGGCATTAAGGGTTAACCG |
| | | CCAAATTATATAAAGACAACATGTCCCCAGTTTAAAGTTT |
| | | TTCTTTCCTATTCTTGTATCCTGAGTGACCGTTGTGTTTA |
| | | ATATAACAAGTTCGTTTTAACTTAAGACCAAAACCAGTTA |
| | | CAACAAATTATAACCCCTCTAAACACTAAAGTTCACTCTT |
| | | ATCAAACTATCAAACATCAAAAGAATTCGCG |
| CAT1 promoter | SEQ ID NO: 32 | TAATCGAACTCCGAATGCGGTTCTCCTGTAACCTTAATTG |
| | | TAGCATAGATCACTTAAATAAACTCATGGCCTGACATCTG |
| | | TACACGTTCTTATTGGTCTTTTAGCAATCTTGAAGTCTTT |
| | | CTATTGTTCCGGTCGGCATTACCTAATAAATTCGAATCGA |
| | | GATTGCTAGTACCTGATATCATATGAAGTAATCATCACAT |
| | | GCAAGTTCCATGATACCCTCTACTAATGGAATTGAACAAA |
| | | GTTTAAGCTTCTCGCACGAGACCGAATCCATACTATGCAC |
| | | CCCTCAAAGTTGGGATTAGTCAGGAAAGCTGAGCAATTAA |
| | | CTTCCCTCGATTGGCCTGGACTTTTCGCTTAGCCTGCCGC |
| | | AATCGGTAAGTTTCATTATCCCAGCGGGGTGATAGCCTCT |
| | | GTTGCTCATCAGGCCAAAATCATATATAAGCTGTAGACCC |
| | | AGCACTTCAATTACTTGAAATTCACCATAACACTTGCTCT |
| | | AGTCAAGACTTACAATTAAA |
| MDH3 promoter | SEQ ID NO: 33 | TAGCTTGGGTAGGACTTGACAAGTACGGCTTCCGTGGTCA |
| | | TACCAAACGCCTTTGTTACCGTTGGCTATACCTAATGACC |
| | | AAGGCATTTGTGGATTATAACGGTATCGTAGTTGAAAAAT |
| | | ATGACGTAACCACTGGTACTAGCCCCCACAAGGTTGATGC |
| | | TGAATACGGGAATCAAGGTGCCGATTTTAAAGGAGTAGCC |
| | | ACTGAAGGGTTTGGCTGGGTCAATGCCTCTTTTATTTTGG |
| | | GATTAACCTACTTAGATGTCCAAGGCATCCGTGCGATAGG |
| | | CGCCGTTACGTCCCCTGATGTATTTTTCAGGAAGCTCAAA |
| | | CCTTGGGAACGCGCAAGTTATGGCCTAAGGCCATGTAACG |
| | | AGATAGTCAAGTCAAACTAGAAGTATACGGTTTCCCCGCA |
| | | GAAATAGCAGAAATAGGCGACAAATACATACAACATTTTC |
| | | ATTGTGATAGGGGGCGGCGGTTCCTAGGAGGGACAACCCC |
| | | CAGAAACCTTGTAGACTACGTTTTCACGACGATGGGTTAT |
| | | TACTGTAAAGGAAGAATATACTACCCACCAGTTGAATGTT |
| | | TGAACGGATCAAAGGTCGAAGGGAGTACACGGCCCAACCA |
| | | ACGTAGCTACCGGAGAAAGCAAGACTTTCCCAAACCAAAT |
| | | AGCTCCGGGTTTCTTCTCCGGCAACCCGTCAGTTTTTGTG |
| | | TGGCCGGACAAAAATTCGCACCCTCAGTCTAATTGAAAGG |
| | | TCGGGCTCCGAGCTCTAGGCGTTTGCGCATGTAATATTGC |
| | | ATCCCCTCCCATAGATAATACTGCGCGAACACAGGGTGCA |
| | | AATTATGATGACCACACATGCCAGTGACCAAAACAGTTTT |
| | | TTAGTCTTTAAAAACCCTCGGAACTTCTGAGTATATAAAG |
| | | GCTTCTCATTTCCTACAAGCAAACAAAGAAGAAACTTCCA |
| | | CTTTCTAACTTTTTATCTATAGACTTTAGAGTTACAACCA |
| | | ACGAACAATAACAAA |
| HAC1 promoter | SEQ ID NO: 34 | TGAAGCTTATCTGCTGAGCAAGTTGTTTGACCAAACTTGA |
| | | GTCAACAGTGGTTAACTATATCCTCTATTATTTTAGATGG |
| | | GAGCACATCAAGTGTACGGGAACAATGCAATCGACAACCT |
| | | GTAGCCTGACATACATAGCCATCTTGAATTGACAAAACTT |
| | | AGAATGTCTTGAATGTGATAGATATGAGTTCCCAAAAATC |
| | | TCTTTTACGATTTCCCAGTTGCGGTGTACTATTACACAGA |
| | | GGATATCATAGCAGACTTACAATCCTCAGGCATAAAACGA |
| | | GCTTTCTTATCAAAGTGTATTCAAATGGACCATTTGATTG |
| | | CACCAAGGCATTAGCCCCAAACCATACCACACAGTAACTT |
| | | GATATTCTCAGCATGCATGGAAATTCCACTCATAACGCGC |
| | | TATTCACCGCGAATACTTATCTATGAAACTGGGTTCTTTA |
| | | GTATTCTTTGCCAAATTTCACCGATTAGAAATTATTAGGT |
| | | AATATAATTTCTTTGGGGAACCCCTTCCCGTTACGCCCGC |
| | | TGCGGCTTTGTGGTTCTTTTCCAGTCTTGAGCAAATTACA |
| | | TCTGGTCTAGACAGTTCTTCCGTGCCCCAGTATGCGAGCG |
| | | CAAACTTTCAATCAAACCTCGTAGCAAATTGGTACTTGAA |
| | | CTTCGTATTTAACCGCTATTAAATGTACTGACTCTTACAT |
| | | TATGAAAAATTTTGATAAAGATTTTATATTTCATCTCAGT |
| | | TAATCTCCTAATAATAATAGTCTGCATAACTCAAACGGTA |
| | | CTTCCTTTTCGGAACGCGAAGAGTAGTCTCTATGTCATTC |
| | | TCACACTATCCGCAGCGCAATAGAGAACGAGCATGTTACC |
| | | CGACTCATCCCTTGTCGATTCGGAAACGATTTATAAATAC |
| | | AATTAGATCGCCACCGATCTTCTTTTGTCAATATTATAAA |
| | | AATAGTACAGATTTTCCTTAGTCGAATCAGATCGCAGAAA |
| BiP promoter | SEQ ID NO: 35 | AGATCTGAGGGTGTATACGATGTATCGTGCCGAACACATG |
| | | CACTTGACGGCACAGCAAATGGTATTCAAGAAGACCACTT |
| | | TAGAATGGGAGTTAATAGGGATGGTTTCATGGAGGTTAAA |
| | | ACACTTCAAGGAGGCATCTGAAGCATTCAAGTATGCACTA |
| | | GGTCTGAGGTTTTCGGTCAAGGCATGCAAGAAATTAATTG |
| | | TATTCTATCTGAACGAACGCTCCAGAATGAACCAGCCAGA |
| | | AACCTCAATTGCCCTCAACAACTTAAATCAATCCACATTA |
| | | TCCATCCAAGAGATTCTCAAGTATCGTTCGTTCCTCGATA |
| | | TCAACCTAATTTCAAACTTGGTCAAACTAGGAGTTTGGAA |
| | | TCACCGCTGGTATGCTGAGTTTTCTCCAAAACTCATAGAA |
| | | AGCCTTGCGGTTGTTGTGGAGAACGGAGGGCTTATCAAGG |
| | | TAGAAAACGAGGTTAAGGCTACCTATTTCGATTCACAAGA |
| | | TGGAGTTTACGACTTGATGAACGAGGTATTCAAGTTCATG |
| | | AAGCATTACGATTATCCTGGGACTGACAACTAAGAGCTCC |
| | | TAGTGAAGACTTGAGATGGACATGATAAACAATTATAGTG |
| | | AAAATAGAAACCATAATACAATATTCTAATAGAGGAACCG |
| | | TTTACCTGTGGTTCCTATTGTGGCCTACTGTTACTAGCTA |
| | | GTGTAATACACCCTTGCCTCAGCTTTGCAAGTTGACAACT |
| | | CAGCCAAATGATCTTTGAATGCGCGAAACCTCAAGGTCCA |
| | | TCGAATTTTCTCGAATTTTCAGTGTTTTCATACAGCGTGT |
| | | CATCTTCTTTCGCGTACTTATTAAAATCGTACCCAGATCC |
| | | CTTCTTCTTCCTTAATTTCAATTCCAACACTCAAGA |
| RAD30 promoter | SEQ ID NO: 36 | AGATCTTGCAAAATACCTTTCCAGCTTTCCAGCTTCCTAG |
| | | CACTCATCTTGAAGATATCAAATATTCTCCATTCAAACCA |
| | | ACATCAAAAAATAGAATAATTATAATCAGTTTGAAGAGCA |
| | | AGAGTAATTTTAAAGGAAACACATTCATGGTCAGCTAGAA |
| | | GGTTGACTGAAGAGTCGCAAGATATCTGAGAATAAAAAAG |
| | | AGCATAGCTAACAAGATGAGTAAACACGGCAAACAGATTT |
| | | AGGAACAGGTGAAGGGTTTCTGGCTCTTCAATGTATATCC |
| | | TGCTAGCCACCCATTCAGAAATAACACAAAGTAGGACCCT |
| | | ACTGAAAAATAAATTTAATACATCTTCATCCTCTCATTAA |
| | | ACCACCGACCACTCAAACCATACCAGCCTTGTCCAATTCC |
| | | ATGCATCGTGCTATCCGTCAGAATTTTCAGTGTTAATCGA |
| | | ATCGGTCATTATAGCTCCGTCTGGGGCGACAACTTGTCAT |
| | | CACAGAATAGCACAATTATGCGTTGGAATCGTCAAAAAAT |
| | | CACCTCCAGGTCTGTATACATACAGAACTGGTTGTAACGA |
| | | CAACCTTGTTTGATTGAGGTGACTGGAAGGTGGAAAGAAA |
| | | GGGAGGAAATAAATATTGCAAGGAAAGAAAAAAAAATTGT |
| | | TCACAGTCACCTCTTCACCTTCGCGATTTCATGTTTCTTT |
| | | CATGTGCTAACTGATCCCAGGGCTTCTCCAGCGCCCTTAT |
| | | CTGTTAG |
| RVS161-2 promoter | SEQ ID NO: 37 | CTGCCCATCTATGACTGAATGTGGAGAAGTATCGGAACAA |
| | | CCCTTCACTAAGGATATCTAGGCTAAACTCATTCGCGCCT |
| | | TAGATTTCTCCAAGGTATCGGTTAAGTTTCCTCTTTCGTA |
| | | CTGGCTAACGATGGTGTTGCTCAACAAAGGGATGGAACGG |
| | | CAGCTAAAGGGAGTGCATGGAATGACTTTAATTGGCTGAG |
| | | AAAGTGTTCTATTTGTCCGAATTTCTTTTTTCTATTATCT |
| | | GTTCGTTTGGGCGGATCTCTCCAGTGGGGGGTAAATGGAA |
| | | GATTTCTGTTCATGGGGTAAGGAAGCTGAAATCCTTCGTT |
| | | TCTTATAGGGGCAAGTATACTAAATCTCGGAACATTGAAT |
| | | GGGGTTTACTTTCATTGGCTACAGAAATTATTAAGTTTGT |
| | | TATGGGGTGAAGTTACCAGTAATTTTCATTTTTTCACTTC |
| | | AACTTTTGGGGTATTTCTGTGGGGTAGCATAGCTTGACAG |
| | | GTAATATGATGTACTATGGGATAGGCAAGTCTTGTGTTTC |
| | | AGATACCGCCAAACGTTAAATAGGACCCTCTTGGTGACTT |
| | | GCTAACTTAGAAAGTCATGCCCAGGTGTTACGTAATCTTA |
| | | CTTGGTATGACTTTTTGAGTAACGGACTTGCTAGAGTCCT |
| | | TACCAGACTTCCAGTTTAGCAAACCACAGATTGATCTGTC |
| | | CTCTGGCATATCTCAAACCAATCAACACCCGTAACCCTTT |
| | | CATGAAACAACTCTAGAATGCGTCTTATCAACAGGATTGC |
| | | CCAAAACAGTAATTGGGGCGGTGGAATCTACATGGGAGTT |
| | | CCATCGTTGTCTCGGTTTTTCTCCCTATAAGCTACTCTGG |
| | | AGACGAAGTAACTAACACCCTCAAATATCATT |
| MPP10 promoter | SEQ ID NO: 38 | TCTGAATCCGACCTCCTCTAATCTACCACTGAAGAGAAGC |
| | | AGTGTATTGTTCGTCTACGTAAATTTGAATGTGTAAATGG |
| | | CAAACATGGCTTCGGGGATGATTTGGCATATATATTATTG |
| | | TAGCATCGTCTGTGGCTCTATGAGTTGTGTGGCGGATGAT |
| | | GAAAAGTTTCGTGCTGATCCCACAATGCGGCATTTACCAA |
| | | ATGGGGAAAGACCAGATTTCTTCGCTGCGCCAGCTAGGGA |
| | | CAGCATAATGTTCCAAGAAGAAGCGATTACAGGTGGATTA |
| | | CAAAGCGTTCGTCTGCAGTTGATGTTCTACGTGATGGGTA |
| | | TGAGTTGTAGTGCTACGCTCCATGAATACTTCTAATTTGT |
| | | CGTTGACAATCCATGAATAATTTAAGTTTGCTTCCCAAGA |
| | | GTCTATTGCGAAGGGTGAGCCGAATCTCTTGGCGTATGCA |
| | | CCCGACTCGTCGGCTTTTGTGCGTTCCTTGCAAAGCTCGG |
| | | TAGCAATCCGTTGGTGGGAGAAATTTGTCTCACGAATTTC |
| | | AGTTGGGAGTAGCTGTTCCTGGTAGCAAGTTCGAGGGGAT |
| | | CTGTGCTCATAAAACGTGCTCACGCCAAAAATATTCTTAC |
| | | AAAATCTTCGCGGGGTGTTTGTCTTACATAATCGATTGGA |
| | | TATTTTCTTCAAATTTTTTTTTCTTACTGAAGTCCCCTAT |
| | | AGAG |
| THP3 promoter | SEQ ID NO: 39 | TCTTGCCAGTTGTCTCCTAAGATGTCATCGGAGTAGGCTC |
| | | GGCTAAAGAGTAGTAATGCATCAAGACCAACCAAAACACC |
| | | TTCCACGAGTTCAGATGAACC |
| | | TTTTAATAACTTCAGGTCACTTTGATGCCGGCACAACTGG |
| | | GCGAGTTTCGTATAGTTAACTCTGATCTTGCACTCCAGAA |
| | | CGGGAATAGGATTGACTTTTTGCTTCCGAGAAACGATTTG |
| | | CTCTCTCTTCGTCTGGCTTTTCACTTTATATCGCACGGAA |
| | | TCAATGGATGGAACTCCTAAAGCTCCTAACTTCGATGATT |
| | | TGCTAGCCATGACTCTGTGGGACATTTTCTTGCATCTCGT |
| | | TTGTAACCTGTCTGTTCCTACACTAAGTTTATGAGAGGCT |
| | | ACTTTGGATTCTAGCCTCGGTGGTAAAGTGGGAGATAACA |
| | | ACGGCATAAGGCAAGAACCAGAAGTACCATAACGGTCTGG |
| | | TAAAGTTGGTGATAACTTAATTGGAAGAGTGTAAGTAAGA |
| | | CGTGGCTTGTAATAAGGCTTTCCATCAAAAAGGTTCTCCG |
| | | GGTTGGAGTTTGTGAGGCTCACATCTTTGATCAGTCTTTC |
| | | AATATAAATTGGTAACGTTGATGACAATGCCGGAGGTAAT |
| | | TTCTGTAGTTGTTGATATACGCAGATAACAGATTCAAATC |
| | | TCCATTGGTTTTCATCATTGTGGCTTAAATTAGATCAGAA |
| | | CATGGTAGTATTTAAAAATGGATCTCTTTGCAGATTTACT |
| | | CAATATAGCGAAAAAAGGAGACATTCGTTACAAAATATGA |
| | | AGATAATTCGCCTCATAACTCGATTAATCAAAACAGACGG |
| | | TCCAGTTCTTCTTTTGGTAGT |
| GBP2 promoter | SEQ ID NO: 40 | ATCTGTACTGGTACTGACAAAGGTTATCCAGAATCCGAGA |
| | | CATTTCAACAACAGAGATTCCAGGCTTCAAAACATCCATT |
| | | TTATCACCAATATCTAGTAATGCTTGCAACAATTCTGGAT |
| | | ACTTCTTCTGTGTAACCAAATCTCTTATAAACTGAACAGC |
| | | TTTCTGTACGTTGTCGTCAGTAGTTGGATCAACCTCAGTG |
| | | GTGACCTGGCCTATCGGTTTTCCAAAAGACTTGTTTATCA |
| | | CGTCCGAAAGCTCCCATTTTTGCAGATGCGCAACTTTAAA |
| | | AGGCCTGGCTTGAACATTTGCATCTCTTGTTGTGTGTTCT |
| | | TTGAGAAAATATTCATCGATCTGGGTGCTTCCAACGACAG |
| | | AAGATACTCTTCTGAGACCAGAAAGTCCCCAGCCATGCTT |
| | | CCTAATTACAAAATATTTGTAGGAAGATCCCTGATTAGGA |
| | | CAAAGTTGTCTTCTCATGAGTTCAACTGAAACTGGGGCTC |
| | | AAACGGATTATGAAAGGGGTGATTAAAGGTTTTCCTAGCC |
| | | TTACTTTCCAAATGTCGACCGAGACGAACATTTAAAATCC |
| | | TAACATCAGAAATTTCTATCCTTAATCTCATTGATGGTTA |
| | | GTACACTTCGCAGAGTCTCCACATTTGCAGACCCTCCTGG |
| | | ATAACCAAAGCTTATCTAACAGCGGCATTGGACCTTTGAA |
| | | AAGACCCTC |
| DAS1 promoter | SEQ ID NO: 41 | AAATCTGAACACGATGAAACCTCCCCGTAGATTCCACCGC |
| | | CCCGTTACTTTTTTGGGCAATCCCGTTGATAAGATCCATT |
| | | TTAGAGTTGTTTCTGAAAGGATTACAGGCGTTGAAGGGTC |
| | | AGAGAGATGCCAGAGAACAGACCAATTGGTAGTTTGCTAA |
| | | AGTGGACGTCTGGCAGGTGCTCTATCGTGTTCTTTATTTA |
| | | GGGCGTTACACTTAGTAGGATTACGTAACAATTTGGCTTA |
| | | ACCTTCTAAGTTAGAAAGAAACCAAGAGGGGTCCTCTTTA |
| | | ACGTTCAGCAGTATCTAAAACACAAAACCTGCCCTCATAA |
| | | TACATCATTCTATCTGTCAAGCTGTGCTACCCCACAGAAA |
| | | TACCCCCAAGAGTTAAAGTGAAAAGAAAAGCTAAATCTGT |
| | | TAGACTTCACCCCATAACAAACTTGATAGTTCCTGTAGCC |
| | | AATGAAAGTTAACCCCATTCAATGTTCCGAGATCTAGTAT |
| | | GCTTGCTCCTATAAGGAACGAAGGGTTCCAGCTTCCTTAC |
| | | CCCATCAATGGAAATCTCCTATTTACCCCCCACTGGAA |
| | | AGATCCGTCCGAACGAACGGATAATAGAAAAAAGAAATTC |
| | | GGACAAAATAGAACACTTATTTAGCCAATGAAATCCATTT |
| | | CCAGCATCTCCTTCAACTGCCGTTCCATCCCCTTTGTTGA |
| | | GCTACACCATCGTCAGCCAGTACCGAATAGGAAACTTAAC |
| | | CGATATCTTGGAGAATTCTAATGCGCGAATGAGTTTAGCC |
| | | TAGATATCCTTAGTGAAGGGTTGTTCCGATACTTCTCCAC |
| | | ATTCAGTCATTTCAGATGGGCAGCATTGTTATCATGAAGA |
| | | AACGGAAACGGGCAGTAAGGGTTAACCGCCAAATTATATA |
| | | AAGACAACATGTCCCCAGTTTAAAGTTTTTCTTTCCTATT |
| | | CTTGTATCCTGAGTGACCGTTGTGTTTAAAATAACAAGTT |
| | | CGTTTTAACTTAAGACCAAAACCAGTTACAACAAATTATT |
| | | CCCCAACTAAACACTAAAGTTCACTCTTATCAAACTATCA |
| | | AACATCAAAG |
| Methanol inducible | SEQ ID NO: 42 | CTTCCCCATTTCACTGACAGTTTGTAGAAA |
| promoter | | TAGGGCAACAATTGATGCAAATCGATTTTCAACGCATTGG |
| | | TTTTGATAGCATTGATGATCTTGGAGCTGTAAAAGTCCGG |
| | | CTGGATAAGCTCAATGAAATAGGTTGGTTGATCTGGATCT |
| | | TCTTTTGGGTCATTTTGTTCGCTCTGTATTTCACAAATTG |
| | | CCAGAATCTCTGCCAACCACAGTGGTAGGTCCAACTTGGT |
| | | GTTCTGAATCACAGGCTTCCCCGGGTTGTTCTCTAAATAA |
| | | CCGAGGCCCGGCACAGAAATCGTAAACCGACACGGTATCT |
| | | TTTGTCCGTCCGCCAGTATCTCATCAAGGTCGTAGTAGCC |
| | | CATGATGAGTATCAAAGGGGATTTGGTTATGCGATGCAAC |
| | | GAGAGATTGTTTATCCCAGATGCTGATGTAAAAACCTTAA |
| | | CCAGCGTGACAGTAGAAATAAGACACGTTAAAATTACCCG |
| | | CGCTTCCCTAACAATTGGCTCTGCCTTTCGGCAAGTTTCT |
| | | AACTGCCCTCCCCTCTCACATGCACCACGAACTTACCGTT |
| | | CGCTCCTAGCAGAACCACCCCAAAGTTTAATCAGGACCGC |
| | | ATTTTAGCCTATTGCTGTAGAACCCCACAACATAACCTGG |
| | | TCCAGAGCCAGCCCTTTATATATGGTAAATCCCGTTTGAA |
| | | CTTCGAAGTGGAATCGGAATTTTTACATCAAAGAAACTGA |
| | | TACTGAAACTTTTGGCTTCGACTTGGACTTTCTCTTAATC |
| | | GAATTCGT |
| GCW14 promoter | SEQ ID NO: 43 | CAGGTGAACCCACCTAACTATTTTTAACTGGCATCCAGTG |
| | | AGCTCGCTGGGTGAAAGCCAACCATCTTTTGTTTCGGGGA |
| | | ACCGTGCTCGCCCCGTAAAGTTAATTTTTTTTTCCCGCGC |
| | | AGCTTTAATCTTTCGGCAGAGAAGGCGTTTTCATCGTAGC |
| | | GTGGGAACAGAATAATCAGTTCATGTGCTATACAGGCACA |
| | | TGGCAGCAGTCACTATTTTGCTTTTTAACCTTAAAGTCGT |
| | | TCATCAATCATTAACTGACCAATCAGATTTTTTGCATTTG |
| | | CCACTTATCTAAAAATACTTTTGTATCTCGCAGATACGTT |
| | | CAGTGGTTTCCAGGACAACACCCAAAAAAAGGTATCAATG |
| | | CCACTAGGCAGTCGGTTTTATTTTTGGTCACCCACGCAAA |
| | | GAAGCACCCACCTCTTTTAGGTTTTAAGTTGTGGGAACAG |
| | | TAACACCGCCTAGAGCTTCAGGAAAAACCAGTACCTGTGA |
| | | CCGCAATTCACCATGATGCAGAATGTTAATTTAAACGAGT |
| | | GCCAAATCAAGATTTCAACAGACAAATCAATCGATCCATA |
| | | GTTACCCATTCCAGCCTTTTCGTCGTCGAGCCTGCTTCAT |
| | | TCCTGCCTCAGGTGCATAACTTTGCATGAAAAGTCCAGAT |
| | | TAGGGCAGATTTTGAGTTTAAAATAGGAAATATAAACAAA |
| | | TATACCGCGAAAAAGGTTTGTTTATAGCTTTTCGCCTGGT |
| | | GCCGTACGGTATAAATACATACTCTCCTCCCCCCCCTGGT |
| | | TCTCTTTTTCTTTTGTTACTTACATTTTACCGTTCCGT |
| FDH1 promoter | SEQ ID NO: 44 | AAATAAATGGCAGAAGGATCAGCCTGGACGAAGCAACCAG |
| | | TTCCAACTGCTAAGTAAAGAAGATGCTAGACGAAGGAGAC |
| | | TTCAGAGGTGAAAAGTTTGCAAGAAGAGAGCTGCGGGAAA |
| | | TAAATTTTCAATTTAAGGACTTGAGTGCGTCCATATTCGT |
| | | GTACGTGTCCAACTGTTTTCCATTACCTAAGAAAAACATA |
| | | AAGATTAAAAAGATAAACCCAATCGGGAAACTTTAGCGTG |
| | | CCGTTTCGGATTCCGAAAAACTTTTGGAGCGCCAGATGAC |
| | | TATGGAAAGAGGAGTGTACCAAAATGGCAAGTCGGGGGCT |
| | | ACTCACCGGATAGCCAATACATTCTCTAGGAACCAGGGAT |
| | | GAATCCAGGTTTTTGTTGTCACGGTAGGTCAAGCATTCAC |
| | | TTCTTAGGAATATCTCGTTGAAAGCTACTTGAAATCCCAT |
| | | TGGGTGCGGAACCAGCTTCTAATTAAATAGTTCGATGATG |
| | | TTCTCTAAGTGGGACTCTACGGCTCAAACTTCTACACAGC |
| | | ATCATCTTAGTAGTCCCTTCCCAAAACACCATTCTAGGTT |
| | | TCGGAACGTAACGAAACAATGTTCCTCTCTTCACATTGGG |
| | | CCGTTACTCTAGCCTTCCGAAGAACCAATAAAAGGGACCG |
| | | GCTGAAACGGGTGTGGAAACTCCTGTCCAGTTTATGGCAA |
| | | AGGCTACAGAAATCCCAATCTTGTCGGGATGTTGCTCCTC |
| | | CCAAACGCCATATTGTACTGCAGTTGGTGCGCATTTTAGG |
| | | GAAAATTTACCCCAGATGTCCTGATTTTCGAGGGCTACCC |
| | | CCAACTCCCTGTGCTTATACTTAGTCTAATTCTATTCAGT |
| | | GTGCTGACCTACACGTAATGATGTCGTAACCCAGTTAAAT |
| | | GGCCGAAAAACTATTTAAGTAAGTTTATTTCTCCTCCAGA |
| | | TGAGACTCTCCTTCTTTTCTCCGCTAGTTATCAAACTATA |
| | | AACCTATTTTACCTCAAATACCTCCAACATCACCCACTTA |
| | | AACAGAATT |
| FBA1 promoter | SEQ ID NO: 45 | TGCTTAAGTAATTGAAAACAGTGTTGTGATTATATAAGCA |
| | | TGGTATTTGAATAGAACTACTGGGGTTAACTTATCTAGTA |
| | | GGATGGAAGTTGAGGGAGATCAAGATGCTTAAAGAAAAGG |
| | | ATTGGCCAATATGAAAGCCATAATTAGCAATACTTATTTA |
| | | ATCAGATAATTGTGGGGCATTGTGACTTGACTTTTACCAG |
| | | GACTTCAAACCTCAACCATTTAAACAGTTATAGAAGACGT |
| | | ACCGTCACTTTTGCTTTTAATGTGATCTAAATGTGATCAC |
| | | ATGAACTCAAACTAAAATGATATCTTTTACTGGACAAAAA |
| | | TGTTATCCTGCAAACAGAAAGCTTTCTTCTATTCTAAGAA |
| | | GAACATTTACATTGGTGGGAAACCTGAAAACAGAAAATAA |
| | | ATACTCCCCAGTGACCCTATGAGCAGGATTTTTGCATCCC |
| | | TATTGTAGGCCTTTCAAACTCACACCTAATATTTCCCGCC |
| | | ACTCACACTATCAATGATCACTTCCCAGTTCTCTTCTTCC |
| | | CCTATTCGTACCATGCAACCCTTACACGCCTTTTCCATTT |
| | | CGGTTCGGATGCGACTTCCAGTCTGTGGGGTACGTAGCCT |
| | | ATTCTCTTAGCCGGTATTTAAACATACAAATTCACCCAAA |
| | | TTCTACCTTGATAAGGTAATTGATTAATTTCATAAATGAA |
| | | TTCGCG |
| GAP promoter | SEQ ID NO: 46 | TTTTTGTAGAAATGTCTTGGTGTCCTCGTCCAATCAGGTA |
| | | GCCATCTCTGAAATATCTGGCTCCGTTGCAACTCCGAACG |
| | | ACCTGCTGGCAACGTAAAATTCTCCGGGGTAAAACTTAAA |
| | | TGTGGAGTAATGGAACCAGAAACGTCTCTTCCCTTCTCTC |
| | | TCCTTCCACCGCCCGTTACCGTCCCTAGGAAATTTTACTC |
| | | TGCTGGAGAGCTTCTTCTACGGCCCCCTTGCAGCAATGCT |
| | | CTTCCCAGCATTACGTTGCGGGTAAAACGGAGGTCGTGTA |
| | | CCCGACCTAGCAGCCCAGGGATGGAAAAGTCCCGGCCGTC |
| | | GCTGGCAATAATAGCGGGCGGACGCATGTCATGAGATTAT |
| | | TGGAAACCACCAGAATCGAATATAAAAGGCGAACACCTTT |
| | | CCCAATTTTGGTTTCTCCTGACCCAAAGACTTTAAATTTA |
| | | ATTTATTTGTCCCTATTTCAATCAATTGAACAACTAT |
| PGK promoter | SEQ ID NO: 47 | AAATAGCAGTTTGCGGTTTCTTGATTTCATGGGGGGAACA |
| | | AACAATAGTGTTGCCTTAATTCTAATTGGCATTGTTGCTT |
| | | GGAATCGAAATTGGGGGATAACGTCATATCTGAAAAGTAA |
| | | ACAACTTCGGGAAATCAGGCTGTTTGAATGGCTTGGAAGC |
| | | GAGATAGAAAGGGGATAGCGAGATAGAGGGGGCGGAGTAG |
| | | ACGAAGGGTGTTAAACTGCTGAAATCTCTCAATCTGGAAG |
| | | AAACGGAATAAATTAACTCCTTGCGATAATAAAATCCGAG |
| | | TCCGTTATGACCCCACACCGTGTTGACCACGGCATACCCC |
| | | ATGGAATCTGGTACAAAGCGTCAGTCTTGAAGACACCATC |
| | | ACGTGTAGGAGACTGATTGTCTGACCGTCCAGCAAAAAGG |
| | | GCATTATAAATCTTGCTGTTAAAGGGGTGAGGGGAGATGC |
| | | AGGTTGTTCTTTTATTCGCCTTGAACTTTTTAATTTTCCC |
| | | GGGGTTGCGGAGCGTGAACAGTTAGCCCGATCTGATAGCT |
| | | TGCAAGATTCAACAGTTTATCCACTACAGGTCAGAGAGAT |
| | | CGCCGCAGAAGAAATGCTCGTCTCGTGTTCCAGCACACAT |
| | | ACTGGTGAAGTCGTTATTTTGCCGAAGGGGGGGTAATAAG |
| | | GTTATGCACCCCCTCTCCACACCCCAGAATCATTTTTTAG |
| | | CTGGGTTCAAGGCATTAGACTTTGCACATTTTTCCCTTAA |
| | | ACACCCTTGAAACGCGGATAAACAGTTGCATGTGCATCCT |
| | | AAAACTAGGTGAGATGCGTACTCCGTGCTCCGATAATAAC |
| | | AGTGGTGTTGGGGTTGCTGCTAGCTCACGCACTCCGTTCT |
| | | TTTTTTTCAACCAGCAAAATTCGATGGGGAGAAACTTGGG |
| | | GTACTTTGCCGACTCCTCCACCATGCTGGTATATAAATAA |
| | | TACTCGCCCACTTTTCGTTTGCTGCTTTTATATTTCATAG |
| | | ACTGAAAAAGACTCTTCTTCTACTTTTTCATAATATATCT |
| | | CAGATATCACTACTATAG |
| TEFg_ promoter | SEQ ID NO: 48 | GCGATTTAAATTCGCGAAAGAACAGCCTAATAAACTCCGA |
| | | AGCATGATGGCCTCTATCCGGAAAACGTTAAGAGATGTGG |
| | | CAACAGGAGGGCACATAGAATTTTTAAAGACGCTGAAGAA |
| | | TGCTATCATAGTCCGTAAAAATGTGATAGTACTTTGTTTA |
| | | GTGCGTACGCCACTTATTCGGGGCCAATAGCTAAACCCAG |
| | | GTTTGCTGGCAGCAAATTCAACTGTAGATTGAATCTCTCT |
| | | AACAATAATGGTGTTCAATCCCCTGGCTGGTCACGGGGAG |
| | | GACTATCTTGCGTGATCCGCTTGGAAAATGTTGTGTATCC |
| | | CTTTCTCAATTGCGGAAAGCATCTGCTACTTCCCATAGGC |
| | | ACCAGTTACCCAATTGATATTTCCAAAAAAGATTACCATA |
| | | TGTTCATCTAGAAGTATAAATACAAGTGGACATTCAATGA |
| | | ATATTTCATTCAATTAGTCATTGACACTTTCATCAACTTA |
| | | CTACGTCTTATTCAACAATGAATTCGCG |
| AOX1 terminator | SEQ ID NO: 53 | TCAAGAGGATGTCAGAATGCCATTTGCCTGAGAGATGCAG |
| | | GCTTCATTTTTGATACTTTTTTATTTGTAACCTATATAGT |
| | | ATAGGATTTTTTTTGTCATTTTGTTTCTTCTCGTACGAGC |
| | | TTGCTCCTGATCAGCCTATCTCGCAGCAGATGAATATCTT |
| | | GTGGTAGGGGTTTGGGAAAATCATTCGAGTTTGATGTTTT |
| | | TCTTGGTATTTCCCACTCCTCTTCAGAGTACAGAAGATTA |
| | | AGTGAAACCTTCGTTTGTGCG |
| TDH3 terminator | SEQ ID NO: 54 | TCGATTTGTATGTGAAATAGCTGAAATTCGAAAATTTCAT |
| | | TATGGCTGTATCTACTTTAGCGTATTAGGCATTTGAGCAT |
| | | TGGCTTGAACAATGCGGGCTGTAGTGTGTCACCAAAGAAA |
| | | CCATTCGGGTTCGGATCTGGAAGTCCTCATCACGTGATGC |
| | | CGATCTCGTGTATTTTATTTTCAGATAACACCTGAAGACT |
| | | TT |
| RPS25A terminator | SEQ ID NO: 55 | ATTAGTGTACATCTGATAATATAGTACTACCACGTATGAT |
| | | AATGTAGAGAATAGTCTTCCTTGTCGAGTGTGTTTGCAGT |
| | | TTTCTTGAGTTTCAAGGTTTAAATGCTGGTATATTAGTTC |
| | | ATCGAAGGTTTCAGCCAATAGCACCTTAAATCAATCAAAC |
| | | TAATTCGACTCTTACGAAAGAGCCTACTGTGTTTAGTATC |
| | | GAAGTCGTTTACCTTTCATGTTGAATAGCTTCCTCTCTGA |
| | | CCCTAACATTTCAAGATCCTCCTAAAGTTACCCGGATTGT |
| | | GAAATTCTAATGATCCACCTGCCCAATGCATTTTTTCTTT |
| | | ATTCAGTTTACCTTTTTTACCTAATATACGAGCTTGTTAA |
| | | AGTAAGTGGCACTGCAATACTAGGCTTATTGTTGATATTA |
| | | TGATGAATCGTTTTCACAAACTTGATTTCCTGTGAACTCA |
| | | CCATGTACTAAGGAAAAAAACATGCATCACCATCTGAATA |
| | | TTTGAC |
| RPL2A terminator | SEQ ID NO: 56 | ACTATGTAACTAACGAAACAGCATGTACTAATAGAACCGT |
| | | ATCGAGAATATTTATTTAGGTGAGTAGTAGGAGTGAACCA |
| | | GACAGTCAATTTAGTGAGCTGTCCCAGCTTTTGTGCATTC |
| | | CAGAATTGCCGGTCAAATTGGTTATGGGTTATGGGGCTTT |
| | | TCCGATTGAGGTTCAGTTTCTGCGGTTATCTCTTTCTTGA |
| | | CCTGGTCTTTTACAGGCTGTTCTTTCTCCCCATGATTATT |
| | | CTTTAGCTGAAGATACCGCTTAGCCTGATAATGTCGTCGT |
| | | TTTGTAATCAAAATCTTTAGTTGGGCATCGTCTGAGGTTT |
| | | CCTTTGGCTTCTGGGGTTGTTAGTAGGAACGTAGGAACCA |
| | | TAGTAACTTTTACACATACATTCTTATGATTGCGAAGTAA |
| | | GCTGAGTCTGCTGCTTGGCTCCCGAAGTACTTTCTCTTTC |
| | | TCTACCGGTTGATTCTCCTTCTGGTGCTCCTAAACGATTG |
| | | TGTTAGAAGGGATTGAC |
| Signal Peptide | SEQ ID NO: 57 | MFTPVRRRVRTAALALSAAAALVLGSTAASGASATPSPAP |
| | | AP |
| Signal Peptide | SEQ ID NO: 58 | MKLSTVLLSAGLASTTLA |
| Signal Peptide | SEQ ID NO: 59 | MRFPSIFTAVLFAASSALA |
| Signal Peptide | SEQ ID NO: 60 | MVSLRSIFTSSILAAGLTRAHG |
| Signal Peptide | SEQ ID NO: 61 | MKFPVPLLFLLQLFFIIATQG |
| Signal Peptide | SEQ ID NO: 62 | MQVKSIVNLLLACSLAVA |
| Signal Peptide | SEQ ID NO: 63 | MQFNWNIKTVASILSALTLAQA |
| Signal Peptide | SEQ ID NO: 64 | MYRNLIIATALTCGAYSAYVPSEPWSTLTPDASLESALKD |
| | | YSQTFGIAIKSLDADKIKR |
| Signal Peptide | SEQ ID NO: 65 | MNLYLITLLFASLCSAITLPKR |
| Signal Peptide | SEQ ID NO: 66 | MFEKSKFVVSFLLLLQLFCVLGVHG |
| Signal Peptide | SEQ ID NO: 67 | MQFNSVVISQLLLTLASVSMG |
| Signal Peptide | SEQ ID NO: 68 | MKSQLIFMALASLVASAPLEHQQQHHKHEKR |
| Signal Peptide | SEQ ID NO: 69 | MKFAISTLLIILQAAAVFA |
| Signal Peptide | SEQ ID NO: 70 | MKLLNFLLSFVTLFGLLSGSVFA |
| Signal Peptide | SEQ ID NO: 71 | MIFNLKTLAAVAISISQVSA |
| Signal Peptide | SEQ ID NO: 72 | MKISALTACAVTLAGLAIAAPAPKPEDCTTTVQKRHQHKR |
| Signal Peptide | SEQ ID NO: 73 | MSYLKISALLSVLSVALA |
| Signal Peptide | SEQ ID NO: 74 | MLSTILNIFILLLFIQASLQ |
| Signal Peptide | SEQ ID NO: 75 | MKLSTNLILAIAAASAVVSAAPVAPAEEAANHLHKR |
| Signal Peptide | SEQ ID NO: 76 | MFKSLCMLIGSCLLSSVLA |
| Signal Peptide | SEQ ID NO: 77 | MKLAALSTIALTILPVALA |
| Signal Peptide | SEQ ID NO: 78 | MSFSSNVPQLFLLLVLLTNIVSG |
| Signal Peptide | SEQ ID NO: 79 | MQLQYLAVLCALLLNVQSKNVVDFSRFGDAKISPDDTDLE |
| | | SRERKR |
| Signal Peptide | SEQ ID NO: 80 | MKIHSLLLWNLFFIPSILG |
| Signal Peptide | SEQ ID NO: 81 | MSTLTLLAVLLSLQNSALA |
| Signal Peptide | SEQ ID NO: 82 | MINLNSFLILTVTLLSPALALPKNVLEEQQAKDDLAKR |
| Signal Peptide | SEQ ID NO: 83 | MFSLAVGALLLTQAFG |
| Signal Peptide | SEQ ID NO: 84 | MKILSALLLLFTLAFA |
| Signal Peptide | SEQ ID NO: 85 | MKVSTTKFLAVFLLVRLVCA |
| Signal Peptide | SEQ ID NO: 86 | MQFGKVLFAISALAVTALG |
| Signal Peptide | SEQ ID NO: 87 | MWSLFISGLLIFYPLVLG |
| Signal Peptide | SEQ ID NO: 88 | MRNHLNDLVVLFLLLTVAAQA |
| Signal Peptide | SEQ ID NO: 89 | MFLKSLLSFASILTLCKA |
| Signal Peptide | SEQ ID NO: 90 | MFVFEPVLLAVLVASTCVTA |
| Signal Peptide | SEQ ID NO: 91 | MFSPILSLEIILALATLQSVFA |
| Signal Peptide | SEQ ID NO: 92 | MIINHLVLTALSIALA |
| Signal Peptide | SEQ ID NO: 93 | MLALVRISTLLLLALTASA |
| Signal Peptide | SEQ ID NO: 94 | MRPVLSLLLLLASSVLA |
| Signal Peptide | SEQ ID NO: 95 | MVLIQNFLPLFAYTLFFNQRAALA |
| Signal Peptide | SEQ ID NO: 96 | MVSLTRLLITGIATALQVNA |
| Signal Peptide | SEQ ID NO: 97 | MIFDGTTMSIAIGLLSTLGIGAEA |
| Signal Peptide | SEQ ID NO: 98 | MVLVGLLTRLVPLVLLAGTVLLLVFVVLSGG |
| Signal Peptide | SEQ ID NO: 99 | MLSILSALTLLGLSCA |
| Signal Peptide | SEQ ID NO: 100 | MRLLHISLLSIISVLTKANA |
| Signal Peptide | SEQ ID NO: 101 | MRFPSIFTAVLFAASSALAAPVNTTTEDETAQIPAEAVIG |
| | | YLDLEGDFDVAVLPFSNSTNNGLLFINTTIASIAAKEEGV |
| | | SLDKREAEA |
| Signal Peptide | SEQ ID NO: 102 | MFKSVVYSILAASLANA |
| Signal Peptide | SEQ ID NO: 103 | MLLQAFLFLLAGFAAKISA |
| Signal Peptide | SEQ ID NO: 104 | MASSNLLSLALFLVLLTHANS |
| Signal Peptide | SEQ ID NO: 105 | MNIFYIFLFLLSFVQGLEHTHRRGSLVKR |
| Signal Peptide | SEQ ID NO: 106 | MLIIVLLFLATLANSLDCSGDVFFGYTRGDKTDVHKSQAL |
| | | TAVKNIKR |
| Signal Peptide | SEQ ID NO: 107 | MESVSSLFNIFSTIMVNYKSLVLALLSVSNLKYARGMPTS |
| | | ERQQGLEER |
| Signal Peptide | SEQ ID NO: 108 | MFAFYFLTACISLKGVFG |
| Signal Peptide | SEQ ID NO: 109 | MRFSTTLATAATALFFTASQVSA |
| Signal Peptide | SEQ ID NO: 110 | MKFAYSLLLPLAGVSASVINYKR |
| Signal Peptide | SEQ ID NO: 111 | MKFFAIAALFAAAAVAQPLEDR |
| Signal Peptide | SEQ ID NO: 112 | MQFFAVALFATSALA |
| Signal Peptide | SEQ ID NO: 113 | MKWVTFISLLFLFSSAYSRGVFRR |
| Signal Peptide | SEQ ID NO: 114 | MRSLLILVLCFLPLAALG |
| Signal Peptide | SEQ ID NO: 115 | MKVLILACLVALALA |
| Signal Peptide | SEQ ID NO: 116 | MFNLKTILISTLASIAVA |
| Signal Peptide | SEQ ID NO: 117 | MYRKLAVISAFLATARAQSA |
| WT | SEQ ID NO: 118 | MRFPSIFTAVLFAASSALAAPVNTTTEDETAQIPAEAVIG |
| | | YLDLEGDFDVAVLPFSNSTNNGLLFINTTIASIAAKEEGV |
| | | QLDKR |
| App3 | SEQ ID NO: 119 | MRFPPIFTAALFAASSALAAPANTTTEDETAQIPAEAVIG |
| | | YLDSEGDSDVAVLPFSNSTNNGLSFINTTIASIAAKEEGV |
| | | QLDKR |
| App8 | SEQ ID NO: 120 | MRFPSIFTAVLFAASSALAAPANTTTEDETAQIPAEAVIS |
| | | YSDLEGDFDAAALPLSNSTNNGLSSTNTTIASIAAKEEGV |
| | | QLDKR |
| App9 | SEQ ID NO: 121 | MRPPSIFTAVLFAASSALAAPANTTTEDETTQIPAEAVAT |
| | | YLDLEGDVDVAVLPFSSSTNNGLSFINTTIASIAAKEEGV |
| | | QLDKR |
| App10 | SEQ ID NO: 122 | MRFPSIFTAALFAASSALAAPANTTTEGETAQTPAEAVIG |
| | | YRDLEGDFDVAVLPFPNSTNNGLLFTNTTTASIAAKEEGV |
| | | QLDKR |
| appS1 | SEQ ID NO: 123 | MRFPSIFTAVLLAAPSALAAPANATTEDEAAQIPAEAVIG |
| | | YLDLEGDFDAAVLPFSNSTNNGLLSINTTIASIAAKEEGV |
| | | QLDKR |
| appS4 | SEQ ID NO: 124 | MRFPSIFTAVVFAASSALAAPANTTAEDETAQIPAEAVIG |
| | | YLGLEGDSDVAALPLSDSTNNGSLSTNTTIASIAAKEEGV |
| | | QLDKR |
| appS6 | SEQ ID NO: 125 | MRLPSIFTAAVFAASSALAAPANTTTEDETAQIPAEAAIG |
| | | YLDLEGDSDVAVLPLSNSTNNGLLFINTTIASIAAKEEGV |
| | | QLDKR |
| appS8 | SEQ ID NO: 126 | MRFPSIFTAVLFAASSALAAPANTTTEDETAQIPAEAVIG |
| | | YLDLEGDFDVAVLPFSNSTNDGLSFINTTTASIAAKEEGV |
| | | QLDKR |
| a-Factor | SEQ ID NO: 127 | MRFPSIFTAVLFAASSALAAPVNTTTEDETAQIPA |
| PpScw11p | SEQ ID NO: 128 | MLSTILNIFILLLFIQASLQAPIPVVTKYVTEGIAVV |
| PpDse4p | SEQ ID NO: 129 | MSFSSNVPQLFLLLVLLTNIVSGAVISVWSTSKVTK |
| PpExg1p | SEQ ID NO: 130 | MNLYLITLLFASLCSAITLPKRDIIWDYSSEKIMG |
| a-EGFP | SEQ ID NO: 131 | MRFPSIFTAVLFAASSALAAPVNTTTEDETAQIPA |
| S-EGFP | SEQ ID NO: 132 | MLSTILNIFILLLFIQASLQEFDYKDDDDKMVSKG |
| D-EGFP | SEQ ID NO: 133 | MSFSSNVPQLFLLLVLLTNIVSGEFDYKDDDDKMV |
| E-EGFP | SEQ ID NO: 134 | MNLYLITLLFASLCSAEFDYKDDDDKMVSKGEELF |
| a-CALB | SEQ ID NO: 135 | MRFPSIFTAVLFAASSALAAPVNTTTEDETAQIPA |
| S-CALB | SEQ ID NO: 136 | MLSTILNIFILLLFIQASLQEFLPSGSDPAFSQPK |
| D-CALB | SEQ ID NO: 137 | MSFSSNVPQLFLLLVLLTNIVSGEFLPSGSDPAFS |
| E-CALB | SEQ ID NO: 138 | MNLYLITLLFASLCSAEFLPSGSDPAFSQPKSVLD |
| Amylase (AA) | SEQ ID NO: 139 | MVAWWSLFLYGLQVAAPALAAEVDCSRFPNATDKEGKDVL |
| | | VCNKDLRPICGTDGVTYTNDCLLCAYSIEFGTNISKEHDG |
| | | ECKETVPMNCSSYANTTSEDGKVMVLCNRAFNPVCGTDGV |
| | | TYDNECLLCAHKVEQGASVDKRHDGGCRKELAAVSVDCSE |
| | | YPKPDCTAEDRPLCGSDNKTYGNKCNFCNAVVESNGTLTL |
| | | SHFGKC |
| Alpha K (AK) | SEQ ID NO: 140 | MRFPSIFTAVLFAASSALAAPVNTTTEDELEGDFDVAVLP |
| | | FSASIAAKEEGVSLEKRAEVDCSRFPNATDKEGKDVLVCN |
| | | KDLRPICGTDGVTYTNDCLLCAYSIEFGTNISKEHDGECK |
| | | ETVPMNCSSYANTTSEDGKVMVLCNRAFNPVCGTDGVTYD |
| | | NECLLCAHKVEQGASVDKRHDGGCRKELAAVSVDCSEYPK |
| | | PDCTAEDRPLCGSDNKTYGNKCNFCNAVVESNGTLTLSHF |
| | | GKC |
| Alpha T (AT) | SEQ ID NO: 141 | MRFPSIFTAVLFAASSALAAEVDCSRFPNATDKEGKDVLV |
| | | CNKDLRPICGTDGVTYTNDCLLCAYSIEFGTNISKEHDGE |
| | | CKETVPMNCSSYANTTSEDGKVMVLCNRAFNPVCGTDGVT |
| | | YDNECLLCAHKVEQGASVDKRHDGGCRKELAAVSVDCSEY |
| | | PKPDCTAEDRPLCGSDNKTYGNKCNFCNAVVESNGTLTLS |
| | | HFGKC |
| Lysozyme (LZ) | SEQ ID NO: 142 | MLGKNDPMCLVLVLLGLTALLGICQGAEVDCSRFPNATDK |
| | | EGKDVLVCNKDLRPICGTDGVTYTNDCLLCAYSIEFGTNI |
| | | SKEHDGECKETVPMNCSSYANTTSEDGKVMVLCNRAFNPV |
| | | CGTDGVTYDNECLLCAHKVEQGASVDKRHDGGCRKELAAV |
| | | SVDCSEYPKPDCTAEDRPLCGSDNKTYGNKCNFCNAVVES |
| | | NGTLTLSHFGKC |
| Killer Protein (KP) | SEQ ID NO: 143 | MTKPTQVLVRSVSILFFITLLHLVVAAEVDCSRFPNATDK |
| | | EGKDVLVCNKDLRPICGTDGVTYTNDCLLCAYSIEFGTNI |
| | | SKEHDGECKETVPMNCSSYANTTSEDGKVMVLCNRAFNPV |
| | | CGTDGVTYDNECLLCAHKVEQGASVDKRHDGGCRKELAAV |
| | | SVDCSEYPKPDCTAEDRPLCGSDNKTYGNKCNFCNAVVES |
| | | NGTLTLSHFGKC |
| Invertase (IV) | SEQ ID NO: 144 | MLLQAFLFLLAGFAAKISAAEVDCSRFPNATDKEGKDVLV |
| | | CNKDLRPICGTDGVTYTNDCLLCAYSIEFGTNISKEHDGE |
| | | CKETVPMNCSSYANTTSEDGKVMVLCNRAFNPVCGTDGVT |
| | | YDNECLLCAHKVEQGASVDKRHDGGCRKELAAVSVDCSEY |
| | | PKPDCTAEDRPLCGSDNKTYGNKCNFCNAVVESNGTLTLS |
| | | HFGKC |
| Serum Albumin (SA) | SEQ ID NO: 145 | MKWVTFISLLFLFSSAYSAEVDCSRFPNATDKEGKDVLVC |
| | | NKDLRPICGTDGVTYTNDCLLCAYSIEFGTNISKEHDGEC |
| | | KETVPMNCSSYANTTSEDGKVMVLCNRAFNPVCGTDGVTY |
| | | DNECLLCAHKVEQGASVDKRHDGGCRKELAAVSVDCSEYP |
| | | KPDCTAEDRPLCGSDNKTYGNKCNFCNAVVESNGTLTLSH |
| | | FGKC |
| Glucoamyl (GA) | SEQ ID NO: 146 | MSFRSLLALSGLVCSGLAAEVDCSRFPNATDKEGKDVLVC |
| | | NKDLRPICGTDGVTYTNDCLLCAYSIEFGTNISKEHDGEC |
| | | KETVPMNCSSYANTTSEDGKVMVLCNRAFNPVCGTDGVTY |
| | | DNECLLCAHKVEQGASVDKRHDGGCRKELAAVSVDCSEYP |
| | | KPDCTAEDRPLCGSDNKTYGNKCNFCNAVVESNGTLTLSH |
| | | FGKC |
| Inulase (IN) - IC | SEQ ID NO: 147 | MKLAYSLLLPLAGVSAAEVDCSRFPNATDKEGKDVLVCNK |
| | | DLRPICGTDGVTYTNDCLLCAYSIEFGTNISKEHDGECKE |
| | | TVPMNCSSYANTTSEDGKVMVLCN |
| | | RAFNPVCGTDGVTYDNECLLCAHKVEQGASVDKRHDGGCR |
| | | KELAAVSVDCSEYPKPDCTAEDRPLCGSDNKTYGNKCNFC |
| | | NAVVESNGTLTLSHFGKC |
| Alpha KS (AKS) | SEQ ID NO: 148 | MRFPSIFTAVLFAASSALAAPVNTTTEDELEGDFDVAVLP |
| | | FSASIAAKEEGVSLEKREAEAAEVDCSRFPNATDKEGKDV |
| | | LVCNKDLRPICGTDGVTYTNDCLLCAYSIEFGTNISKEHD |
| | | GECKETVPMNCSSYANTTSEDGKVMVLCNRAFNPVCGTDG |
| | | VTYDNECLLCAHKVEQGASVDKRHDGGCRKELAAVSVDCS |
| | | EYPKPDCTAEDRPLCGSDNKTYGNKCNFCNAVVESNGTLT |
| | | LSHFGKC |
| Ovomucoid signal | SEQ ID NO: 149 | MAMAGVFVLFSFVLCGFLPDAAFG |
| peptide | | |
| Lysozyme signal | SEQ ID NO: 150 | MRSLLILVLCFLPLAALG |
| peptide | | |
| Ovalbumin Signal | SEQ ID NO: 151 | MRFPSIFTAVLFAASSALAAPVNTTTEDETAQIPAEAVIG |
| Peptide | | YSDLEGDFDVAVLPFSNSTNNGLLFINTTIASIAAKEEGV |
| | | SLDKREAEA |
| Ovotransferrin Signal | SEQ ID NO: 152 | MKLILCTVLSLGIAAVCFA |
| Peptide | | |
| Bovine Lactoferrin | SEQ ID NO: 153 | MKLFVPALLSLGALGLCLA |
| Signal Peptide | | |
| Porcine Lactoferrin | SEQ ID NO: 154 | MKLFIPALLFLGTLGLCLA |
| Signal Peptide | | |
| Kid Lipase Signal | SEQ ID NO: 155 | MESKALLLLALSVWLQSLTVSHG |
| Peptide | | |
| Porcine Lipase | SEQ ID NO: 156 | MLLIWTLSLLLGAVLG |
| Signal Peptide | | |
| Ovomucoid | SEQ ID NO: 157 | AEVDCSRFPNATDKEGKDVLVCNKDLRPICGTDGVTYTND |
| (canonical) | | CLLCAYSIEFGTNISKEHDGECKETVPMNCSSYANTTSED |
| | | GKVMVLCNRAFNPVCGTDGVTYDNECLLCAHKVEQGASVD |
| | | KRHDGGCRKELAAVSVDCSEYPKPDCTAEDRPLCGSDNKT |
| | | YGNKCNFCNAVVESNGTLTLSHFGKC* |
| Ovomucoid | SEQ ID NO: 158 | AEVDCSRFPNATDMEGKDVLVCNKDLRPICGTDGVTYTND |
| | | CLLCAYSVEFGTNISKEHDGECKETVPMNCSSYANTTSED |
| | | GKVMVLCNRAFNPVCGTDGVTYDNECLLCAHKVEQGASVD |
| | | KRHDGGCRKELAAVSVDCSEYPKPDCTAEDRPLCGSDNKT |
| | | YGNKCNFCNAVVESNGTLTLSHFGKC* |
| Ovomucoid | SEQ ID NO: 159 | AEVDCSRFPNATDMEGKDVLVCNKDLRPICGTDGVTYTND |
| G162M F167A | | CLLCAYSVEFGTNISKEHDGECKETVPMNCSSYANTTSED |
| | | GKVMVLCNRAFNPVCGTDGVTYDNECLLCAHKVEQGASVD |
| | | KRHDGGCRKELAAVSVDCSEYPKPDCTAEDRPLCGSDNKT |
| | | YMNKCNACNAVVESNGTLTLSHFGKC* |
| Ovomucoid isoform 1 | SEQ ID NO: 160 | MAMAGVFVLFSFVLCGFLPDAAFGAEVDCSRFPNATDKEG |
| precursor full length | | KDVLVCNKDLRPICGTDGVTYTNDCLLCAYSIEFGTNISK |
| | | EHDGECKETVPMNCSSYANTTSEDGKVMVLCNRAFNPVCG |
| | | TDGVTYDNECLLCAHKVEQGASVDKRHDGGCRKELAAVSV |
| | | DCSEYPKPDCTAEDRPLCGSDNKTYGNKCNFCNAVVESNG |
| | | TLTLSHFGKC |
| Ovomucoid [Gallus | SEQ ID NO: 161 | MAMAGVFVLFSFVLCGFLPDAVFGAEVDCSRFPNATDMEG |
| gallus] | | KDVLVCNKDLRPICGTDGVTYTNDCLLCAYSVEFGTNISK |
| | | EHDGECKETVPMNCSSYANTTSEDGKVMVLCNRAFNPVCG |
| | | TDGVTYDNECLLCAHKVEQGASVDKRHDGGCRKELAAVSV |
| | | DCSEYPKPDCTAEDRPLCGSDNKTYGNKCNFCNAVVESNG |
| | | TLTLSHFGKC |
| Ovomucoid isoform 2 | SEQ ID NO: 162 | MAMAGVFVLFSFVLCGFLPDAAFGAEVDCSRFPNATDKEG |
| precursor [Gallus | | KDVLVCNKDLRPICGTDGVTYTNDCLLCAYSIEFGTNISK |
| gallus] | | EHDGECKETVPMNCSSYANTTSEDGKVMVLCNRAFNPVCG |
| | | TDGVTYDNECLLCAHKVEQGASVDKRHDGGCRKELAAVDC |
| | | SEYPKPDCTAEDRPLCGSDNKTYGNKCNFCNAVVESNGTL |
| | | TLSHFGKC |
| Ovomucoid [Gallus | SEQ ID NO: 163 | AEVDCSRFPNATDKEGKDVLVCNKDLRPICGTDGVTYNNE |
| gallus] | | CLLCAYSIEFGTNISKEHDGECKETVPMNCSSYANTTSED |
| | | GKVMVLCNRAFNPVCGTDGVTYDNECLLCAHKVEQGASVD |
| | | KRHDGECRKELAAVSVDCSEYPKPDCTAEDRPLCGSDNKT |
| | | YGNKCNFCNAVVESNGTLTLSHFGKC |
| Ovomucoid [Numida | SEQ ID NO: 164 | MAMAGVFVLFSFALCGFLPDAAFGVEVDCSRFPNATNEEG |
| meleagris] | | KDVLVCTEDLRPICGTDGVTYSNDCLLCAYNIEYGTNISK |
| | | EHDGECREAVPVDCSRYPNMTSEEGKVLILCNKAFNPVCG |
| | | TDGVTYDNECLLCAHNVEQGTSVGKKHDGECRKELAAVDC |
| | | SEYPKPACTMEYRPLCGSDNKTYDNKCNFCNAVVESNGTL |
| | | TLSHFGKC |
| PREDICTED: | SEQ ID NO: 165 | MQTITWRQPQGDHLRSRAPAATCRAGQYLTMAMAGIFVLF |
| Ovomucoid isoform | | SFALCGFLPDAAFGVEVDCSRFPNTTNEEGKDVLVCTEDL |
| X1 [Meleagris | | RPICGTDGVTHSECLLCAYNIEYGTNISKEHDGECREAVP |
| gallopavo] | | MDCSRYPNTTNEEGKVMILCNKALNPVCGTDGVTYDNECV |
| | | LCAHNLEQGTSVGKKHDGGCRKELAAVSVDCSEYPKPACT |
| | | LEYRPLCGSDNKTYGNKCNFCNAVVESNGTLTLSHFGKC |
| Ovomucoid | SEQ ID NO: 166 | VEVDCSRFPNTTNEEGKDVLVCTEDLRPICGTDGVTHSEC |
| [Meleagris gallopavo] | | LLCAYNIEYGTNISKEHDGECREAVPMDCSRYPNTTSEEG |
| | | KVMILCNKALNPVCGTDGVTYDNECVLCAHNLEQGTSVGK |
| | | KHDGECRKELAAVSVDCSEYPKPACTLEYRPLCGSDNKTY |
| | | GNKCNFCNAVVESNGTLTLSHFGKC |
| PREDICTED: | SEQ ID NO: 167 | MQTITWRQPQGDHLRSRAPAATCRAGQYLTMAMAGIFVLF |
| Ovomucoid isoform | | SFALCGFLPDAAFGVEVDCSRFPNTTNEEGKDVLVCTEDL |
| X2 [Meleagris | | RPICGTDGVTHSECLLCAYNIEYGTNISKEHDGECREAVP |
| gallopavo] | | MDCSRYPNTTNEEGKVMILCNKALNPVCGTDGVTYDNECV |
| | | LCAHNLEQGTSVGKKHDGGCRKELAAVDCSEYPKPACTLE |
| | | YRPLCGSDNKTYGNKCNFCNAVVESNGTLTLSHFGKC |
| Ovomucoid | SEQ ID NO: 168 | EYGTNISIKHNGECKETVPMDCSRYANMTNEEGKVMMPCD |
| [Bambusicola | | RTYNPVCGTDGVTYDNECQLCAHNVEQGTSVDKKHDGVCG |
| thoracicus] | | KELAAVSVDCSEYPKPECTAEERPICGSDNKTYGNKCNFC |
| | | NAVVYVQP |
| Ovomucoid | SEQ ID NO: 169 | VDCSRFPNTTNEEGKDVLACTKELHPICGTDGVTYSNECL |
| [Callipepla squamata] | | LCYYNIEYGTNISKEHDGECTEAVPVDCSRYPNTTSEEGK |
| | | VLIPCNRDFNPVCGSDGVTYENECLLCAHNVEQGTSVGKK |
| | | HDGGCRKEFAAVSVDCSEYPKPDCTLEYRPLCGSDNKTYA |
| | | SKCNFCNAVVIWEQEKNTRHHASHSVFFISARLVC |
| Ovomucoid [Colinus | SEQ ID NO: 170 | MLPLGLREYGTNTSKEHDGECTEAVPVDCSRYPNTTSEEG |
| virginianus] | | KVRILCKKDINPVCGTDGVTYDNECLLCSHSVGQGASIDK |
| | | KHDGGCRKEFAAVSVDCSEYPKPACMSEYRPLCGSDNKTY |
| | | VNKCNFCNAVVYVQPWLHSRCRLPPTGTSFLGSEGRETSL |
| | | LTSRATDLQVAGCTAISAMEATRAAALLGLVLLSSFCELS |
| | | HLCFSQASCDVYRLSGSRNLACPRIFQPVCGTDNVTYPNE |
| | | CSLCRQMLRSRAVYKKHDGRCVKVDCTGYMRATGGLGTAC |
| | | SQQYSPLYATNGVIYSNKCTFCSAVANGEDIDLLAVKYPE |
| | | EESWISVSPTPWRMLSAGA |
| Ovomucoid-like | SEQ ID NO: 171 | MSWWGIKPALERPSQEQSTSGQPVDSGSTSTTTMAGIFVL |
| isoform X2 [Anser | | LSLVLCCFPDAAFGVEVDCSRFPNTTNEEGKEVLLCTKDL |
| cygnoides domesticus] | | SPICGTDGVTYSNECLLCAYNIEYGTNISKDHDGECKEAV |
| | | PVDCSTYPNMTNEEGKVMLVCNKMFSPVCGTDGVTYDNEC |
| | | MLCAHNVEQGTSVGKKYDGKCKKEVATVDCSDYPKPACTV |
| | | EYMPLCGSDNKTYDNKCNFCNAVVDSNGTLTLSHFGKC |
| Ovomucoid-like | SEQ ID NO: 172 | MSSQNQLHRRRRPLPGGQDLNKYYWPHCTSDRFSWLLHVT |
| isoform X1 [Anser | | AEQFRHCVCIYLQPALERPSQEQSTSGQPVDSGSTSTTTM |
| cygnoides domesticus] | | AGIFVLLSLVLCCFPDAAFGVEVDCSRFPNTTNEEGKEVL |
| | | LCTKDLSPICGTDGVTYSNECLLCAYNIEYGTNISKDHDG |
| | | ECKEAVPVDCSTYPNMTNEEGKVMLVCNKMFSPVCGTDGV |
| | | TYDNECMLCAHNVEQGTSVGKKYDGKCKKEVATVDCSDYP |
| | | KPACTVEYMPLCGSDNKTYDNKCNFCNAVVDSNGTLTLSH |
| | | FGKC |
| Ovomucoid [Coturnix | SEQ ID NO: 173 | VEVDCSRFPNTTNEEGKDEVVCPDELRLICGTDGVTYNHE |
| japonica] | | CMLCFYNKEYGTNISKEQDGECGETVPMDCSRYPNTTSED |
| | | GKVTILCTKDFSFVCGTDGVTYDNECMLCAHNVVQGTSVG |
| | | KKHDGECRKELAAVSVDCSEYPKPACPKDYRPVCGSDNKT |
| | | YSNKCNFCNAVVESNGTLTLNHFGKC |
| Ovomucoid [Coturnix | SEQ ID NO: 174 | MAMAGVFLLFSFALCGFLPDAAFGVEVDCSRFPNTTNEEG |
| japonica] | | KDEVVCPDELRLICGTDGVTYNHECMLCFYNKEYGTNISK |
| | | EQDGECGETVPMDCSRYPNTTSEDGKVTILCTKDFSFVCG |
| | | TDGVTYDNECMLCAHNIVQGTSVGKKHDGECRKELAAVSV |
| | | DCSEYPKPACPKDYRPVCGSDNKTYSNKCNFCNAVVESNG |
| | | TLTLNHFGKC |
| Ovomucoid [Anas | SEQ ID NO: 175 | MAGVFVLLSLVLCCFPDAAFGVEVDCSRFPNTTNEEGKDV |
| platyrhynchos] | | LLCTKELSPVCGTDGVTYSNECLLCAYNIEYGTNISKDHD |
| | | GECKEAVPADCSMYPNMTNEEGKMTLLCNKMFSPVCGTDG |
| | | VTYDNECMLCAHNVEQGTSVGKKYDGKCKKEVATVDCSGY |
| | | PKPACTMEYMPLCGSDNKTYGNKCNFCNAVVDSNGTLTLS |
| | | HFGEC |
| Ovomucoid, partial | SEQ ID NO: 176 | QVDCSRFPNTTNEEGKEVLLCTKELSPVCGTDGVTYSNEC |
| [Anas platyrhynchos] | | LLCAYNIEYGTNISKDHDGECKEAVPADCSMYPNMTNEEG |
| | | KMTLLCNKMFSPVCGTDGVTYDNECMLCAHNVEQGTSVGK |
| | | KYDGKCKKEVATVSVDCSGYPKPACTMEYMPLCGSDNKTY |
| | | GNKCNFCNAVV |
| Ovomucoid-like [Tyto | SEQ ID NO: 177 | MTMPGAFVVLSFVLCCFPDATFGVEVDCSTYPNTTNEEGK |
| alba] | | EVLVCSKILSPICGTDGVTYSNECLLCANNIEYGTNISKY |
| | | HDGECKEFVPVNCSRYPNTTNEEGKVMLICNKDLSPVCGT |
| | | DGVTYDNECLLCAHNLEPGTSVGKKYDGECKKEIATVDCS |
| | | DYPKPVCSLESMPLCGSDNKTYSNKCNFCNAVVDSNETLT |
| | | LSHFGKC |
| Ovomucoid [Balearica | SEQ ID NO: 178 | MTMAGVFVLLSFALCCFPDAAFGVEVDCSTYPNTTNEEGK |
| regulorum | | EVLVCTKILSPICGTDGVTYSNECLLCAYNIEYGTNVSKD |
| gibbericeps] | | HDGECKEVVPVDCSRYPNSTNEEGKVVMLCSKDLNPVCGT |
| | | DGVTYDNECVLCAHNVESGTSVGKKYDGECKKETATVDCS |
| | | DYPKPACTLEYMPFCGSDSKTYSNKCNFCNAVVDSNGTLT |
| | | LSHFGKC |
| Turkey vulture | SEQ ID NO: 179 | MTTAGVFVLLSFALCSFPDAAFGVEVDCSTYPNTTNEEGK |
| [Cathartes aura] OVD | | EVLVCTKILSPICGTDGVTYSNECLLCAYNIEYGTNVSKD |
| (native sequence) | | HDGECKEFVPVDCSRYPNTTNEDGKVVLLCNKDLSPICGT |
| bolded is native signal | | DGVTYDNECLLCARNLEPGTSVGKKYDGECKKEIATVDCS |
| sequence | | DYPKPVCSLEYMPLCGSDSKTYSNKCNFCNAVVDSNGTLT |
| | | LSHFGKC |
| Ovomucoid-like | SEQ ID NO: 180 | MTTAGVFVLLSFTLCSFPDAAFGVEVDCSPYPNTTNEEGK |
| [Cuculus canorus] | | EVLVCNKILSPICGTDGVTYSNECLLCAYNLEYGTNISKD |
| | | YDGECKEVAPVDCSRHPNTTNEEGKVELLCNKDLNPICGT |
| | | NGVTYDNECLLCARNLESGTSIGKKYDGECKKEIATVDCS |
| | | DYPKPVCTLEEMPLCGSDNKTYGNKCNFCNAVVDSNGTLT |
| | | LSHFGKC |
| Ovomucoid | SEQ ID NO: 181 | MTTAVVFVLLSFALCCFPDAAFGVEVDCSTYPNSTNEEGK |
| [Antrostomus | | DVLVCPKILGPICGTDGVTYSNECLLCAYNIQYGTNVSKD |
| carolinensis] | | HDGECKEIVPVDCSRYPNTTNEEGKVVFLCNKNFDPVCGT |
| | | DGDTYDNECMLCARSLEPGTTVGKKHDGECKREIATVDCS |
| | | DYPKPTCSAEDMPLCGSDSKTYSNKCNFCNAVVDSNGTLT |
| | | LSRFGKC |
| Ovomucoid [Cariama | SEQ ID NO: 182 | MTMTGVFVLLSFAICCFPDAAFGVEVDCSTYPNTTNEEGK |
| cristata] | | EVLVCTKILSPICGTDGVTYSNECLLCAYNIEYGTNVSKD |
| | | HDGECKEVVPVDCSKYPNTTNEEGKVVLLCSKDLSPVCGT |
| | | DGVTYDNECLLCARNLEPGSSVGKKYDGECKKEIATIDCS |
| | | DYPKPVCSLEYMPLCGSDSKTYDNKCNFCNAVVDSNGTLT |
| | | LSHFGKC |
| Ovomucoid-like | SEQ ID NO: 183 | MTTAGVFVLLSFVLCCFPDAVFGVEVDCSTYPNTTNEEGK |
| isoform X2 | | EVLVCTKILSPICGTDGVTYSNECLLCAYNIEYGTNVSKD |
| [Pygoscelis adeliae] | | HDGECKEVVPVNCSRYPNTTNEEGKVVLRCSKDLSPVCGT |
| | | DGVTYDNECLMCARNLEPGAVVGKNYDGECKKEIATVDCS |
| | | DYPKPVCSLEYMPLCGSDSKTYSNKCNFCNAVVDSNGTLT |
| | | LSHFGKC |
| Ovomucoid-like | SEQ ID NO: 184 | MTTAGVFVLLSIALCCFPDAAFGVEVDCSAYSNTTSEEGK |
| [Nipponia nippon] | | EVLSCTKILSPICGTDGVTYSNECLLCAYNIEYGTNISKD |
| | | HDGECKEVVSVDCSRYPNTTNEEGKAVLLCNKDLSPVCGT |
| | | DGVTYDNECLLCAHNLEPGTSVGKKYDGACKKEIATVDCS |
| | | DYPKPVCTLEYLPLCGSDSKTYSNKCDFCNAVVDSNGTLT |
| | | LSHFGKC |
| Ovomucoid-like | SEQ ID NO: 185 | MTTAGVFVLLSFALCCFPDAAFGVEVDCSTYPNTTNEEGK |
| [Phaethon lepturus] | | EVLVCTKILSPICGTDGTTYSNECLLCAYNIEYGTNVSKD |
| | | HDGECKVVPVDCSKYPNTTNEDGKVVLLCNKALSPICGTD |
| | | RVTYDNECLMCAHNLEPGTSVGKKHDGECQKEVATVDCSD |
| | | YPKPVCSLEYMPLCGSDGKTYSNKCNFCNAVVNSNGTLTL |
| | | SHFEKC |
| Ovomucoid-like | SEQ ID NO: 186 | MTTAGVFVLLSFVLCCFFPDAAFGVEVDCSTYPNTTNEEG |
| isoform X1 | | KEVLVCAKILSPVCGTDGVTYSNECLLCAHNIENGTNVGK |
| [Melopsittacus | | DHDGKCKEAVPVDCSRYPNTTDEEGKVVLLCNKDVSPVCG |
| undulatus] | | TDGVTYDNECLLCAHNLEAGTSVDKKNDSECKTEDTTLAA |
| | | VSVDCSDYPKPVCTLEYLPLCGSDNKTYSNKCRFCNAVVD |
| | | SNGTLTLSRFGKC |
| Ovomucoid [Podiceps | SEQ ID NO: 187 | MTTAGVFVLLSFALCCSPDAAFGVEVDCSTYPNTTNEEGK |
| cristatus] | | EVLACTKILSPICGTDGVTYSNECLLCAYNMEYGTNVSKD |
| | | HDGKCKEVVPVDCSRYPNTTNEEGK |
| | | VVLLCNKDLSPVCGTDGVTYDNECLLCARNLEPGASVGKK |
| | | YDGECKKEIATVDCSDYPKPVCSLEHMPLCGSDSKTYSNK |
| | | CTFCNAVVDSNGTLTLSHFGKC |
| Ovomucoid-like | SEQ ID NO: 188 | MTTAGVFVLLSFALCCFPDAAFGVEVDCSTYPNTTNEEGR |
| [Fulmarus glacialis] | | EVLVCTKILSPICGTDGVTYSNECLLCAYNIEYGTNVSKD |
| | | HDGECKEVAPVGCSRYPNTTNEEGKVVLLCNKDLSPVCGT |
| | | DGVTYDNECLLCARHLEPGTSVGKKYDGECKKEIATVDCS |
| | | DYPKPVCSLEYMPLCGSDSKTYSNKCNFCNAVLDSNGTLT |
| | | LSHFGKC |
| Ovomucoid | SEQ ID NO: 189 | MTTAGVFVLLSFALCCFPDAVFGVEVDCSTYPNTTNEEGK |
| [Aptenodytes forsteri] | | EVLVCTKILSPICGTDGVTYSNECLLCAYNIEYGTNVSKD |
| | | HDGECKEVVPVDCSRYPNTTNEEGKVVLRCNKDLSPVCGT |
| | | DGVTYDNECLMCARNLEPGAIVGKKYDGECKKEIATVDCS |
| | | DYPKPVCSLEYMPLCGSDSKTYSNKCNFCNAVVDSNGTLI |
| | | LSHFGKC |
| Ovomucoid-like | SEQ ID NO: 190 | MTTAGVFVLLSFVLCCFPDAVFGVEVDCSTYPNTTNEEGK |
| isoform X1 | | EVLVCTKILSPICGTDGVTYSNECLLCAYNIEYGTNVSKD |
| [Pygoscelis adeliae] | | HDGECKEVVPVDCSRYPNTTNEEGKVVLRCSKDLSPVCGT |
| | | DGVTYDNECLMCARNLEPGAVVGKNYDGECKKEIATVDCS |
| | | DYPKPVCSLEYMPLCGSDSKTYSNKCNFCNAVVDSNGTLT |
| | | LSHFGKC |
| Ovomucoid isoform | SEQ ID NO: 191 | MSSQNQLPSRCRPLPGSQDLNKYYQPHCTGDRFCWLFYVT |
| X1 [Aptenodytes | | VEQFRHCICIYLQLALERPSHEQSGQPADSRNTSTMTTAG |
| forsteri] | | VFVLLSFALCCFPDAVFGVEVDCSTYPNTTNEEGKEVLVC |
| | | TKILSPICGTDGVTYSNECLLCAYNIEYGTNVSKDHDGEC |
| | | KEVVPVDCSRYPNTTNEEGKVVLRCNKDLSPVCGTDGVTY |
| | | DNECLMCARNLEPGAIVGKKYDGECKKEIATVDCSDYPKP |
| | | VCSLEYMPLCGSDSKTYSNKCNFCNAVVDSNGTLILSHFG |
| | | KC |
| Ovomucoid, partial | SEQ ID NO: 192 | MTTAVVFVLLSFALCCFPDAAFGVEVDCSTYPNSTNEEGK |
| [Antrostomus | | DVLVCPKILGPICGTDGVTYSNECLLCAYNIQYGTNVSKD |
| carolinensis] | | HDGECKEIVPVDCSRYPNTTNEEGKVVFLCNKNFDPVCGT |
| | | DGDTYDNECMLCARSLEPGTTVGKKHDGECKREIATVDCS |
| | | DYPKPTCSAEDMPLCGSDSKTYSNKCNFCNAVV |
| rOVD as expressed in | SEQ ID NO: 193 | EAEAAEVDCSRFPNATDKEGKDVLVCNKDLRPICGTDGVT |
| pichia | | YTNDCLLCAYSIEFGTNISKEHDGECKETVPMNCSSYANT |
| secreted form 1 | | TSEDGKVMVLCNRAFNPVCGTDGVTYDNECLLCAHKVEQG |
| | | ASVDKRHDGGCRKELAAVSVDCSEYPKPDCTAEDRPLCGS |
| | | DNKTYGNKCNFCNAVVESNGTLTLSHFGKC |
| rOVD as expressed in | SEQ ID NO: 194 | EEGVSLEKREAEAAEVDCSRFPNATDKEGKDVLVCNKDLR |
| pichia secreted form 2 | | PICGTDGVTYTNDCLLCAYSIEFGTNISKEHDGECKETVP |
| | | MNCSSYANTTSEDGKVMVLCNRAFNPVCGTDGVTYDNECL |
| | | LCAHKVEQGASVDKRHDGGCRKELAAVSVDCSEYPKPDCT |
| | | AEDRPLCGSDNKTYGNKCNFCNAVVESNGTLTLSHFGKC |
| rOVD [gallus] coding | SEQ ID NO: 195 | MRFPSIFTAVLFAASSALAAPVNTTTEDETAQIPAEAVIG |
| sequence containing | | YSDLEGDFDVAVLPFSNSTNNGLLFINTTIASIAAKEEGV |
| an alpha mating factor | | SLEKREAEAAEVDCSRFPNATDKEGKDVLVCNKDLRPICG |
| signal sequence | | TDGVTYTNDCLLCAYSIEFGTNISKEHDGECKETVPMNCS |
| (bolded) as expressed | | SYANTTSEDGKVMVLCNRAFNPVCGTDGVTYDNECLLCAH |
| in pichia | | KVEQGASVDKRHDGGCRKELAAVSVDCSEYPKPDCTAEDR |
| | | PLCGSDNKTYGNKCNFCNAVVESNGTLTLSHFGKC |
| Turkey vulture OVD | SEQ ID NO: 196 | MRFPSIFTAVLFAASSALAAPVNTTTEDETAQIPAEAVIG |
| coding sequence | | YSDLEGDFDVAVLPFSNSTNNGLLFINTTIASIAAKEEGV |
| containing secretion | | SLEKREAEAVEVDCSTYPNTTNEEGKEV |
| signals as expressed in | | LVCTKILSPICGTDGVTYSNECLLCAYNIEYGTNVSKDHD |
| pichia | | GECKEFVPVDCSRYPNTTNEDGKVVLLCNKDLSPICGTDG |
| bolded is an alpha | | VTYDNECLLCARNLEPGTSVGKKYDGECKKEIATVDCSDY |
| mating factor signal | | PKPVCSLEYMPLCGSDSKTYSNKCNFCNAVVDSNGTLTLS |
| sequence | | HFGKC |
| Turkey vulture OVD | SEQ ID NO: 197 | EAEAVEVDCSTYPNTTNEEGKEVLVCTKILSPICGTDGVT |
| in secreted form | | YSNECLLCAYNIEYGTNVSKDHDGECKEFVPVDCSRYPNT |
| expressed in Pichia | | TNEDGKVVLLCNKDLSPICGTDGVTYDNECLLCARNLEPG |
| | | TSVGKKYDGECKKEIATVDCSDYPKPVCSLEYMPLCGSDS |
| | | KTYSNKCNFCNAVVDSNGTLTLSHFGKC |
| Humming bird | SEQ ID NO: 198 | MTMAGVFVLLSFILCCFPDTAFGVEVDCSIYPNTTSEEGK |
| OVD (native | | EVLVCTETLSPICGSDGVTYNNECQLCAYNVEYGTNVSKD |
| sequence) | | HDGECKEIVPVDCSRYPNTTEEGRVVMLCNKALSPVCGTD |
| bolded is the native | | GVTYDNECLLCARNLESGTSVGKKFDGECKKEIATVDCTD |
| signal sequence | | YPKPVCSLDYMPLCGSDSKTYSNKCNFCNAVMDSNGTLTL |
| | | NHFGKC |
| Humming bird OVD | SEQ ID NO: 199 | MRFPSIFTAVLFAASSALAAPVNTTTEDETAQIPAEAVIG |
| coding sequence as | | YSDLEGDFDVAVLPFSNSTNNGLLFINTTIASIAAKEEGV |
| expressed in Pichia | | SLDKREAEAVEVDCSIYPNTTSEEGKEVLVCTETLSPICG |
| bolded is an alpha | | SDGVTYNNECQLCAYNVEYGTNVSKDHDGECKEIVPVDCS |
| mating factor signal | | RYPNTTEEGRVVMLCNKALSPVCGTDGVTYDNECLLCARN |
| sequence | | LESGTSVGKKFDGECKKEIATVDCTDYPKPVCSLDYMPLC |
| | | GSDSKTYSNKCNFCNAVMDSNGTLTLNHFGKC |
| Humming bird OVD | SEQ ID NO: 200 | EAEAVEVDCSIYPNTTSEEGKEVLVCTETLSPICGSDGVT |
| in secreted form from | | YNNECQLCAYNVEYGTNVSKDHDGECKEIVPVDCSRYPNT |
| Pichia | | TEEGRVVMLCNKALSPVCGTDGVTYDNECLLCARNLESGT |
| | | SVGKKFDGECKKEIATVDCTDYPKPVCSLDYMPLCGSDSK |
| | | TYSNKCNFCNAVMDSNGTLTLNHFGKC |
| Ovalbumin related | SEQ ID NO: 201 | MFFYNTDFRMGSISAANAEFCFDVFNELKVQHTNENILYS |
| protein X | | PLSIIVALAMVYMGARGNTEYQMEKALHFDSIAGLGGSTQ |
| | | TKVQKPKCGKSVNIHLLFKELLSDITASKANYSLRIANRL |
| | | YAEKSRPILPIYLKCVKKLYRAGLETVNFKTASDQARQLI |
| | | NSWVEKQTEGQIKDLLVSSSTDLDTTLVLVNAIYFKGMWK |
| | | TAFNAEDTREMPFHVTKEESKPVQMMCMNNSFNVATLPAE |
| | | KMKILELPFASGDLSMLVLLPDEVSGLERIEKTINFEKLT |
| | | EWTNPNTMEKRRVKVYLPQMKIEEKYNLTSVLMALGMTDL |
| | | FIPSANLTGISSAESLKISQAVHGAFMELSEDGIEMAGST |
| | | GVIEDIKHSPELEQFRADHPFLFLIKHNPTNTIVYFGRYW |
| | | SP* |
| Ovalbumin related | SEQ ID NO: 202 | MDSISVTNAKFCFDVFNEMKVHHVNENILYCPLSILTALA |
| protein Y | | MVYLGARGNTESQMKKVLHFDSITGAGSTTDSQCGSSEYV |
| | | HNLFKELLSEITRPNATYSLEIADKLYVDKTFSVLPEYLS |
| | | CARKFYTGGVEEVNFKTAAEEARQLINSWVEKETNGQIKD |
| | | LLVSSSIDFGTTMVFINTIYFKGIWKIAFNTEDTREMPFS |
| | | MTKEESKPVQMMCMNNSFNVATLPAEKMKILELPYASGDL |
| | | SMLVLLPDEVSGLERIEKTINFDKLREWTSTNAMAKKSMK |
| | | VYLPRMKIEEKYNLTSILMALGMTDLFSRSANLTGIS |
| | | SVDNLMISDAVHGVFMEVNEEGTEATGSTGAIGNIKHSLE |
| | | LEEFRADHPFLFFIRYNPTNAILFFGRYWSP* |
| Ovalbumin | SEQ ID NO: 203 | MGSIGAASMEFCFDVFKELKVHHANENIFYCPIAIMSALA |
| | | MVYLGAKDSTRTQINKVVRFDKLPGFGDSIEAQCGTSVNV |
| | | HSSLRDILNQITKPNDVYSFSLASRLYAEERYPILPEYLQ |
| | | CVKELYRGGLEPINFQTAADQARELINSWVESQINGIIRN |
| | | VLQPSSVDSQTAMVLVNAIVFKGLWEKAFKDEDTQAMPFR |
| | | VTEQESKPVQMMYQIGLFRVASMASEKMKILELPFASGTM |
| | | SMLVLLPDEVSGLEQLESIINFEKLTEWTSSNVMEERKIK |
| | | VYLPRMKMEEKYNLTSVLMAMGITDVFSSSANLSGISSAE |
| | | SLKISQAVHAAHAEINEAGREVVGSAEAGVDAASVSEEFR |
| | | ADHPFLFCIKHIATNAVLFFGRCVSP* |
| Chicken Ovalbumin | SEQ ID NO: 204 | MRFPSIFTAVLFAASSALAAPVNTTTEDETAQIPAEAVIG |
| with bolded signal | | YSDLEGDFDVAVLPFSNSTNNGLLFINTTIASIAAKEEGV |
| sequence | | SLDKREAEAGSIGAASMEFCFDVFKELKVHHANENIFYCP |
| | | IAIMSALAMVYLGAKDSTRTQINKVVRFDKLPGFGDSIEA |
| | | QCGTSVNVHSSLRDILNQITKPNDVYSFSLASRLYAEERY |
| | | PILPEYLQCVKELYRGGLEPINFQTAADQARELINSWVES |
| | | QTNGIIRNVLQPSSVDSQTAMVLVNAIVFKGLWEKAFKDE |
| | | DTQAMPFRVTEQESKPVQMMYQIGLFRVASMASEKMKILE |
| | | LPFASGTMSMLVLLPDEVSGLEQLESIINFEKLTEWTSSN |
| | | VMEERKIKVYLPRMKMEEKYNLTSVLMAMGITDVFSSSAN |
| | | LSGISSAESLKISQAVHAAHAEINEAGREVVGSAEAGVDA |
| | | ASVSEEFRADHPFLFCIKHIATNAVLFFGRCVSP |
| Chicken OVA | SEQ ID NO: 205 | EAEAGSIGAASMEFCFDVFKELKVHHANENIFYCPIAIMS |
| sequence as secreted | | ALAMVYLGAKDSTRTQINKVVRFDKLPGFGDSIEAQCGTS |
| from pichia | | VNVHSSLRDILNQITKPNDVYSFSLASRLYAEERYPILPE |
| | | YLQCVKELYRGGLEPINFQTAADQARELINSWVESQTNGI |
| | | IRNVLQPSSVDSQTAMVLVNAIVFKGLWEKAFKDEDTQAM |
| | | PFRVTEQESKPVQMMYQIGLFRVASMASEKMKILELPFAS |
| | | GTMSMLVLLPDEVSGLEQLESIINFEKLTEWTSSNVMEER |
| | | KIKVYLPRMKMEEKYNLTSVLMAMGITDVFSSSANLSGIS |
| | | SAESLKISQAVHAAHAEINEAGREVVGSAEAGVDAASVSE |
| | | EFRADHPFLFCIKHIATNAVLFFGRCVSP |
| Predicted Ovalbumin | SEQ ID NO: 206 | MRVPAQLLGLLLLWLPGARCGSIGAASMEFCFDVFKELKV |
| [Achromobacter | | HHANENIFYCPIAIMSALAMVYLGAKDSTRTQINKVVRFD |
| denitrificans] | | KLPGFGDSIEAQCGTSVNVHSSLRDILNQITKPNDVYSFS |
| | | LASRLYAEERYPILPEYLQCVKELYRGGLEPINFQTAADQ |
| | | ARELINSWVESQTNGIIRNVLQPSSVDSQTAMVLVNAIVF |
| | | KGLWEKAFKDEDTQAMPFRVTEQESKPVQMMYQIGLFRVA |
| | | SMASEKMKILELPFASGTMSMLVLLPDEVSGLEQLESIIN |
| | | FEKLTEWTSSNVMEERKIKVYLPRMKMEEKYNLTSVLMAM |
| | | GITDVFSSSANLSGISSAESLKISQAVHAAHAEINEAGRE |
| | | VVGSAEAGVDAASVSEEFRADHPFLFCIKHIATNAVLFFG |
| | | RCVSPLEIKRAAAHHHHHH |
| OLLAS epitope- | SEQ ID NO: 207 | MTSGFANELGPRLMGKLTMGSIGAASMEFCFDVFKELKVH |
| tagged ovalbumin | | HANENIFYCPIAIMSALAMVYLGAKDSTRTQINKVVRFDK |
| | | LPGFGDSIEAQCGTSVNVHSSLRDILNQITKPNDVYSFSL |
| | | ASRLYAEERYPILPEYLQCVKELYRGGLEPINFQTAADQA |
| | | RELINSWVESQTNGIIRNVLQPSSVDSQTAMVLVNAIVFK |
| | | GLWEKTFKDEDTQAMPFRVTEQESKPVQMMYQIGLFRVAS |
| | | MASEKMKILELPFASGTMSMLVLLP |
| | | DEVSGLEQLESIINFEKLTEWTSSNVMEERKIKVYLPRMK |
| | | MEEKYNLTSVLMAMGITDVFSSSANLSGISSAESLKISQA |
| | | VHAAHAEINEAGREVVGSAEAGVDAASVSEEFRADHPFLF |
| | | CIKHIATNAVLFFGRCVSPSR |
| Serpin family protein | SEQ ID NO: 208 | MGGRRVRWEVYISRAGYVNRQIAWRRHHRSLTMRVPAQLL |
| [Achromobacter | | GLLLLWLPGARCGSIGAASMEFCFDVFKELKVHHANENIF |
| denitrificans] | | YCPIAIMSALAMVYLGAKDSTRTQINKVVRFDKLPGFGDS |
| | | IEAQCGTSVNVHSSLRDILNQITKPNDVYSFSLASRLYAE |
| | | ERYPILPEYLQCVKELYRGGLEPINFQTAADQARELINSW |
| | | VESQTNGIIRNVLQPSSVDSQTAMVLVNAIVFKGLWEKAF |
| | | KDEDTQAMPFRVTEQESKPVQMMYQIGLFRVASMASEKMK |
| | | ILELPFASGTMSMLVLLPDEVSGLEQLESIINFEKLTEWT |
| | | SSNVMEERKIKVYLPRMKMEEKYNLTSVLMAMGITDVFSS |
| | | SANLSGISSAESLKISQAVHAAHAEINEAGREVVGSAEAG |
| | | VDAASVSEEFRADHPFLFCIKHIATNAVLFFGRCVSPLEI |
| | | KRAAAHHHHHH |
| PREDICTED: | SEQ ID NO: 209 | MGSIGAVSMEFCFDVFKELKVHHANENIFYSPFTIISALA |
| ovalbumin isoform X1 | | MVYLGAKDSTRTQINKVVRFDKLPGFGDSVEAQCGTSVNV |
| [Meleagris gallopavo] | | HSSLRDILNQITKPNDVYSFSLASRLYAEETYPILPEYLQ |
| | | CVKELYRGGLESINFQTAADQARGLINSWVESQTNGMIKN |
| | | VLQPSSVDSQTAMVLVNAIVFKGLWEKAFKDEDTQAIPFR |
| | | VTEQESKPVQMMYQIGLFKVASMASEKMKILELPFASGTM |
| | | SMWVLLPDEVSGLEQLETTISFEKMTEWISSNIMEERRIK |
| | | VYLPRMKMEEKYNLTSVLMAMGITDLFSSSANLSGISSAG |
| | | SLKISQAVHAAYAEIYEAGREVIGSAEAGADATSVSEEFR |
| | | VDHPFLYCIKHNLTNSILFFGRCISP |
| Ovalbumin precursor | SEQ ID NO: 210 | MGSIGAVSMEFCFDVFKELKVHHANENIFYSPFTIISALA |
| [Meleagris gallopavo] | | MVYLGAKDSTRTQINKVVRFDKLPGFGDSVEAQCGTSVNV |
| | | HSSLRDILNQITKPNDVYSFSLASRLYAEETYPILPEYLQ |
| | | CVKELYRGGLESINFQTAADQARGLINSWVESQTNGMIKN |
| | | VLQPSSVDSQTAMVLVNAIVFKGLWEKAFKDEDTQAIPFR |
| | | VTEQESKPVQMMYQIGLFKVASMASEKMKILELPFASGTM |
| | | SMWVLLPDEVSGLEQLETTISFEKMTEWISSNIMEERRIK |
| | | VYLPRMKMEEKYNLTSVLMAMGITDLFSSSANLSGISSAG |
| | | SLKISQAAHAAYAEIYEAGREVIGSAEAGADATSVSEEFR |
| | | VDHPFLYCIKHNLTNSILFFGRCISP |
| Hypothetical protein | SEQ ID NO: 211 | YYRVPCMVLCTAFHPYIFIVLLFALDNSEFTMGSIGAVSM |
| [Bambusicola | | EFCFDVFKELRVHHPNENIFFCPFAIMSAMAMVYLGAKDS |
| thoracicus] | | TRTQINKVIRFDKLPGFGDSTEAQCGKSANVHSSLKDILN |
| | | QITKPNDVYSFSLASRLYADETYSIQSEYLQCVNELYRGG |
| | | LESINFQTAADQARELINSWVESQTNGIIRNVLQPSSVDS |
| | | QTAMVLVNAIVFRGLWEKAFKDEDTQTMPFRVTEQESKPV |
| | | QMMYQIGSFKVASMASEKMKILELPLASGTMSMLVLLPDE |
| | | VSGLEQLETTISFEKLTEWTSSNVMEERKIKVYLPRMKME |
| | | EKYNLTSVLMAMGITDLFRSSANLSGISLAGNLKISQAVH |
| | | AAHAEINEAGRKAVSSAEAGVDATSVSEEFRADRPFLFCI |
| | | KHIATKVVFFFGRYTSP |
| Egg albumin | SEQ ID NO: 212 | MGSIGAASMEFCFDVFKELKVHHANDNMLYSPFAILSTLA |
| | | MVFLGAKDSTRTQINKVVHFDKLPGFGDSIEAQCGTSVNV |
| | | HSSLRDILNQITKQNDAYSFSLASRLYAQETYTVVPEYLQ |
| | | CVKELYRGGLESVNFQTAADQARGLINAWVESQTNGIIRN |
| | | ILQPSSVDSQTAMVLVNAIAFKGLWEKAFKAEDTQTIPFR |
| | | VTEQESKPVQM |
| | | MYQIGSFKVASMASEKMKILELPFASGTMSMLVLLPDDVS |
| | | GLEQLESIISFEKLTEWTSSSIMEERKVKVYLPRMKMEEK |
| | | YNLTSLLMAMGITDLFSSSANLSGISSVGSLKISQAVHAA |
| | | HAEINEAGRDVVGSAEAGVDATEEFRADHPFLFCVKHIET |
| | | NAILLFGRCVSP |
| Ovalbumin isoform | SEQ ID NO: 213 | MASIGAVSTEFCVDVYKELRVHHANENIFYSPFTIISTLA |
| X2 [Numida | | MVYLGAKDSTRTQINKVVRFDKLPGFGDSIEAQCGTSVNV |
| meleagris] | | HSSLRDILNQITKPNDVYSFSLASRLYAEETYPILPEYLQ |
| | | CVKELYRGGLESINFQTAADQARELINSWVESQTSGIIKN |
| | | VLQPSSVNSQTAMVLVNAIYFKGLWERAFKDEDTQAIPFR |
| | | VTEQESKPVQMMSQIGSFKVASVASEKVKILELPFVSGTM |
| | | SMLVLLPDEVSGLEQLESTISTEKLTEWTSSSIMEERKIK |
| | | VFLPRMRMEEKYNLTSVLMAMGMTDLFSSSANLSGISSAE |
| | | SLKISQAVHAAYAEIYEAGREVVSSAEAGVDATSVSEEFR |
| | | VDHPFLLCIKHNPTNSILFFGRCISP |
| Ovalbumin isoform | SEQ ID NO: 214 | MALCKAFHPYIFIVLLFDVDNSAFTMASIGAVSTEFCVDV |
| X1 [Numida | | YKELRVHHANENIFYSPFTIISTLAMVYLGAKDSTRTQIN |
| meleagris] | | KVVRFDKLPGFGDSIEAQCGTSVNVHSSLRDILNQITKPN |
| | | DVYSFSLASRLYAEETYPILPEYLQCVKELYRGGLESINF |
| | | QTAADQARELINSWVESQTSGIIKNVLQPSSVNSQTAMVL |
| | | VNAIYFKGLWERAFKDEDTQAIPFRVTEQESKPVQMMSQI |
| | | GSFKVASVASEKVKILELPFVSGTMSMLVLLPDEVSGLEQ |
| | | LESTISTEKLTEWTSSSIMEERKIKVFLPRMRMEEKYNLT |
| | | SVLMAMGMTDLFSSSANLSGISSAESLKISQAVHAAYAEI |
| | | YEAGREVVSSAEAGVDATSVSEEFRVDHPFLLCIKHNPTN |
| | | SILFFGRCISP |
| PREDICTED: | SEQ ID NO: 215 | MGSIGAASMEFCFDVFKELKVHHANDNMLYSPFAILSTLA |
| Ovalbumin isoform | | MVFLGAKDSTRTQINKVVHFDKLPGFGDSIEAQCGTSANV |
| X2 [Coturnix | | HSSLRDILNQITKQNDAYSFSLASRLYAQETYTVVPEYLQ |
| japonica] | | CVKELYRGGLESVNFQTAADQARGLINAWVESQTNGIIRN |
| | | ILQPSSVDSQTAMVLVNAIAFKGLWEKAFKAEDTQTIPFR |
| | | VTEQESKPVQMMHQIGSFKVASMASEKMKILELPFASGTM |
| | | SMLVLLPDDVSGLEQLESTISFEKLTEWTSSSIMEERKVK |
| | | VYLPRMKMEEKYNLTSLLMAMGITDLFSSSANLSGISSVG |
| | | SLKISQAVHAAYAEINEAGRDVVGSAEAGVDATEEFRADH |
| | | PFLFCVKHIETNAILLFGRCVSP |
| PREDICTED: | SEQ ID NO: 216 | MGLCTAFHPYIFIVLLFALDNSEFTMGSIGAASMEFCFDV |
| ovalbumin isoform X1 | | FKELKVHHANDNMLYSPFAILSTLAMVFLGAKDSTRTQIN |
| [Coturnix japonica] | | KVVHFDKLPGFGDSIEAQCGTSANVHSSLRDILNQITKQN |
| | | DAYSFSLASRLYAQETYTVVPEYLQCVKELYRGGLESVNF |
| | | QTAADQARGLINAWVESQTNGIIRNILQPSSVDSQTAMVL |
| | | VNAIAFKGLWEKAFKAEDTQTIPFRVTEQESKPVQMMHQI |
| | | GSFKVASMASEKMKILELPFASGTMSMLVLLPDDVSGLEQ |
| | | LESTISFEKLTEWTSSSIMEERKVKVYLPRMKMEEKYNLT |
| | | SLLMAMGITDLFSSSANLSGISSVGSLKISQAVHAAYAEI |
| | | NEAGRDVVGSAEAGVDATEEFRADHPFLFCVKHIETNAIL |
| | | LFGRCVSP |
| Egg albumin | SEQ ID NO: 217 | MGSIGAASMEFCFDVFKELKVHHANDNMLYSPFAILSTLA |
| | | MVFLGAKDSTRTQINKVVHFDKLPGFGDSIEAQCGTSANV |
| | | HSSLRDILNQITKQNDAYSFSLASRLYAQETYTVVPEYLQ |
| | | CVKELYRGGLESVNFQTAADQARGLINAWVESQINGIIRN |
| | | ILQPSSVDSQTAMVLVNAIAFKGLWEKAFKAEDTQTIPFR |
| | | VTEQESKPVQM |
| | | MHQIGSFKVASMASEKMKILELPFASGTMSMLVLLPDDVS |
| | | GLEQLESTISFEKLTEWTSSSIMEERKVKVYLPRMKMEEK |
| | | YNLTSLLMAMGITDLFSSSANLSGISSVGSLKIPQAVHAA |
| | | YAEINEAGRDVVGSAEAGVDATEEFRADHPFLFCVKHIET |
| | | NAILLFGRCVSP |
| ovalbumin [Anas | SEQ ID NO: 218 | MGSIGAASTEFCFDVFRELRVQHVNENIFYSPFSIISALA |
| platyrhynchos] | | MVYLGARDNTRTQIDKVVHFDKLPGFGESMEAQCGTSVSV |
| | | HSSLRDILTQITKPSDNFSLSFASRLYAEETYAILPEYLQ |
| | | CVKELYKGGLESISFQTAADQARELINSWVESQTNGIIKN |
| | | ILQPSSVDSQTTMVLVNAIYFKGMWEKAFKDEDTQAMPFR |
| | | MTEQESKPVQMMYQVGSFKVAMVTSEKMKILELPFASGMM |
| | | SMFVLLPDEVSGLEQLESTISFEKLTEWTSSTMMEERRMK |
| | | VYLPRMKMEEKYNLTSVFMALGMTDLFSSSANMSGISSTV |
| | | SLKMSEAVHAACVEIFEAGRDVVGSAEAGMDVTSVSEEFR |
| | | ADHPFLFFIKHNPTNSILFFGRWMSP |
| PREDICTED: | SEQ ID NO: 219 | MGSIGAASTEFCFDVFRELKVQHVNENIFYSPLSIISALA |
| ovalbumin-like [Anser | | MVYLGARDNTRTQIDQVVHFDKIPGFGESMEAQCGTSVSV |
| cygnoides domesticus] | | HSSLRDILTEITKPSDNFSLSFASRLYAEETYTILPEYLQ |
| | | CVKELYKGGLESISFQTAADQARELINSWVESQTNGIIKN |
| | | ILQPSSVDSQTTMVLVNAIYFKGMWEKAFKDEDTQTMPFR |
| | | MTEQESKPVQMMYQVGSFKLATVTSEKVKILELPFASGMM |
| | | SMCVLLPDEVSGLEQLETTISFEKLTEWTSSTMMEERRMK |
| | | VYLPRMKMEEKYNLTSVFMALGMTDLFSSSANMSGISSTV |
| | | SLKMSEAVHAACVEIFEAGRDVVGSAEAGMDVTSVSEEFR |
| | | ADHPFLFFIKHNPSNSILFFGRWISP |
| PREDICTED: | SEQ ID NO: 220 | MGSIGAASTEFCFDVFKELKVQHVNENIFYSPLTIISALS |
| Ovalbumin-like | | MVYLGARENTRAQIDKVLHFDKMPGFGDTIESQCGTSVSI |
| [Aquila chrysaetos | | HTSLKDMFTQITKPSDNYSLSFASRLYAEETYPILPEYLQ |
| canadensis] | | CVKELYKGGLETISFQTAAEQARELINSWVESQTNGMIKN |
| | | ILQPSSVDPQTKMVLVNAIYFKGVWEKAFKDEDTQEVPFR |
| | | VTEQESKPVQMMYQIGSFKVAVMASEKMKILELPYASGQL |
| | | SMLVLLPDDVSGLEQLESAITFEKLMAWTSSTTMEERKMK |
| | | VYLPRMKIEEKYNLTSVLMALGVTDLFSSSANLSGISSAE |
| | | SLKISKAVHEAFVEIYEAGSEVVGSTEAGMEVTSVSEEFR |
| | | ADHPFLFLIKHNPTNSILFFGRCFSP |
| PREDICTED: | SEQ ID NO: 221 | MGSIGAASTEFCFDVFKELKVQHVNENIFYSPLTIISALS |
| Ovalbumin-like | | MVYLGARENTRTQIDKVLHFDKMTGFGDTVESQCGTSVSI |
| [Haliaeetus albicilla] | | HTSLKDIFTQITKPSDNYSLSLASRLYAEETYPILPEYLQ |
| | | CVKELYKGGLETVSFQTAAEQARELINSWVESQTNGMIKN |
| | | ILQPSSVDPQTKMVLVNAIYFKGVWEKAFKDEDTQEVPFR |
| | | VTEQESKPVQMMYQIGSFKVAVMASEKMKILELPYASGQL |
| | | SMLVLLPDDVSGLEQLESAITSEKLMEWTSSTTMEERKMK |
| | | VYLPRMKIEEKYNLTSVLMALGVTDLFSSSADLSGISSAE |
| | | SLKISKAVHEAFVEIYEAGSEVVGSTEGGMEVTSVSEEFR |
| | | ADHPFLFLIKHKPTNSILFFGRCFSP |
| PREDICTED: | SEQ ID NO: 222 | MGSIGAASTEFCFDVFKELKVQHVNENIFYSPLTIISALS |
| Ovalbumin-like | | MVYLGARENTRTQIDKVLHFDKMTGFGDTVESQCGTSVSI |
| [Haliaeetus | | HTSLKDIFTQITKPSDNYSLSLASRLYAEETYPILPEYLQ |
| leucocephalus] | | CVKELYKGGLETVSFQTAAEQARELINSWVESQTNGMIKN |
| | | ILQPSSVDPQTKMVLVNAIYFKGVWEKAFKDEDTQEVPFR |
| | | VTEQESKPVQMMY |
| | | QIGSFKVAVMASEKMKILELPYASGQLSMLVLLPDDVSGL |
| | | EQLESAITSEKLMEWTSSTTMEERKMKVYLPRMKIEEKYN |
| | | LTSVLMALGVTDLFSSSADLSGISSAESLKISKAVHEAFV |
| | | EIYEAGSEVVGSTEGGMEVTSFSEEFRADHPFLFLIKHKP |
| | | TNSILFFGRCFSP |
| PREDICTED: | SEQ ID NO: 223 | MGSIGAASTEFCFDVFKELKVQHVNENIFYSPLSIISALS |
| Ovalbumin [Fulmarus | | MVYLGARENTRAQIDKVVHFDKITGFGETIESQCGTSVSV |
| glacialis] | | HTSLKDMFTQITKPSDNYSLSFASRLYAEETYPILPEYLQ |
| | | CVKELYKGGLETTSFQTAADQARELINSWVESQTNGMIKN |
| | | ILQPGSVDPQTEMVLVNAIYFKGMWEKAFKDEDTQAVPFR |
| | | MTEQESKTVQMMYQIGSFKVAVMASEKMKILELPYASGEL |
| | | SMLVMLPDDVSGLEQLETAITFEKLMEWTSSNMMEERKMK |
| | | VYLPRMKMEEKYNLTSVLMALGVTDLFSSSANLSGISSAE |
| | | SLKMSEAVHEAFVEIYEAGSEVVGSTGAGMEVTSVSEEFR |
| | | ADHPFLFLIKHNPTNSILFFGRCFSP |
| PREDICTED: | SEQ ID NO: 224 | MGSIGAASTEFCFDVFKELRVQHVNENVCYSPLIIISALS |
| Ovalbumin-like | | LVYLGARENTRAQIDKVVHFDKITGFGESIESQCGTSVSV |
| [Chlamydotis | | HTSLKDMFNQITKPSDNYSLSVASRLYAEERYPILPEYLQ |
| macqueenii] | | CVKELYKGGLESISFQTAADQAREAINSWVESQTNGMIKN |
| | | ILQPSSVDPQTEMVLVNAIYFKGMWQKAFKDEDTQAVPFR |
| | | ISEQESKPVQMMYQIGSFKVAVMAAEKMKILELPYASGEL |
| | | SMLVLLPDEVSGLEQLENAITVEKLMEWTSSSPMEERIMK |
| | | VYLPRMKIEEKYNLTSVLMALGITDLFSSSANLSGISAEE |
| | | SLKMSEAVHQAFAEISEAGSEVVGSSEAGIDATSVSEEFR |
| | | ADHPFLFLIKHNATNSILFFGRCFSP |
| PREDICTED: | SEQ ID NO: 225 | MGSISAASTEFCFDVFKELKVQHVNENIFYSPLSIISALS |
| Ovalbumin like | | MVYLGARENTRAQIEKVVHFDKITGFGESIESQCSTSVSV |
| [Nipponia nippon] | | HTSLKDMFTQITKPSDNYSLSFASRFYAEETYPILPEYLQ |
| | | CVKELYKGGLETINFRTAADQARELINSWVESQTNGMIKN |
| | | ILQPGSVDPQTDMVLVNAIYFKGMWEKAFKDEDTQALPFR |
| | | VTEQESKPVQMMYQIGSFKVAVLASEKVKILELPYASGQL |
| | | SMLVLLPDDVSGLEQLETAITVEKLMEWTSSNNMEERKIK |
| | | VYLPRIKIEEKYNLTSVLMALGITDLFSSSANLSGISSAE |
| | | SLKVSEAIHEAFVEIYEAGSEVAGSTEAGIEVTSVSEEFR |
| | | ADHPFLFLIKHNATNSILFFGRCFSP |
| PREDICTED: | SEQ ID NO: 226 | MVSIGAASTEFCFDVFKELKVQHVNENIFYSPLSIISALS |
| Ovalbumin-like | | MVYLGARENTRAQIDKVVHFDKITGFEETIESQCSTSVSV |
| isoform X2 [Gavia | | HTSLKDMFTQITKPSDNYSLSFASRLYAEETYPILPEYLQ |
| stellata] | | CVKELYKGGLETISFQTAADQARELINSWVESQTDGMIKN |
| | | ILQPGSVDPQTEMVLVNAIYFKGMWEKAFKDEDTQAVPFR |
| | | MTEQESKPVQMMYQIGSFKVAVMASEKMKILELPYASGGM |
| | | SMLVMLPDDVSGLEQLETAITFEKLMEWTSSNMMEERKMK |
| | | VYLPRMKMEEKYNLTSVLMALGMTDLFSSSANLSGISSAE |
| | | SLKMSEAVHEAFVEIYEAGSEAVGSTGAGMEVTSVSEEFR |
| | | ADHPFLFLIKHNPTNSILFFGRCFSP |
| PREDICTED: | SEQ ID NO: 227 | MGSIGAASTEFCFDVFKELKVQHVNENIFYSPLSIISALS |
| Ovalbumin [Pelecanus | | MVYLGARENTRAQIDKVVHFDKITGFGEPIESQCGISVSV |
| crispus] | | HTSLKDMITQITKPSDNYSLSFASRLYAEETYPILPEYLQ |
| | | CVKELYKGGLETISFQTAADQARELINSWVENQTNGMIKN |
| | | ILQPGSVDPQTEMVLVNAVYFKGMWEKAFKDEDTQAVPFR |
| | | MTEQESKPVQMMYQIGSFKVAVMASEKIKILELPYASGEL |
| | | SMLVLLPDDVSGLEQLETAITLDKLTEWTSSNAMEERKMK |
| | | VYLPRMKIEKKYNLTSVLIALGMTDLFSSSANLSGISSAE |
| | | SLKMSEAIHEAFLEIYEAGSEVVGSTEAGMEVTSVSEEFR |
| | | ADHPFLFLIKHNPTNSILFFGRCLSP |
| PREDICTED: | SEQ ID NO: 228 | MGSIGAASTEFCFDVFKELKVQHVNENIFYSPLTIISALS |
| Ovalbumin-like | | MVYLGARENTRAQIDKVVHFDKIPGFGDTTESQCGTSVSV |
| [Charadrius vociferus] | | HTSLKDMFTQITKPSDNYSVSFASRLYAEETYPILPEFLE |
| | | CVKELYKGGLESISFQTAADQARELINSWVESQTNGMIKN |
| | | ILQPGSVDSQTEMVLVNAIYFKGMWEKAFKDEDTQTVPFR |
| | | MTEQETKPVQMMYQIGTFKVAVMPSEKMKILELPYASGEL |
| | | CMLVMLPDDVSGLEELESSITVEKLMEWTSSNMMEERKMK |
| | | VFLPRMKIEEKYNLTSVLMALGMTDLFSSSANLSGISSAE |
| | | PLKMSEAVHEAFIEIYEAGSEVVGSTGAGMEITSVSEEFR |
| | | ADHPFLFLIKHNPTNSILFFGRCVSP |
| PREDICTED: | SEQ ID NO: 229 | MGSIGAVSTEFCFDVFKELKVQHVNENIFYSPLSIISALS |
| Ovalbumin-like | | MVYLGARENTRAQIDKVVHFDKITGSGETIEAQCGTSVSV |
| [Eurypyga helias] | | HTSLKDMFTQITKPSENYSVGFASRLYADETYPIIPEYLQ |
| | | CVKELYKGGLEMISFQTAADQARELINSWVESQTNGMIKN |
| | | ILQPGSVDPQTEMILVNAIYFKGVWEKAFKDEDTQAVPFR |
| | | MTEQESKPVQMMYQFGSFKVAAMAAEKMKILELPYASGAL |
| | | SMLVLLPDDVSGLEQLESAITFEKLMEWTSSNMMEEKKIK |
| | | VYLPRMKMEEKYNFTSVLMALGMTDLFSSSANLSGISSAD |
| | | SLKMSEVVHEAFVEIYEAGSEVVGSTGSGMEAASVSEEFR |
| | | ADHPFLFLIKHNPTNSILFFGRCFSP |
| PREDICTED: | SEQ ID NO: 230 | MVSIGAASTEFCFDVFKELKVQHVNENIFYSPLSIISALS |
| Ovalbumin-like | | MVYLGARENTRAQIDKVVHFDKITGFEETIESQVQKKQCS |
| isoform X1 [Gavia | | TSVSVHTSLKDMFTQITKPSDNYSLSFASRLYAEETYPIL |
| stellata] | | PEYLQCVKELYKGGLETISFQTAADQARELINSWVESQTD |
| | | GMIKNILQPGSVDPQTEMVLVNAIYFKGMWEKAFKDEDTQ |
| | | AVPFRMTEQESKPVQMMYQIGSFKVAVMASEKMKILELPY |
| | | ASGGMSMLVMLPDDVSGLEQLETAITFEKLMEWTSSNMME |
| | | ERKMKVYLPRMKMEEKYNLTSVLMALGMTDLFSSSANLSG |
| | | ISSAESLKMSEAVHEAFVEIYEAGSEAVGSTGAGMEVTSV |
| | | SEEFRADHPFLFLIKHNPTNSILFFGRCFSP |
| PREDICTED: | SEQ ID NO: 231 | MGSIGAASGEFCFDVFKELKVQHVNENIFYSPLSIISALS |
| Ovalbumin -like | | MVYLGARENTRAQIDKVVHFDKIIGFGESIESQCGTSVSV |
| [Egretta garzetta] | | HTSLKDMFAQITKPSDNYSLSFASRLYAEETFPILPEYLQ |
| | | CVKELYKGGLETLSFQTAADQARELINSWVESQTNGMIKD |
| | | ILQPGSVDPQTEMVLVNAIYFKGVWEKAFKDEDTQTVPFR |
| | | MTEQESKPVQMMYQIGSFKVAVVAAEKIKILELPYASGAL |
| | | SMLVLLPDDVSSLEQLETAITFEKLTEWTSSNIMEERKIK |
| | | VYLPRMKIEEKYNLTSVLMDLGITDLFSSSANLSGISSAE |
| | | SLKVSEAIHEAIVDIYEAGSEVVGSSGAGLEGTSVSEEFR |
| | | ADHPFLFLIKHNPTSSILFFGRCFSP |
| PREDICTED: | SEQ ID NO: 232 | MGSIGAASTEFCFDVFKELKVQHVNENIFYSPLSIISALS |
| Ovalbumin-like | | MVYLGARENTRAQIDKVVHFDKITGSGEAIESQCGTSVSV |
| [Balearica regulorum | | HISLKDMFTQITKPSDNYSLSFASRLYAEETYPILPEYLQ |
| gibbericeps] | | CVKELYKEGLATISFQTAADQAREFINSWVESQTNGMIKN |
| | | ILQPGSVDPQTQMVLVNAIYFKGVWEKAFKDEDTQAVPFR |
| | | MTKQESKPVQMMYQIGSFKVAVMASEKMKILELPYASGQL |
| | | SMLVMLPDDVSGLEQIENAITFEKLMEWTNPNMMEERKMK |
| | | VYLPRMKMEEKYNLTSVLMALGMTDLFSSSANLSGISSAE |
| | | SLKMSEAVHEAFVEIYEAGSEVVGSTGAGIEVTSVSEEFR |
| | | ADHPFLFLIKHNPTNSILFFGRCFSP |
| PREDICTED: | SEQ ID NO: 233 | MGSIGEASTEFCIDVFRELKVQHVNENIFYSPLSIISALS |
| Ovalbumin-like | | MVYLGARENTRAQIDQVVHFDKITGFGDTVESQCGSSLSV |
| [Nestor notabilis] | | HSSLKDIFAQITQPKDNYSLNFASRLYAEETYPILPEYLQ |
| | | CVKELYKGGLETISFQTAADQARELINSWVESQTNGMIKN |
| | | ILQPSSVDPQTEMVLVNAIYFKGVWEKAFKDEETQAVPFR |
| | | ITEQENRPVQIMYQFGSFKVAVVASEKIKILELPYASGQL |
| | | SMLVLLPDEVSGLEQLENAITFEKLTEWTSSDIMEEKKIK |
| | | VFLPRMKIEEKYNLTSVLVALGIADLFSSSANLSGISSAE |
| | | SLKMSEAVHEAFVEIYEAGSEVVGSSGAGIEAASDSEEFR |
| | | ADHPFLFLIKHKPTNSILFFGRCFSP |
| PREDICTED: | SEQ ID NO: 234 | MGSIGAASTEFCFDIFNELKVQHVNENIFYSPLSIISALS |
| Ovalbumin-like | | MVYLGARENTKAQIDKVVHFDKITGFGESIESQCSTSASV |
| [Pygoscelis adeliae] | | HTSFKDMFTQITKPSDNYSLSFASRLYAEETYPILPEYSQ |
| | | CVKELYKGGLESISFQTAADQARELINSWVESQTNGMIKN |
| | | ILQPGSVDPQTELVLVNAIYFKGTWEKAFKDKDTQAVPFR |
| | | VTEQESKPVQMMYQIGSYKVAVIASEKMKILELPYASGEL |
| | | SMLVLLPDDVSGLEQLETAITFEKLMEWTSSNMMEERKVK |
| | | VYLPRMKIEEKYNLTSVLMALGMTDLFSPSANLSGISSAE |
| | | SLKMSEAIHEAFVEIYEAGSEVVGSTEAGMEVTSVSEEFR |
| | | ADHPFLFLIKCNLTNSILFFGRCFSP |
| Ovalbumin-like | SEQ ID NO: 235 | MGSISTASTEFCFDVFKELKVQHVNENIFYSPLSIISALS |
| [Athene cunicularia] | | MVYLGARENTRAQIEKVVHFDKITGFGESIESQCGTSVSV |
| | | HTSLKDMLIQISKPSDNYSLSFASKLYAEETYPILPEYLQ |
| | | CVKELYKGGLESINFQTAADQARQLINSWVESQTNGMIKD |
| | | ILQPSSVDPQTEMVLVNAIYFKGIWEKAFKDEDTQEVPFR |
| | | ITEQESKPVQMMYQIGSFKVAVIASEKIKILELPYASGEL |
| | | SMLIVLPDDVSGLEQLETAITFEKLIEWTSPSIMEERKTK |
| | | VYLPRMKIEEKYNLTSVLMALGMTDLFSPSANLSGISSAE |
| | | SLKMSEAIHEAFVEIYEAGSEVVGSAEAGMEATSVSEFRV |
| | | DHPFLFLIKHNPANIILFFGRCVSP |
| PREDICTED: | SEQ ID NO: 236 | MGSIGAASTEFCFDVFKELKVQHVNENIFYSPLTIISALS |
| Ovalbumin-like | | LVYLGARENTRAQIDKVFHFDKISGFGETTESQCGTSVSV |
| [Calidris pugnax] | | HTSLKEMFTQITKPSDNYSVSFASRLYAEDTYPILPEYLQ |
| | | CVKELYKGGLETISFQTAADQAREVINSWVESQTNGMIKN |
| | | ILQPGSVDSQTEMVLVNAIYFKGMWEKAFKDEDTQTMPFR |
| | | ITEQERKPVQMMYQAGSFKVAVMASEKMKILELPYASGEF |
| | | CMLIMLPDDVSGLEQLENSFSFEKLMEWTTSNMMEERKMK |
| | | VYIPRMKMEEKYNLTSVLMALGMTDLFSSSANLSGISSAE |
| | | TLKMSEAVHEAFMEIYEAGSEVVGSTGSGAEVTGVYEEFR |
| | | ADHPFLFLVKHKPTNSILFFGRCVSP |
| PREDICTED: | SEQ ID NO: 237 | MGSIGAASTEFCFDIFNELKVQHVNENIFYSPLSIISALS |
| Ovalbumin | | MVYLGARENTKAQIDKVVHFDKITGFGETIESQCSTSVSV |
| [Aptenodytes forsteri] | | HTSLKDTFTQITKPSDNYSLSFASRLYAEETYPILPEYSQ |
| | | CVKELYKGGLETISFQTAADQARELINSWVESQTNGMIKN |
| | | ILQPGSVDPQTELVLVNAIYFKGTWEKAFKDKDTQAVPFR |
| | | VTEQESKPVQMMYQIGSYKVAVIASEKMKILELPYASREL |
| | | SMLVLLPDDVSGLEQLETAITFEKLMEWTSSNMMEERKVK |
| | | VYLPRMKIEEKYNLTSVLMALGMTDLFSPSANLSGISSAE |
| | | SLKMSEAVHEAFVEIYEAGSEVVGSTGAGMEVTSVSEEFR |
| | | ADHPFLFLIKCNPTNSILFFGRCFSP |
| PREDICTED: | SEQ ID NO: 238 | MGSISAASAEFCLDVFKELKVQHVNENIFYSPLSIISALS |
| Ovalbumin-like | | MVYLGARENTRAQIDKVVHFDKITGSGETIEFQCGTSANI |
| [Pterocles gutturalis] | | HPSLKDMFTQITRLSDNYSLSFASRLYAEERYPILPEYLQ |
| | | CVKELYKGGLETISFQTAADQARELINSWVESQTNGMIKN |
| | | ILQPGSVNPQTEMVLVNAIYFKGLWEKAFKDEDTQTVPFR |
| | | MTEQESKPVQMMYQVGSFKVAVMASDKIKILELPYASGEL |
| | | SMLVLLPDDVTGLEQLETSITFEKLMEWTSSNVMEERTMK |
| | | VYLPHMRMEEKYNLTSVLMALGVTDLFSSSANLSGISSAE |
| | | SLKMSEAVHEAFVEIYESGSQVVGSTGAGTEVTSVSEEFR |
| | | VDHPFLFLIKHNPTNSILFFGRCFSP |
| Ovalbumin-like [Falco | SEQ ID NO: 239 | MGSIGAASVEFCFDVFKELKVQHVNENIFYSPLSIISALS |
| peregrinus] | | MVYLGARENTKAQIDKVVHFDKIAGFGEAIESQCVTSASI |
| | | HSLKDMFTQITKPSDNYSLSFASRLYAEEAYSILPEYLQC |
| | | VKELYKGGLETISFQTAADQARDLINSWVESQTNGMIKNI |
| | | LQPGAVDLETEMVLVNAIYFKGMWEKAFKDEDTQTVPFRM |
| | | TEQESKPVQMMYQVGSFKVAVMASDKIKILELPYASGQLS |
| | | MVVVLPDDVSGLEQLEASITSEKLMEWTSSSIMEEKKIKV |
| | | YFPHMKIEEKYNLTSVLMALGMTDLFSSSANLSGISSAEK |
| | | LKVSEAVHEAFVEISEAGSEVVGSTEAGTEVTSVSEEFKA |
| | | DHPFLFLIKHNPTNSILFFGRCFSP |
| PREDICTED: | SEQ ID NO: 240 | MGSIGAASSEFCFDIFKELKVQHVNENIFYSPLSIISALS |
| Ovalbumin -like | | MVYLGARENTRAQIDKVVPFDKITASGESIESQCSTSVSV |
| isoform X2 | | HTSLKDIFTQITKSSDNHSLSFASRLYAEETYPILPEYLQ |
| [Phalacrocorax carbo] | | CVKELYEGGLETISFQTAADQARELINSWIESQTNGRIKN |
| | | ILQPGSVDPQTEMVLVNAIYFKGMWEKAFKDEDTQAVPFR |
| | | MTEQESKPVQVMHQIGSFKVAVLASEKIKILELPYASGEL |
| | | SMLVLLPDDVSGLEQLETAITFEKLMEWTSPNIMEERKIK |
| | | VFLPRMKIEEKYNLTSVLMALGITDLFSPLANLSGISSAE |
| | | SLKMSEAIHEAFVEISEAGSEVIGSTEAEVEVTNDPEEFR |
| | | ADHPFLFLIKHNPTNSILFFGRCFSP |
| PREDICTED: | SEQ ID NO: 241 | MGSIGAASTEFCFDVFKELKAQYVNENIFYSPMTIITALS |
| Ovalbumin-like | | MVYLGSKENTRAQIAKVAHFDKITGFGESIESQCGASASI |
| [Merops nubicus] | | QFSLKDLFTQITKPSGNHSLSVASRIYAEETYPILPEYLE |
| | | CMKELYKGGLETINFQTAANQARELINSWVERQTSGMIKN |
| | | ILQPSSVDSQTEMVLVNAIYFRGLWEKAFKVEDTQATPFR |
| | | ITEQESKPVQMMHQIGSFKVAVVASEKIKILELPYASGRL |
| | | TMLVVLPDDVSGLKQLETTITFEKLMEWTTSNIMEERKIK |
| | | VYLPRMKIEEKYNLTSVLMALGLTDLFSSSANLSGISSAE |
| | | SLKMSEAVHEAFVEIYEAGSEVVASAEAGMDATSVSEEFR |
| | | ADHPFLFLIKDNTSNSILFFGRCFSP |
| PREDICTED: | SEQ ID NO: 242 | MGSIGAASTEFCFDVFKELKGQHVNENIFFCPLSIVSALS |
| Ovalbumin-like | | MVYLGARENTRAQIVKVAHFDKIAGFAESIESQCGTSVSI |
| [Tauraco | | HTSLKDMFTQITKPSDNYSLNFASRLYAEETYPIIPEYLQ |
| erythrolophus] | | CVKELYKGGLETISFQTAADQAREIINSWVESQTNGMIKN |
| | | ILRPSSVHPQTELVLVNAVYFKGTWEKAFKDEDTQAVPFR |
| | | ITEQESKPVQMMYQI |
| | | GSFKVAAVTSEKMKILEVPYASGELSMLVLLPDDVSGLEQ |
| | | LETAITAEKLIEWTSSTVMEERKLKVYLPRMKIEEKYNLT |
| | | TVLTALGVTDLFSSSANLSGISSAQGLKMSNAVHEAFVEI |
| | | YEAGSEVVGSKGEGTEVSSVSDEFKADHPFLFLIKHNPTN |
| | | SIVFFGRCFSP |
| PREDICTED: | SEQ ID NO: 243 | MGSIGAASTEFCFDVFKELKVHHVNENILYSPLAIISALS |
| Ovalbumin -like | | MVYLGAKENTRDQIDKVVHFDKITGIGESIESQCSTAVSV |
| [Cuculus canorus] | | HTSLKDVFDQITRPSDNYSLAFASRLYAEKTYPILPEYLQ |
| | | CVKELYKGGLETIDFQTAADQARQLINSWVEDETNGMIKN |
| | | ILRPSSVNPQTKIILVNAIYFKGMWEKAFKDEDTQEVPFR |
| | | ITEQETKSVQMMYQIGSFKVAEVVSDKMKILELPYASGKL |
| | | SMLVLLPDDVYGLEQLETVITVEKLKEWTSSIVMEERITK |
| | | VYLPRMKIMEKYNLTSVLTAFGITDLFSPSANLSGISSTE |
| | | SLKVSEAVHEAFVEIHEAGSEVVGSAGAGIEATSVSEEFK |
| | | ADHPFLFLIKHNPTNSILFFGRCFSP |
| Ovalbumin | SEQ ID NO: 244 | MGSIGAASTEFCLDVFKELKVQHVNENIFYSPLSIISALS |
| [Antrostomus | | MVYLGARENTRAQIDKVVHFDKITGFEDSIESQCGTSVSV |
| carolinensis] | | HTSLKDMFTQITKPSDNYSVGFASRLYAAETYQILPEYSQ |
| | | CVKELYKGGLETINFQKAADQATELINSWVESQTNGMIKN |
| | | ILQPSSVDPQTQIFLVNAIYFKGMWQRAFKEEDTQAVPFR |
| | | ISEKESKPVQMMYQIGSFKVAVIPSEKIKILELPYASGLL |
| | | SMLVILPDDVSGLEQLENAITLEKLMQWTSSNMMEERKIK |
| | | VYLPRMRMEEKYNLTSVFMALGITDLFSSSANLSGISSAE |
| | | SLKMSDAVHEASVEIHEAGSEVVGSTGSGTEASSVSEEFR |
| | | ADHPYLFLIKHNPTDSIVFFGRCFSP |
| PREDICTED: | SEQ ID NO: 245 | MGSIGAASTEFCFDVFKELKFQHVDENIFYSPLTIISALS |
| Ovalbumin-like | | MVYLGARENTRAQIDKVVHFDKIAGFEETVESQCGTSVSV |
| [Opisthocomus | | HTSLKDMFAQITKPSDNYSLSFASRLYAEETYPILPEYLQ |
| hoazin] | | CVKELYKGGLETISFQTAADQARDLINSWVESQTNGMIKN |
| | | ILQPSSVGPQTELILVNAIYFKGMWQKAFKDEDTQEVPFR |
| | | MTEQQSKPVQMMYQTGSFKVAVVASEKMKILALPYASGQL |
| | | SLLVMLPDDVSGLKQLESAITSEKLIEWTSPSMMEERKIK |
| | | VYLPRMKIEEKYNLTSVLMALGITDLFSPSANLSGISSAE |
| | | SLKMSQAVHEAFVEIYEAGSEVVGSTGAGMEDSSDSEEFR |
| | | VDHPFLFFIKHNPTNSILFFGRCFSP |
| PREDICTED: | SEQ ID NO: 246 | MGSIGPLSVEFCCDVFKELRIQHPRENIFYSPVTIISALS |
| Ovalbumin-like | | MVYLGARDNTKAQIEKAVHFDKIPGFGESIESQCGTSLSI |
| [Lepidothrix coronata] | | HTSLKDIFTQITKPSDNYTVGIASRLYAEEKYPILPEYLQ |
| | | CIKELYKGGLEPINFQTAAEQARELINSWVESQTNGMIKN |
| | | ILQPSSVNPETDMVLVNAIYFKGLWEKAFKDEDIQTVPFR |
| | | ITEQESKPVQMMFQIGSFRVAEITSEKIRILELPYASGQL |
| | | SLWVLLPDDISGLEQLETAITFENLKEWTSSTKMEERKIK |
| | | VYLPRMKIEEKYNLTSVLTSLGITDLFSSSANLSGISSAE |
| | | SLKVSSAFHEASVEIYEAGSKVVGSTGAEVEDTSVSEEFR |
| | | ADHPFLFLIKHNPSNSIFFFGRCFSP |
| PREDICTED: | SEQ ID NO: 247 | MGSIGTASAEFCFDVFKELKVHHVNENIFYSPLSIISALS |
| Ovalbumin [Struthio | | MVYLGARENTKTQMEKVIHFDKITGLGESMESQCGTGVSI |
| camelus australis] | | HTALKDMLSEITKPSDNYSLSLASRLYAEQTYAILPEYLQ |
| | | CIKELYKESLETVSFQTAADQARELINSWIESQTNGVIKN |
| | | FLQPGSVDSQTELVLVNAIYFKGMWEKAFKDEDTQEVPFR |
| | | ITEQESRPVQMMYQ |
| | | AGSFKVATVAAEKIKILELPYASGELSMLVLLPDDISGLE |
| | | QLETTISFEKLTEWTSSNMMEDRNMKVYLPRMKIEEKYNL |
| | | TSVLIALGMTDLFSPAANLSGISAAESLKMSEAIHAAYVE |
| | | IYEADSEIVSSAGVQVEVTSDSEEFRVDHPFLFLIKHNPT |
| | | NSVLFFGRCISP |
| PREDICTED: | SEQ ID NO: 248 | MGSIGAVSTEFSCDVFKELRIHHVQENIFYSPVTIISALS |
| Ovalbumin-like | | MIYLGARDSTKAQIEKAVHFDKIPGFGESIESQCGTSLSI |
| [Acanthisitta chloris] | | HTSIKDMFTKITKASDNYSIGIASRLYAEEKYPILPEYLQ |
| | | CVKELYKGGLESISFQTAAEQAREIINSWVESQTNGMIKN |
| | | ILQPSSVDPQTDIVLVNAIYFKGLWEKAFRDEDTQTVPFK |
| | | ITEQESKPVQMMYQIGSFKVAEITSEKIKILEVPYASGQL |
| | | SLWVLLPDDISGLEKLETAITFENLKEWTSSTKMEERKIK |
| | | VYLPRMKIEEKYNLTSVLTALGITDLFSSSANLSGISSAE |
| | | SLKVSEAFHEAIVEISEAGSKVVGSVGAGVDDTSVSEEFR |
| | | ADHPFLFLIKHNPTSSIFFFGRCFSP |
| PREDICTED: | SEQ ID NO: 249 | MGSIGAASTEFCFDVFKELKVQHVNENIFYSPLSIISALS |
| Ovalbumin-like [Tyto | | MVYLGARENTRAQIDKVVHFDKIAGFGESTESQCGTSVSA |
| alba] | | HTSLKDMSNQITKLSDNYSLSFASRLYAEETYPILPEYSQ |
| | | CVKELYKGGLESISFQTAAYQARELINAWVESQTNGMIKD |
| | | ILQPGSVDSQTKMVLVNAIYFKGIWEKAFKDEDTQEVPFR |
| | | MTEQETKPVQMMYQIGSFKVAVIAAEKIKILELPYASGQL |
| | | SMLVILPDDVSGLEQLETAITFEKLTEWTSASVMEERKIK |
| | | VYLPRMSIEEKYNLTSVLIALGVTDLFSSSANLSGISSAE |
| | | SLRMSEAIHEAFVETYEAGSTESGTEVTSASEEFRVDHPF |
| | | LFLIKHKPTNSILFFGRCFSP |
| PREDICTED: | SEQ ID NO: 250 | MGSIGAASSEFCFDIFKELKVQHVNENIFYSPLSIISALS |
| Ovalbumin -like | | MVYLGARENTRAQIDKVVPFDKITASGESIESQVQKIQCS |
| isoform X1 | | TSVSVHTSLKDIFTQITKSSDNHSLSFASRLYAEETYPIL |
| [Phalacrocorax carbo] | | PEYLQCVKELYEGGLETISFQTAADQARELINSWIESQTN |
| | | GRIKNILQPGSVDPQTEMVLVNAIYFKGMWEKAFKDEDTQ |
| | | AVPFRMTEQESKPVQVMHQIGSFKVAVLASEKIKILELPY |
| | | ASGELSMLVLLPDDVSGLEQLETAITFEKLMEWTSPNIME |
| | | ERKIKVFLPRMKIEEKYNLTSVLMALGITDLFSPLANLSG |
| | | ISSAESLKMSEAIHEAFVEISEAGSEVIGSTEAEVEVIND |
| | | PEEFRADHPFLFLIKHNPTNSILFFGRCFSP |
| Ovalbumin-like [Pipra | SEQ ID NO: 251 | MGSIGPLSVEFCCDVFKELRIQHARENIFYSPVTIISALS |
| filicauda] | | MVYLGARDNTKAQIEKAVHFDKIPGFGESIESQCGTSLSI |
| | | HTSLKDIFTQITKPSDNYTVGIASRLYAEEKYPILPEYLQ |
| | | CIKELYKGGLEPISFQTAAEQARELINSWVESQTNGIIKN |
| | | ILQPSSVNPETDMVLVNAIYFKGLWEKAFKDEGTQTVPFR |
| | | ITEQESKPVQMMFQIGSFRVAEIASEKIRILELPYASGQL |
| | | SLWVLLPDDISGLEQLETAITFENLKEWTSSTKMEERKIK |
| | | VYLPRMKIEEKYNLTSVLTSLGITDLFSSSANLSGISSAE |
| | | RLKVSSAFHEASMEINEAGSKVVGAGVDDTSVSEEFRVDR |
| | | PFLFLIKHNPSNSIFFFGRCFSP |
| Ovalbumin [Dromaius | SEQ ID NO: 252 | MGSIGAASTEFCFDMFKELKVHHVNENIIYSPLSIISILS |
| novaehollandiae] | | MVFLGARENTKTQMEKVIHFDKITGFGESLESQCGTSVSV |
| | | HASLKDILSEITKPSDNYSLSLASKLYAEETYPVLPEYLQ |
| | | CIKELYKGSLETVSFQTAADQARELINSWVETQTNGVIKN |
| | | FLQPGSVDPQTEMVLVDAIYFKGTWEKAFKDEDTQEVPFR |
| | | ITEQESKPVQMMYQAGSFKVATVAAEKMKILELPYASGEL |
| | | SMFVLLPDDISGLEQLETTISIEKLSEWTSSNMMEDRKMK |
| | | VYLPHMKIEEKYNLTSVLVALGMTDLFSPSANLSGISTAQ |
| | | TLKMSEAIHGAYVEIYEAGSEMATSTGVLVEAASVSEEFR |
| | | VDHPFLFLIKHNPSNSILFFGRCIFP |
| Chain A, Ovalbumin | SEQ ID NO: 253 | MGSIGAASTEFCFDMFKELKVHHVNENIIYSPLSIISILS |
| | | MVFLGARENTKTQMEKVIHFDKITGFGESLESQCGTSVSV |
| | | HASLKDILSEITKPSDNYSLSLASKLYAEETYPVLPEYLQ |
| | | CIKELYKGSLETVSFQTAADQARELINSWVETQTNGVIKN |
| | | FLQPGSVDPQTEMVLVDAIYFKGTWEKAFKDEDTQEVPFR |
| | | ITEQESKPVQMMYQAGSFKVATVAAEKMKILELPYASGEL |
| | | SMFVLLPDDISGLEQLETTISIEKLSEWTSSNMMEDRKMK |
| | | VYLPHMKIEEKYNLTSVLVALGMTDLFSPSANLSGISTAQ |
| | | TLKMSEAIHGAYVEIYEAGSEMATSTGVLVEAASVSEEFR |
| | | VDHPFLFLIKHNPSNSILFFGRCIFPHHHHHH |
| Ovalbumin-like | SEQ ID NO: 254 | MGSIGPLSVEFCCDVFKELRIQHARENIFYSPVTIISALS |
| [Corapipo altera] | | MVYLGARDNTKAQIEKAVHFDKIPGFGESIESQCGTSLSI |
| | | HTSLKDIFTQITKPSDNYTVGIASRLYAEEKYPILPEYLQ |
| | | CIKELYKGGLEPISFQTAAEQARELINSWVESQTNGMIKN |
| | | ILQPSAVNPETDMVLVNAIYFKGLWEKAFKDEGTQTVPFR |
| | | ITEQESKPVQMMFQIGSFRVAEITSEKIRILELPYASGQL |
| | | SLWVLLPDDISGLEQLETAITFENLKEWTSSTKMEERKIK |
| | | VYLPRMKIEEKYNLTSVLTSLGITDLFSSSANLSGISSAE |
| | | RLKVSSAFHEASMEIYEAGSKVVGSTGAGVDDTSVSEEFR |
| | | VDRPFLFLIKHNPSNSIFFFGRCFSP |
| Ovalbumin-like | SEQ ID NO: 255 | MEDQRGNTGFTMGSIGAASTEFCIDVFRELRVQHVNENIF |
| protein [Amazona | | YSPLTIISALSMVYLGARENTRAQIDQVVHFDKIAGFGDT |
| aestiva] | | VESQCGSSPSVHNSLKTVXAQITQPRDNYSLNLASRLYAE |
| | | ESYPILPEYLQCVKELYNGGLETVSFQTAADQARELINSW |
| | | VESQTNGIIKNILQPSSVDPQTEMVLVNAIYFKGLWEKAF |
| | | KDEETQAVPFRITEQENRPVQMMYQFGSFKVAXVASEKIK |
| | | ILELPYASGQLSMLVLLPDEVSGLEQNAITFEKLTEWTSS |
| | | DLMEERKIKVFFPRVKIEEKYNLTAVLVSLGITDLFSSSA |
| | | NLSGISSAENLKMSEAVHEAXVEIYEAGSEVAGSSGAGIE |
| | | VASDSEEFRVDHPFLFLIXHNPTNSILFFGRCFSP |
| PREDICTED: | SEQ ID NO: 256 | MGSIGAASTEFCIDVFRELRVQHVNENIFYSPLSIISALS |
| Ovalbumin-like | | MVYLGARENTRAQIDEVFHFDKIAGFGDTVDPQCGASLSV |
| [Melopsittacus | | HKSLQNVFAQITQPKDNYSLNLASRLYAEESYPILPEYLQ |
| undulatus] | | CVKELYNEGLETVSFQTGADQARELINSWVENQTNGVIKN |
| | | ILQPSSVDPQTEMVLVNAIYFKGLWQKAFKDEETQAVPFR |
| | | ITEQENRPVQMMYQFGSFKVAVVASEKVKILELPYASGQL |
| | | SMWVLLPDEVSGLEQLENAITFEKLTEWTSSDLTEERKIK |
| | | VFLPRVKIEEKYNLTAVLMALGVTDLFSSSANFSGISAAE |
| | | NLKMSEAVHEAFVEIYEAGSEVVGSSGAGIEAPSDSEEFR |
| | | ADHPFLFLIKHNPTNSILFFGRCFSP |
| Ovalbumin-like | SEQ ID NO: 257 | MGSIGPLSVEFCCDVFKELRIQHARDNIFYSPVTIISALS |
| [Neopelma | | MVYLGARDNTKAQIEKAVHFDKIPGFGESIESQCGTSLSV |
| chrysocephalum] | | HTSLKDIFTQITKPRENYTVGIASRLYAEEKYPILPEYLQ |
| | | CIKELYKGGLEPISFQTAAEQARELINSWVESQTNGMIKN |
| | | ILQPSSVNPETDMVLVNAIYFKGLWKKAFKDEGTQTVPFR |
| | | ITEQESKPVQMMFQIGSFRVAEITSEKIRILELPYASGQL |
| | | SLWVLLPDDISGLEQLESAITFENLKEWTSSTKMEERKIK |
| | | VYLPRMKIEEKYNLTSVLTSLGITDLFSSSANLSGISSAE |
| | | KLKVSSAFHEASMEIYEAGNKVVGSTGAGVDDTSVSEEFR |
| | | VDRPFLFLIKHNPSNSIFFFGRCFSP |
| PREDICTED: | SEQ ID NO: 258 | MGSIGAASAEFCVDVFKELKDQHVNNIVFSPLMIISALSM |
| Ovalbumin-like | | VNIGAREDTRAQIDKVVHFDKITGYGESIESQCGTSIGIY |
| [Buceros rhinoceros | | FSLKDAFTQITKPSDNYSLSFASKLYAEETYPILPEYLKC |
| silvestris] | | VKELYKGGLETISFQTAADQARELINSWVESQTNGMIKNI |
| | | LQPSSVDPQTEMVLVNAIYFKGLWEKAFKDEDTQAVPFRI |
| | | TEQESKPVQMMYQIGSFKVAVIASEKIKILELPYASGQLS |
| | | LLVLLPDDVSGLEQLESAITSEKLLEWTNPNIMEERKTKV |
| | | YLPRMKIEEKYNLTSVLVALGITDLFSSSANLSGISSAEG |
| | | LKLSDAVHEAFVEIYEAGREVVGSSEAGVEDSSVSEEFKA |
| | | DRPFIFLIKHNPTNGILYFGRYISP |
| PREDICTED: | SEQ ID NO: 259 | MGSIGAANTDFCFDVFKELKVHHANENIFYSPLSIVSALA |
| Ovalbumin-like | | MVYLGARENTRAQIDKALHFDKILGFGETVESQCDTSVSV |
| [Cariama cristata] | | HTSLKDMLIQITKPSDNYSFSFASKIYTEETYPILPEYLQ |
| | | CVKELYKGGVETISFQTAADQAREVINSWVESHTNGMIKN |
| | | ILQPGSVDPQTKMVLVNAVYFKGIWEKAFKEEDTQEMPFR |
| | | INEQESKPVQMMYQIGSFKLTVAASENLKILEFPYASGQL |
| | | SMMVILPDEVSGLKQLETSITSEKLIKWTSSNTMEERKIR |
| | | VYLPRMKIEEKYNLKSVLMALGITDLFSSSANLSGISSAE |
| | | SLKMSEAVHEAFVEIYEAGSEVTSSTGTEMEAENVSEEFK |
| | | ADHPFLFLIKHNPTDSIVFFGRCMSP |
| Ovalbumin [Manacus | SEQ ID NO: 260 | MGSIGPLSVEFCCDVFKELRIQHARENIFYSPVTIISALS |
| vitellinus] | | MVYLGARDNTKAQIEKAVHFDKIPGFGESIESQCGTSLSI |
| | | HTSLKDIFTQITKPSDNYTVGIASRLYAEEKYPILPEYLQ |
| | | CIKELYKGGLEPISFQTAAEQARELINSWVESQTNGMIKN |
| | | ILQPSSVNPETDMVLVNAIYFKGLWEKAFKDESTQTVPFR |
| | | ITEQESKPVQMMFQIGSFRVAEIASEKIRILELPYASGQL |
| | | SLWVLLPDDISGLEQLETAITFENLKEWTSSTKMEERKIK |
| | | VYLPRMKIEEKYNLTSVLTSLGITDLFSSSANLSGISSAE |
| | | RLKVSSAFHEASMEIYEAGSRVVEAGVDDTSVSEEFRVDR |
| | | PFLFLIKHNPSNSIFFFGRCFSP |
| Ovalbumin-like | SEQ ID NO: 261 | MGSIGPVSTEFCCDIFKELRIQHARENIIYSPVTIISALS |
| [Empidonax traillii] | | MVYLGARDNTKAQIEKAVHFDKIPGFGESIESQCGTSLSI |
| | | HTSLKDILTQITKPSDNYTVGIASRLYAEEKYPILSEYLQ |
| | | CIKELYKGGLEPISFQTAAEQARELINSWVESQTNGMIKN |
| | | ILQPSSVNPETDMVLVNAIYFKGLWEKAFKDEGTQTVPFR |
| | | ITEQESKPVQMMFQIGSFKVAEITSEKIRILELPYASGKL |
| | | SLWVLLPDDISGLEQLETAITFENLKEWTSSTRMEERKIK |
| | | VYLPRMKIEEKYNLTSVLTSLGITDLFSSSANLSGISSAE |
| | | RLKVSSAFHEVFVEIYEAGSKVEGSTGAGVDDTSVSEEFR |
| | | ADHPFLFLVKHNPSNSIIFFGRCYLP |
| PREDICTED: | SEQ ID NO: 262 | MGSTGAASMEFCFALFRELKVQHVNENIFFSPVTIISALS |
| Ovalbumin-like | | MVYLGARENTRAQLDKVAPFDKITGFGETIGSQCSTSASS |
| [Leptosomus discolor] | | HTSLKDVFTQITKASDNYSLSFASRLYAEETYPILPEYLQ |
| | | CVKELYKGGLESISFQTAADQARELINSWVESQTNGMIKD |
| | | ILRPSSVDPQTKIILITAIYFKGMWEKAFKEEDTQAVPFR |
| | | MTEQESKPVQMMYQIGSFKVAVIPSEKLKILELPYASGQL |
| | | SMLVILPDDVSGLEQLETAITTEKLKEWTSPSMMKERKMK |
| | | VYFPRMRIEEKYNLTSVLMALGITDLFSPSANLSGISSAE |
| | | SLKVSEAVHEASVDIDEAGSEVIGSTGVGTEVTSVSEEIR |
| | | ADHPFLFLIKHKPTNSILFFGRCFSP |
| Hypothetical protein | SEQ ID NO: 263 | MEHAQLTQLVNSNMTSNTCHEADEFENIDFRMDSISVTNT |
| H355_008077 | | KFCFDVFNEMKVHHVNENILYSPLSILTALAMVYLGARGN |
| [Colinus virginianus] | | TESQMKKALHFDSITGAGSTTDSQCGSSEYIHNLFKEFLT |
| | | EITRTNATYSLEIADKLYVDKTFTVLPEYINCARKFYTGG |
| | | VEEVNFKTAAEEARQLINSWVEKETNGQIKDLLVPSSVDF |
| | | GTMMVFINTIYFKGIWKTAFNTEDTREMPFSMTKQESKPV |
| | | QMMCLNDTFNMATLPAEKMRILELPYASGELSMLVLLPDE |
| | | VSGLEQIEKAINFEKLREWTSTNAMEKKSMKVYLPRMKIE |
| | | EKYNLTSTLMALGMTDLFSRSANLTGISSVENLMISDAVH |
| | | GAFMEVNEEGTEAAGSTGAIGNIKHSVEFEEFRADHPFLF |
| | | LIRYNPTNVILFFDNSEFTMGSIGAVSTEFCFDVFKELRV |
| | | HHANENIFYSPFTVISALAMVYLGAKDSTRTQINKVVRFD |
| | | KLPGFGDSIEAQCGTSANVHSSLRDILNQITKPNDIYSFS |
| | | LASRLYADETYTILPEYLQCVKELYRGGLESINFQTAADQ |
| | | ARELINSWVESQTSGIIRNVLQPSSVDSQTAMVLVNAIYF |
| | | KGLWEKGFKDEDTQAMPFRVTEQENKSVQMMYQIGTFKVA |
| | | SVASEKMKILELPFASGTMSMWVLLPDEVSGLEQLETTIS |
| | | IEKLTEWTSSSVMEERKIKVFLPRMKMEEKYNLTSVLMAM |
| | | GMTDLFSSSANLSGISSTLQKKGFRSQELGDKYAKPMLES |
| | | PALTPQVTAWDNSWIVAHPAAIEPDLCYQIMEQKWKPFDW |
| | | PDFRLPMRVSCRFRTMEALNKANTSFALDFFKHECQEDDD |
| | | ENILFSPFSISSALATVYLGAKGNTADQMAKTEIGKSGNI |
| | | HAGFKALDLEINQPTKNYLLNSVNQLYGEKSLPFSKEYLQ |
| | | LAKKYYSAEPQSVDFLGKANEIRREINSRVEHQTEGKIKN |
| | | LLPPGSIDSLTRLVLVNALYFKGNWATKFEAEDTRHRPFR |
| | | INMHTTKQVPMMYLRDKFNWTYVESVQTDVLELPYVNNDL |
| | | SMFILLPRDITGLQKLINELTFEKLSAWTSPELMEKMKME |
| | | VYLPRFTVEKKYDMKSTLSKMGIEDAFTKVDSCGVTNVDE |
| | | ITTHIVSSKCLELKHIQINKKLKCNKAVAMEQVSASIGNF |
| | | TIDLFNKLNETSRDKNIFFSPWSVSSALALTSLAAKGNTA |
| | | REMAEDPENEQAENIHSGFKELMTALNKPRNTYSLKSANR |
| | | IYVEKNYPLLPTYIQLSKKYYKAEPYKVNFKTAPEQSRKE |
| | | INNWVEKQTERKIKNFLSSDDVKNSTKSILVNAIYFKAEW |
| | | EEKFQAGNTDMQPFRMSKNKSKLVKMMYMRHTFPVLIMEK |
| | | LNFKMIELPYVKRELSMFILLPDDIKDSTTGLEQLERELT |
| | | YEKLSEWADSKKMSVTLVDLHLPKFSMEDRYDLKDALKSM |
| | | GMASAFNSNADFSGMTGFQAVPMESLSASTNSFTLDLYKK |
| | | LDETSKGQNIFFASWSIATALAMVHLGAKGDTATQVAKGP |
| | | EYEETENIHSGFKELLSAINKPRNTYLMKSANRLFGDKTY |
| | | PLLPKFLELVARYYQAKPQAVNFKTDAEQARAQINSWVEN |
| | | ETESKIQNLLPAGSIDSHTVLVLVNAIYFKGNWEKRFLEK |
| | | DTSKMPFRLSKTETKPVQMMFLKDTFLIHHERTMKFKIIE |
| | | LPYVGNELSAFVLLPDDISDNTTGLELVERELTYEKLAEW |
| | | SNSASMMKAKVELYLPKLKMEENYDLKSVLSDMGIRSAFD |
| | | PAQADFTRMSEKKDLFISKVIHKAFVEVNEEDRIVQLASG |
| | | RLTGRCRTLANKELSEKNRTKNLFFSPFSISSALSMILLG |
| | | SKGNTEAQIAKVLSLSKAEDAHNGYQSLLSEINNPDTKYI |
| | | LRTANRLYGEKTFEFLSSFIDSSQKFYHAGLEQTDFKNAS |
| | | EDSRKQINGWVEEKTEGKIQKLLSEGIINSMTKLVLVNAI |
| | | YFKGNWQEKFDKETTKEMPFKINKNETKPVQMMFRKGKYN |
| | | MTYIGDL |
| | | ETTVLEIPYVDNELSMIILLPDSIQDESTGLEKLERELTY |
| | | EKLMDWINPNMMDSTEVRVSLPRFKLEENYELKPTLSTMG |
| | | MPDAFDLRTADFSGISSGNELVLSEVVHKSFVEVNEEGTE |
| | | AAAATAGIMLLRCAMIVANFTADHPFLFFIRHNKTNSILF |
| | | CGRFCSP |
| PREDICTED: | SEQ ID NO: 264 | MGSIGTASTEFCFDMFKEMKVQHANQNIIFSPLTIISALS |
| Ovalbumin isoform | | MVYLGARDNTKAQMEKVIHFDKITGFGESVESQCGTSVSI |
| X2 [Apteryx australis | | HTSLKDMLSEITKPSDNYSLSLASRLYAEETYPILPEYLQ |
| mantelli] | | CMKELYKGGLETVSFQTAADQARELINSWVESQTNGVIKN |
| | | FLQPGSVDPQTEMVLVNAIYFKGMWEKAFKDEDTQEVPFR |
| | | ITEQESKPVQMMYQVGSFKVATVAAEKMKILEIPYTHREL |
| | | SMFVLLPDDISGLEQLETTISFEKLTEWTSSNMMEERKVK |
| | | VYLPHMKIEEKYNLTSVLMALGMTDLFSPSANLSGISTAQ |
| | | TLMMSEAIHGAYVEIYEAGREMASSTGVQVEVTSVLEEVR |
| | | ADKPFLFFIRHNPTNSMVVFGRYMSP |
| Hypothetical protein | SEQ ID NO: 265 | MTSNTCHEADEFEN |
| ASZ78_006007 | | IDFRMDSISVTNTKFCFDVFNEMKVHHVNENILYSPLSIL |
| [Callipepla squamata] | | TALAMVYLGARGNTESQMKKALHFDSITGGGSTTDSQCGS |
| | | SEYIHNLFKEFLTEITRTNATYSLEIADKLYVDKTFTVLP |
| | | EYINCARKFYTGGVEEVNFKTAAEEARQLMNSWVEKETNG |
| | | QIKDLLVPSSVDFGTMMVFINTIYFKGIWKTAFNTEDTRE |
| | | MPFSMTKQESKPVQMMCLNDTFNMVTLPAEKMRILELPYA |
| | | SGELSMLVLLPDEVSGLERIEKAINFEKLREWTSTNAMEK |
| | | KSMKVYLPRMKIEEKYNLTSTLMALGMTDLFSRSANLTGI |
| | | SSVDNLMISDAVHGAFMEVNEEGTEAAGSTGAIGNIKHSV |
| | | EFEEFRADHPFLFLIRYNPTNVILFFDNSEFTMGSIGAVS |
| | | TEFCFDVFKELRVHHANENIFYSPFTIISALAMVYLGAKD |
| | | STRTQINKVVRFDKLPGFGDSIEAQCGTSANVHSSLRDIL |
| | | NQITKPNDIYSFSLASRLYADETYTILPEYLQCVKELYRG |
| | | GLESINFQTAADQARELINSWVESQTSGIIRNVLQPSSVD |
| | | SQTAMVLVNAIYFKGLWEKGFKDEDTQAIPFRVTEQENKS |
| | | VQMMYQIGTFKVASVASEKMKILELPFASGTMSMWVLLPD |
| | | EVSGLEQLETTISIEKLTEWTSSSVMEERKIKVFLPRMKM |
| | | EEKYNLTSVLMAMGMTDLFSSSANLSGISSTLQKKGFRSQ |
| | | ELGDKYAKPMLESPALTPQATAWDNSWIVAHPPAIEPDLY |
| | | YQIMEQKWKPFDWPDFRLPMRVSCRFRTMEALNKANTSFA |
| | | LDFFKHECQEDDSENILFSPFSISSALATVYLGAKGNTAD |
| | | QMAKVLHFNEAEGARNVTTTIRMQVYSRTDQQRLNRRACF |
| | | QKTEIGKSGNIHAGFKGLNLEINQPTKNYLLNSVNQLYGE |
| | | KSLPFSKEYLQLAKKYYSAEPQSVDFVGTANEIRREINSR |
| | | VEHQTEGKIKNLLPPGSIDSLTRLVLVNALYFKGNWATKF |
| | | EAEDTRHRPFRINTHTTKQVPMMYLSDKFNWTYVESVQTD |
| | | VLELPYVNNDLSMFILLPRDITGLQKLINELTFEKLSAWT |
| | | SPELMEKMKMEVYLPRFTVEKKYDMKSTLSKMGIEDAFTK |
| | | VDNCGVTNVDEITIHVVPSKCLELKHIQINKELKCNKAVA |
| | | MEQVSASIGNFTIDLFNKLNETSRDKNIFFSPWSVSSALA |
| | | LTSLAAKGNTAREMAEDPENEQAENIHSGFNELLTALNKP |
| | | RNTYSLKSANRIYVEKNYPLLPTYIQLSKKYYKAEPHKVN |
| | | FKTAPEQSRKEINNWVEKQTERKIKNFLSSDDVKNSTKLI |
| | | LVNAIYFKAEWEEKFQAGNTDMQPFRMSKNKSKLVKMMYM |
| | | RHTFPVLIMEKLNFKMIELPYVKRELSMFILLPDDIKDST |
| | | TGLEQLERELTYEKLSEWADSKKMSVTLVDLHLPKFSMED |
| | | RYDLKDALRSMGMASAFNSNADFSG |
| | | MTGERDLVISKVCHQSFVAVDEKGTEAAAATAVIAEAVPM |
| | | ESLSASTNSFTLDLYKKLDETSKGQNIFFASWSIATALTM |
| | | VHLGAKGDTATQVAKGPEYEETENIHSGFKELLSALNKPR |
| | | NTYSMKSANRLFGDKTYPLLPTKTKPVQMMFLKDTFLIHH |
| | | ERTMKFKIIELPYMGNELSAFVLLPDDISDNTTGLELVER |
| | | ELTYEKLAEWSNSASMMKVKVELYLPKLKMEENYDLKSAL |
| | | SDMGIRSAFDPAQADFTRMSEKKDLFISKVIHKAFVEVNE |
| | | EDRIVQLASGRLTGNTEAQIAKVLSLSKAEDAHNGYQSLL |
| | | SEINNPDTKYILRTANRLYGEKTFEFLSSFIDSSQKFYHA |
| | | GLEQTDFKNASEDSRKQINGWVEEKTEGKIQKLLSEGIIN |
| | | SMTKLVLVNAIYFKGNWQEKFDKETTKEMPFKINKNETKP |
| | | VQMMFRKGKYNMTYIGDLETTVLEIPYVDNELSMIILLPD |
| | | SIQDESTGLEKLERELTYEKLMDWINPNMMDSTEVRVSLP |
| | | RFKLEENYELKPTLSTMGMPDAFDLRTADFSGISSGNELV |
| | | LSEVVHKSFVEVNEEGTEAAAATAGIMLLRCAMIVANFTA |
| | | DHPFLFFIRHNKTNSILFCGRFCSP |
| PREDICTED: | SEQ ID NO: 266 | MASIGAASTEFCFDVFKELKTQHVKENIFYSPMAIISALS |
| Ovalbumin-like | | MVYIGARENTRAEIDKVVHFDKITGFGNAVESQCGPSVSV |
| [Mesitornis unicolor] | | HSSLKDLITQISKRSDNYSLSYASRIYAEETYPILPEYLQ |
| | | CVKEVYKGGLESISFQTAADQARENINAWVESQTNGMIKN |
| | | ILQPSSVNPQTEMVLVNAIYLKGMWEKAFKDEDTQTMPFR |
| | | VTQQESKPVQMMYQIGSFKVAVIASEKMKILELPYTSGQL |
| | | SMLVLLPDDVSGLEQVESAITAEKLMEWTSPSIMEERTMK |
| | | VYLPRMKMVEKYNLTSVLMALGMTDLFTSVANLSGISSAQ |
| | | GLKMSQAIHEAFVEIYEAGSEAVGSTGVGMEITSVSEEFK |
| | | ADLSFLFLIRHNPTNSIIFFGRCISP |
| Ovalbumin, partial | SEQ ID NO: 267 | MGSIGAASTEFCFDVFRELRVQHVNENIFYSPFSIISALA |
| [Anas platyrhynchos] | | MVYLGARDNTRTQIDKISQFQALSDEHLVLCIQQLGEFFV |
| | | CTNRERREVTRYSEQTEDKTQDQNTGQIHKIVDTCMLRQD |
| | | ILTQITKPSDNFSLSFASRLYAEETYAILPEYLQCVKELY |
| | | KGGLESISFQTAADQARELINSWVESQTNGIIKNILQPSS |
| | | VDSQTTMVLVNAIYFKGMWEKAFKDEDTQAMPFRMTEQES |
| | | KPVQMMYQVGSFKVAMVTSEKMKILELPFASGMMSMFVLL |
| | | PDEVSGLEQLESTISFEKLTEWTSSTMMEERRMKVYLPRM |
| | | KMEEKYNLTSVFMALGMTDLFSSSANMSGISSTVSLKMSE |
| | | AVHAACVEIFEAGRDVVGSAEAGMDVTSVSEEFRADHPFL |
| | | FFIKHNPTNSILFFGRWMSP |
| PREDICTED: | SEQ ID NO: 268 | MGSIGAASAEFCLDIFKELKVQHVNENIIFSPMTIISALS |
| Ovalbumin-like | | LVYLGAKEDTRAQIEKVVPFDKIPGFGEIVESQCPKSASV |
| [Chaetura pelagica] | | HSSIQDIFNQIIKRSDNYSLSLASRLYAEESYPIRPEYLQ |
| | | CVKELDKEGLETISFQTAADQARQLINSWVESQTNGMIKN |
| | | ILQPSSVNSQTEMVLVNAIYFRGLWQKAFKDEDTQAVPFR |
| | | ITEQESKPVQMMQQIGSFKVAEIASEKMKILELPYASGQL |
| | | SMLVLLPDDVSGLEKLESSITVEKLIEWTSSNLTEERNVK |
| | | VYLPRLKIEEKYNLTSVLAALGITDLFSSSANLSGISTAE |
| | | SLKLSRAVHESFVEIQEAGHEVEGPKEAGIEVTSALDEFR |
| | | VDRPFLFVTKHNPTNSILFLGRCLSP |
| PREDICTED: | SEQ ID NO: 269 | MGSISAASGEFCLDIFKELKVQHVNENIFYSPMVIVSALS |
| Ovalbumin-like | | LVYLGARENTRAQIDKVIPFDKITGSSEAVESQCGTPVGA |
| [Apaloderma vittatum] | | HISLKDVFAQIAKRSDNYSLSFVNRLYAEETYPILPEYLQ |
| | | CVKELYKGGLETISFQTAADQAREIINSWVESQTDGKIKN |
| | | ILQPSSVDPQTKMVLVSAIYFKGLWEKSFKDEDTQAVPFR |
| | | VTEQESKPVQMMYQIGSFKVAAIAAEKIKILELPYASEQL |
| | | SMLVLLPDDVSGLEQLEKKISYEKLTEWTSSSVMEEKKIK |
| | | VYLPRMKIEEKYNLTSILMSLGITDLFSSSANLSGISSTK |
| | | SLKMSEAVHEASVEIYEAGSEASGITGDGMEATSVFGEFK |
| | | VDHPFLFMIKHKPTNSILFFGRCISP |
| Ovalbumin-like | SEQ ID NO: 270 | MGSIGPVSTEVCCDIFRELRSQSVQENVCYSPLLIISTLS |
| [Corvus cornix cornix] | | MVYIGAKDNTKAQIEKAIHFDKIPGFGESTESQCGTSVSI |
| | | HTSLKDIFTQITKPSDNYSISIARRLYAEEKYPILPEYIQ |
| | | CVKELYKGGLESISFQTAAEKSRELINSWVESQTNGTIKN |
| | | ILQPSSVSSQTDMVLVSAIYFKGLWEKAFKEEDTQTIPFR |
| | | ITEQESKPVQMMSQIGTFKVAEIPSEKCRILELPYASGRL |
| | | SLWVLLPDDISGLEQLETAITFENLKEWTSSSKMEERKIR |
| | | VYLPRMKIEEKYNLTSVLKSLGITDLFSSSANLSGISSAE |
| | | SLKVSAAFHEASVEIYEAGSKGVGSSEAGVDGTSVSEEIR |
| | | ADHPFLFLIKHNPSDSILFFGRCFSP |
| PREDICTED: | SEQ ID NO: 271 | MGSIGAASTEFCFDVFKELKVQHVNENIIISPLSIISALS |
| Ovalbumin-like | | MVYLGAREDTRAQIDKVVHFDKITGFGEAIESQCPTSESV |
| [Calypte anna] | | HASLKETFSQLTKPSDNYSLAFASRLYAEETYPILPEYLQ |
| | | CVKELYKGGLETINFQTAAEQARQVINSWVESQTDGMIKS |
| | | LLQPSSVDPQTEMILVNAIYFRGLWERAFKDEDTQELPFR |
| | | ITEQESKPVQMMSQIGSFKVAVVASEKVKILELPYASGQL |
| | | SMLVLLPDDVSGLEQLESSITVEKLIEWISSNTKEERNIK |
| | | VYLPRMKIEEKYNLTSVLVALGITDLFSSSANLSGISSAE |
| | | SLKISEAVHEAFVEIQEAGSEVVGSPGPEVEVTSVSEEWK |
| | | ADRPFLFLIKHNPTNSILFFGRYISP |
| PREDICTED: | SEQ ID NO: 272 | MGSIGPVSTEVCCDIFRELRSQSVQENVCYSPLLIISTLS |
| Ovalbumin [Corvus | | MVYIGAKDNTKAQIEKAIHFDKIPGFGESTESQCGTSVSI |
| brachyrhynchos] | | HTSLKDIFTQITKPSDNYSISIARRLYAEEKYPILQEYIQ |
| | | CVKELYKGGLESISFQTAAEKSRELINSWVESQTNGTIKN |
| | | ILQPSSVSSQTDMVLVSAIYFKGLWEKAFKEEDTQTIPFR |
| | | ITEQESKPVQMMSQIGTFKVAEIPSEKCRILELPYASGRL |
| | | SLWVLLPDDISGLEQLETSITFENLKEWTSSSKMEERKIR |
| | | VYLPRMKIEEKYNLTSVLKSLGITDLFSSSANLSGISSAE |
| | | SLKVSAVFHEASVEIYEAGSKGVGSSEAGVDGTSVSEEIR |
| | | ADHPFLFLIKHNPSDSILFFGRCFSP |
| Hypothetical protein | SEQ ID NO: 273 | MLNLMHPKQFCCTMGSIGPVSTEVCCDIFRELRSQSVQEN |
| DUI87_08270 | | VCYSPLLIISTLSMVYIGAKDNTKAQIEKAIHFDKIPGFG |
| [Hirundo rustica | | ESTESQCGTSVSIHTSLKDIFTQITKPSDNYSISIASRLY |
| rustica] | | AEEKYPILPEYIQCVKELYKGGLESISFQTAAEKSRELIN |
| | | SWVESQTNGTIKNILQPSSVSSQTDMVLVSAIYFKGLWEK |
| | | AFKEEDTQTVPFRITEQESKPVQMMSQIGTFKVAEIPSEK |
| | | CRILELPYASGRLSLWVLLPDDISGLEQLETAITSENLKE |
| | | WTSSSKMEERKIKVYLPRMKIEEKYNLTSVLKSLGITDLF |
| | | SSSANLSGISSAESLKVSGAFHEAFVEIYEAGSKAVGSSG |
| | | AGVEDTSVSEEIRADHPFLFFIKHNPSDSILFFGRCFSP |
| Ostrich OVA | SEQ ID NO: 274 | EAEAGSIGTASAEFCFDVFKELKVHHVNENIFYSPLSIIS |
| sequence as secreted | | ALSMVYLGARENTKTQMEKVIHFDKITGLGESMESQCGTG |
| from pichia | | VSIHTALKDMLSEITKPSDNYSLSLASRLYAEQTYAILPE |
| | | YLQCIKELYKESLETVSFQTAADQARELINSWIESQTNGV |
| | | IKNFLQPGSVDSQTELVLVNAIYFKGMWEKAFKDEDTQEV |
| | | PFRITEQESRPVQMMYQAGSFKVATVAAEKIKILELPYAS |
| | | GELSMLVLLPDDISGLEQLETTISFEKLTEWTSSNMMEDR |
| | | NMKVYLPRMKIEEKYNLTSVLIALGMTDLFSPAANLSGIS |
| | | AAESLKMSEAIHAAYVEIYEADSEIVSSAGVQVEVTSDSE |
| | | EFRVDHPFLFLIKHNPTNSVLFFGRCISP |
| Ostrich construct | SEQ ID NO: 275 | MRFPSIFTAVLFAASSALAAPVNTTTEDETAQIPAEAVIG |
| (secretion signal | | YSDLEGDFDVAVLPFSNSTNNGLLFINTTIASIAAKEEGV |
| mature protein) | | SLEKREAEAGSIGTASAEFCFDVFKELKVHHVNENIFYSP |
| | | LSIISALSMVYLGARENTKTQMEKVIHFDKITGLGESMES |
| | | QCGTGVSIHTALKDMLSEITKPSDNYSLSLASRLYAEQTY |
| | | AILPEYLQCIKELYKESLETVSFQTAADQARELINSWIES |
| | | QTNGVIKNFLQPGSVDSQTELVLVNAIYFKGMWEKAFKDE |
| | | DTQEVPFRITEQESRPVQMMYQAGSFKVATVAAEKIKILE |
| | | LPYASGELSMLVLLPDDISGLEQLETTISFEKLTEWTSSN |
| | | MMEDRNMKVYLPRMKIEEKYNLTSVLIALGMTDLFSPAAN |
| | | LSGISAAESLKMSEAIHAAYVEIYEADSEIVSSAGVQVEV |
| | | TSDSEEFRVDHPFLFLIKHNPTNSVLFFGRCISP |
| Duck OVA sequence | SEQ ID NO: 276 | EAEAGSIGAASTEFCFDVFRELRVQHVNENIFYSPFSIIS |
| as secreted from pichia | | ALAMVYLGARDNTRTQIDKVVHFDKLPGFGESMEAQCGTS |
| | | VSVHSSLRDILTQITKPSDNFSLSFASRLYAEETYAILPE |
| | | YLQCVKELYKGGLESISFQTAADQARELINSWVESQINGI |
| | | IKNILQPSSVDSQTTMVLVNAIYFKGMWEKAFKDEDTQAM |
| | | PFRMTEQESKPVQMMYQVGSFKVAMVTSEKMKILELPFAS |
| | | GMMSMFVLLPDEVSGLEQLESTISFEKLTEWTSSTMMEER |
| | | RMKVYLPRMKMEEKYNLTSVFMALGMTDLFSSSANMSGIS |
| | | STVSLKMSEAVHAACVEIFEAGRDVVGSAEAGMDVTSVSE |
| | | EFRADHPFLFFIKHNPTNSILFFGRWMSP |
| Duck construct | SEQ ID NO: 277 | MRFPSIFTAVLFAASSALAAPVNTTTEDETAQIPAEAVIG |
| (secretion signal | | YSDLEGDFDVAVLPFSNSTNNGLLFINTTIASIAAKEEGV |
| mature protein) | | SLEKREAEAGSIGAASTEFCFDVFRELRVQHVNENIFYSP |
| | | FSIISALAMVYLGARDNTRTQIDKVVHFDKLPGFGESMEA |
| | | QCGTSVSVHSSLRDILTQITKPSDNFSLSFASRLYAEETY |
| | | AILPEYLQCVKELYKGGLESISFQTAADQARELINSWVES |
| | | QTNGIIKNILQPSSVDSQTTMVLVNAIYFKGMWEKAFKDE |
| | | DTQAMPFRMTEQESKPVQMMYQVGSFKVAMVTSEKMKILE |
| | | LPFASGMMSMFVLLPDEVSGLEQLESTISFEKLTEWTSST |
| | | MMEERRMKVYLPRMKMEEKYNLTSVFMALGMTDLFSSSAN |
| | | MSGISSTVSLKMSEAVHAACVEIFEAGRDVVGSAEAGMDV |
| | | TSVSEEFRADHPFLFFIKHNPTNSILFFGRWMSP |
| Ovoglobulin G2 | SEQ ID NO: 278 | TRAPDCGGILTPLGLSYLAEVSKPHAEVVLRQDLMAQRAS |
| | | DLFLGSMEPSRNRITSVKVADLWLSVIPEAGLRLGIEVEL |
| | | RIAPLHAVPMPVRISIRADLHVDMGPDGNLQLLTSACRPT |
| | | VQAQSTREAESKSSRSILDKVVDVDKLCLDVSKLLLFPNE |
| | | QLMSLTALFPVTPNCQLQYLPLAAPVFSKQGIALSLQTTF |
| | | QVAGAVVPVPVSPVPFSMPELASTSTSHLILALSEHFYTS |
| | | LYFTLERAGAFNMTIPSMLTTATLAQKITQVGSLYHEDLP |
| | | ITLSAALRSSPRVVLEEGRAALKLFLTVHIGAGSPDFQSF |
| | | LSVSADVTAGLQLSVSDTRMMISTAVIEDAELSLAASNVG |
| | | LVRAALLEELFLAPVCQQVPAWMDDVLREGVHLPHLSHFT |
| | | YTDVNVVVHKDYVLVPCKLKLRSTMA* |
| Ovoglobulin G3 | SEQ ID NO: 279 | MDSISVTNAKFCFDVFNEMKVHHVNENILYCPLSILTALA |
| | | MVYLGARGNTESQMKKVLHFDSITGAGSTTDSQCGSSEYV |
| | | HNLFKELLSEITRPNATYSLEIADKLYVDKTFSVLPEYLS |
| | | CARKFYTGGVEEVNFKTAAEEARQLINSWVEKETNGQIKD |
| | | LLVSSSIDFGTTMVFINTIYFKGIWKIAFNTEDTREMPFS |
| | | MTKEESKPVQMMCMNNSFNVATLPAEKMKILELPYASGDL |
| | | SMLVLLPDEVSGLERIEKTINFDKLREWTSTNAMAKKSMK |
| | | VYLPRMKIEEKYNLTSILMALGMTDLFSRSANLTGISSVD |
| | | NLMISDAVHGVFMEVNEEGTEATGSTGAIGNIKHSLELEE |
| | | FRADHPFLFFIRYNPTNAILFFGRYWSP* |
| β-ovomucin | SEQ ID NO: 280 | CSTWGGGHFSTFDKYQYDFTGTCNYIFATVCDESSPDFNI |
| | | QFRRGLDKKIARIIIELGPSVIIVEKDSISVRSVGVIKLP |
| | | YASNGIQIAPYGRSVRLVAKLMEMELVVMWNNEDYLMVLT |
| | | EKKYMGKTCGMCGNYDGYELNDFVSEGKLLDTYKFAALQK |
| | | MDDPSEICLSEEISIPAIPHKKYAVICSQLLNLVSPTCSV |
| | | PKDGFVTRCQLDMQDCSEPGQKNCTCSTLSEYSRQCAMSH |
| | | QVVFNWRTENFCSVGKCSANQIYEECGSPCIKTCSNPEYS |
| | | CSSHCTYGCFCPEGTVLDDISKNRTCVHLEQCPCTLNGET |
| | | YAPGDTMKAACRTCKCTMGQWNCKELPCPGRCSLEGGSFV |
| | | TTFDSRSYRFHGVCTYILMKSSSLPHNGTLMAIYEKSGYS |
| | | HSETSLSAIIYLSTKDKIVISQNELLTDDDELKRLPYKSG |
| | | DITIFKQSSMFIQMHTEFGLELVVQTSPVFQAYVKVSAQF |
| | | QGRTLGLCGNYNGDTTDDFMTSMDITEGTASLFVDSWRAG |
| | | NCLPAMERETDPCALSQLNKISAETHCSILTKKGTVFETC |
| | | HAVVNPTPFYKRCVYQACNYEETFPYICSALGSYARTCSS |
| | | MGLILENWRNSMDNCTITCTGNQTFSYNTQACERTCLSLS |
| | | NPTLECHPTDIPIEGCNCPKGMYLNHKNECVRKSHCPCYL |
| | | EDRKYILPDQSTMTGGITCYCVNGRLSCTGKLQNPAESCK |
| | | APKKYISCSDSLENKYGATCAPTCQMLATGIECIPTKCES |
| | | GCVCADGLYENLDGRCVPPEECPCEYGGLSYGKGEQIQTE |
| | | CEICTCRKGKWKCVQKSRCSSTCNLYGEGHITTFDGQRFV |
| | | FDGNCEYILAMDGCNVNRPLSSFKIVTENVICGKSGVTCS |
| | | RSISIYLGNLTIILRDETYSISGKNLQVKYNVKKNALHLM |
| | | FDIIIPGKYNMTLIWNKHMNFFIKISRETQETICGLCGNY |
| | | NGNMKDDFETRSKYVASNELEFVNSWKENPLCGDVYFVVD |
| | | PCSKNPYRKAWAEKTCSIINSQVFSACHNKVNRMPYYEAC |
| | | VRDSCGCDIGGDCECMCDAIAVYAMACLDKGICIDWRTPE |
| | | FCPVYCEYYNSHRKTGSGGAYSYGSSVNCTWHYRPCNCPN |
| | | QYYKYVNIEGCYNCSHDEYFDYEKEKCMPCAMQPTSVTLP |
| | | TATQPTSPSTSSASTVLTETTNPPV* |
| Lysozyme | SEQ ID NO: 281 | KVFGRCELAAAMKRHGLDNYRGYSLGNWVCAAKFESNENT |
| | | QATNRNTDGSTDYGILQINSRWWCNDGRTPGSRNLCNIPC |
| | | SALLSSDITASVNCAKKIVSDGNGMNAWVAWRNRCKGTDV |
| | | QAWIRGCRL* |
| Lysozyme | SEQ ID NO: 282 | KVFGRCELAAAMKRHGLDNYRGYSLGNWVCVAKFESNFNT |
| | | QATNRNTDGSTDYGILQINSRWWCNDGRTPGSRNLCNIPC |
| | | SALLSSDITASVNCAKKIVSDGNGMSAWVAWRNRCKGTDV |
| | | QAWIRGCRL* |
| Lysozyme C (Human) | SEQ ID NO: 283 | KVFERCELARTLKRLGMDGYRGISLANWMCLAKWESGYNT |
| | | RATNYNAGDRSTDYGIFQINSRYWCNDGKTPGAVNACHLS |
| | | CSALLQDNIADAVACAKRVVRDPQGIRAWVAWRNRCQNRD |
| | | VRQYVQGCGV* |
| Lysozyme C (Bos | SEQ ID NO: 284 | KVFERCELARTLKKLGLDGYKGVSLANWLCLTKWESSYNT |
| taurus) | | KATNYNPSSESTDYGIFQINSKWWCNDGKTPNAVDGCHVS |
| | | CRELMENDIAKAVACAKHIVSEQGITAWVAWKSHCRDHDV |
| | | SSYVEGCTL* |
| Ovoinhibitor | SEQ ID NO: 285 | IEVNCSLYASGIGKDGTSWVACPRNLKPVCGTDGSTYSNE |
| | | CGICLYNREHGANVEKEYDGECRPKHVMIDCSPYLQVVRD |
| | | GNTMVACPRILKPVCGSDSFTYDNECGICAYNAEHHTNIS |
| | | KLHDGECKLEIGSVDCSKYPSTVSKDGRTLVACPRILSPV |
| | | CGTDGFTYDNECGICAHNAEQRTHVSKKHDGKCRQEIPEI |
| | | DCDQYPTRKTTGGKLLVRCPRILLPVCGTDGFTYDNECGI |
| | | CAHNAQHGTEVKKSHDGRCKERSTPLDCTQYLSNTQNGEA |
| | | ITACPFILQEVCGTDGVTYSNDCSLCAHNIELGTSVAKKH |
| | | DGRCREEVPELDCSKYKTSTLKDGRQVVACTMIYDPVCAT |
| | | NGVTYASECTLCAHNLEQRTNLGKRKNGRCEEDITKEHCR |
| | | EFQKVSPICTMEYVPHCGSDGVTYSNRCFFCNAYVQSNRT |
| | | LNLVSMAAC* |
| Cystatin | SEQ ID NO: 286 | MAGARGCVVLLAAALMLVGAVLGSEDRSRLLGAPVPVDEN |
| | | DEGLQRALQFAMAEYNRASNDKYSSRVVRVISAKRQLVSG |
| | | IKYILQVEIGRTTCPKSSGDLQSCEFHDEPEMAKYTTCTF |
| | | VVYSIPWLNQIKLLESKCQ* |
| Porcine Lipase | SEQ ID NO: 287 | SEVCFPRLGCFSDDAPWAGIVQRPLKILPWSPKDVDTRFL |
| | | LYTNQNQNNYQELVADPSTITNSNFRMDRKTRFIIHGFID |
| | | KGEEDWLSNICKNLFKVESVNCICVDWKGGSRTGYTQASQ |
| | | NIRIVGAEVAYFVEVLKSSLGYSPSNVHVIGHSLGSHAAG |
| | | EAGRRTNGTIERITGLDPAEPCFQGTPELVRLDPSDAKFV |
| | | DVIHTDAAPIIPNLGFGMSQTVGHLDFFPNGGKQMPGCQK |
| | | NILSQIVDIDGIWEGTRDFVACNHLRSYKYYADSILNPDG |
| | | FAGFPCDSYNVFTANKCFPCPSEGCPQMGHYADRFPGKTN |
| | | GVSQVFYLNTGDASNFARWRYKVSVTLSGKKVTGHILVSL |
| | | FGNEGNSRQYEIYKGTLQPDNTHSDEFDSDVEVGDLQKVK |
| | | FIWYNNNVINPTLPRVGASKITVERNDGKVYDFCSQETVR |
| | | EEVLLTLNPC* |
| Kid Lipase | SEQ ID NO: 288 | GLVAADRITGGKDFRDIESKFALRTPEDTAEDTCHLIPGV |
| | | TESVANCHFNHSSKTFVVIHGWTVTGMYESWVPKLVAALY |
| | | KREPDSNVIVVDWLSRAQQHYPVSAGYTKLVGQDVAKFMN |
| | | WMADEFNYPLGNVHLLGYSLGAHAAGIAGSLTSKKVNRIT |
| | | GLDPAGPNFEYAEAPSRLSPDDADFVDVLHTFTRGSPGRS |
| | | IGIQKPVGHVDIYPNGGTFQPGCNIGEALRVIAERGLGDV |
| | | DQLVKCSHERSVHLFIDSLLNEENPSKAYRCNSKEAFEKG |
| | | LCLSCRKNRCNNMGYEINKVRAKRSSKMYLKTRSQMPYKV |
| | | FHYQVKIHFSGTESNTYTNQAFEISLYGTVAESENIPFTL |
| | | PEVSTNKTYSFLLYTEVDIGELLMLKLKWISDSYFSWSNW |
| | | WSSPGFDIGKIRVKAGETQKKVIFCSREKMSYLQKGKSPV |
| | | IFVKCHDKSLNRKSG* |
| Porcine Lactoferrin | SEQ ID NO: 289 | APKKGVRWCVISTAEYSKCRQWQSKIRRTNPMFCIRRASP |
| | | TDCIRAIAAKRADAVTLDGGLVFEADQYKLRPVAAEIYGT |
| | | EENPQTYYYAVAVVKKGFNFQLNQLQGRKSCHTGLGRSAG |
| | | WNIPIGLLRRFLDWAGPPEPLQKAVAKFFSQSCVPCADGN |
| | | AYPNLCQLCIGKGKDKCACSSQEPYFGYSGAFNCLHKGIG |
| | | DVAFVKESTVFENLPQKADRDKYELLCPDNTRKPVEAFRE |
| | | CHLARVPSHAVVARSVNGKENSIWELLYQSQKKFGKSNPQ |
| | | EFQLFGSPGQQKDLLFRDATIGFLKIPSKIDSKLYLGLPY |
| | | LTAIQGLRETAAEVEARQAKVVWCAVGPEELRKCRQWSSQ |
| | | SSQNLNCS |
| | | LASTTEDCIVQVLKGEADAMSLDGGFIYTAGKCGLVPVLA |
| | | ENQKSRQSSSSDCVHRPTQGYFAVAVVRKANGGITWNSVR |
| | | GTKSCHTAVDRTAGWNIPMGLLVNQTGSCKFDEFFSQSCA |
| | | PGSQPGSNLCALCVGNDQGVDKCVPNSNERYYGYTGAFRC |
| | | LAENAGDVAFVKDVTVLDNTNGQNTEEWARELRSDDFELL |
| | | CLDGTRKPVTEAQNCHLAVAPSHAVVSRKEKAAQVEQVLL |
| | | TEQAQFGRYGKDCPDKFCLFRSETKNLLFNDNTEVLAQLQ |
| | | GKTTYEKYLGSEYVTAIANLKQCSVSPLLEACAFMMR* |
| Bovine Lactoferrin | SEQ ID NO: 290 | APRKNVRWCTISQPEWFKCRRWQWRMKKLGAPSITCVRRA |
| | | FALECIRAIAEKKADAVTLDGGMVFEAGRDPYKLRPVAAE |
| | | IYGTKESPQTHYYAVAVVKKGSNFQLDQLQGRKSCHTGLG |
| | | RSAGWIIPMGILRPYLSWTESLEPLQGAVAKFFSASCVPC |
| | | IDRQAYPNLCQLCKGEGENQCACSSREPYFGYSGAFKCLQ |
| | | DGAGDVAFVKETTVFENLPEKADRDQYELLCLNNSRAPVD |
| | | AFKECHLAQVPSHAVVARSVDGKEDLIWKLLSKAQEKFGK |
| | | NKSRSFQLFGSPPGQRDLLFKDSALGFLRIPSKVDSALYL |
| | | GSRYLTTLKNLRETAEEVKARYTRVVWCAVGPEEQKKCQQ |
| | | WSQQSGQNVTCATASTTDDCIVLVLKGEADALNLDGGYIY |
| | | TAGKCGLVPVLAENRKSSKHSSLDCVLRPTEGYLAVAVVK |
| | | KANEGLTWNSLKDKKSCHTAVDRTAGWNIPMGLIVNQTGS |
| | | CAFDEFFSQSCAPGADPKSRLCALCAGDDQGLDKCVPNSK |
| | | EKYYGYTGAFRCLAEDVGDVAFVKNDTVWENTNGESTADW |
| | | AKNLNREDFRLLCLDGTRKPVTEAQSCHLAVAPNHAVVSR |
| | | SDRAAHVKQVLLHQQALFGKNGKNCPDKFCLFKSETKNLL |
| | | FNDNTECLAKLGGRPTYEEYLGTEYVTAIANLKKCSTSPL |
| | | LEACAFLTR* |
| Saccharomyces | SEQ ID NO: 291 | APVNTTTEDETAQIPAEAVIGYSDLEGDFDVAVLPFSNST |
| cerevisiae | | NNGLLFINTTIASIAAKEEGVSLDKR |
| a-mating | | |
| factor signal peptide | | |
| and secretion signal | | |
| Saccharomyces | SEQ ID NO: 292 | APVNTTTEDETAQIPAEAVIGYSDLEGDFDVAVLPFSNST |
| cerevisiae | | NNGLLFINTTIASIAAKEEGVSLDKREAEA |
| a-mating | | |
| factor signal peptide | | |
| and secretion signal | | |
| ending with EAEA | | |
| EndoH- | SEQ ID NO: 293 | MTIAHHCIFLVILAFLALINVASGAPAPVKQGPTSVAYVE |
| Saccharomyces | | VNNNSMLNVGKYTLADGGGNAFDVAVIFAANINYDTGTKT |
| cerevisiae | | AYLHFNENVQRVLDNAVTQIRPLQQQGIKVLLSVLGNHQG |
| Flo5 fusion | | AGFANFPSQQAASAFAKQLSDAVAKYGLDGVDFDDEYAEY |
| (full ORF, including | | GNNGTAQPNDSSFVHLVTALRANMPDKIISLYNIGPAASR |
| peptides that are | | LSYGGVDVSDKFDYAWNPYYGTWQVPGIALPKAQLSPAAV |
| cleaved off post- | | EIGRTSRSTVADLARRTVDEGYGVYLTYNLDGGDRTADVS |
| translationally) | | AFTRELYGSEAVRTPGSSGSSGSSGSSGSSGSSGSSGSSE |
| | | AAAREAAAREAAAREAAARGGGGSGGGGSGGGGSATEACL |
| | | PAGQRKSGMNINFYQYSLKDSSTYSNAAYMAYGYASKTKL |
| | | GSVGGQTDISIDYNIPCVSSSGTFPCPQEDSYGNWGCKGM |
| | | GACSNSQGIAYWSTDLFGFYTTPTNVTLEMTGYFLPPQTG |
| | | SYTFSFATVDDSAILSVGGSIAFECCAQEQPPITSTNFTI |
| | | NGIKPW |
| | | DGSLPDNITGTVYMYAGYYYPLKVVYSNAVSWGTLPISVE |
| | | LPDGTTVSDNFEGYVYSFDDDLSQSNCTIPDPSIHTTSTI |
| | | TTTTEPWTGTFTSTSTEMTTITDTNGQLTDETVIVIRTPT |
| | | TASTITTTTEPWTGTFTSTSTEMTTVTGTNGQPTDETVIV |
| | | IRTPTSEGLITTTTEPWTGTFTSTSTEMTTVTGTNGQPTD |
| | | ETVIVIRTPTSEGLITTTTEPWTGTFTSTSTEVTTITGTN |
| | | GQPTDETVIVIRTPTSEGLITTTTEPWTGTFTSTSTEMTT |
| | | VTGTNGQPTDETVIVIRTPTSEGLISTTTEPWTGTFTSTS |
| | | TEVTTITGTNGQPTDETVIVIRTPTSEGLITTTTEPWTGT |
| | | FTSTSTEMTTVTGTNGQPTDETVIVIRTPTSEGLITRTTE |
| | | PWTGTFTSTSTEVTTITGTNGQPTDETVIVIRTPTTAISS |
| | | SLSSSSGQITSSITSSRPIITPFYPSNGTSVISSSVISSS |
| | | VTSSLVTSSSFISSSVISSSTTTSTSIFSESSTSSVIPTS |
| | | SSTSGSSESKTSSASSSSSSSSISSESPKSPTNSSSSLPP |
| | | VTSATTGQETASSLPPATTTKTSEQTTLVTVTSCESHVCT |
| | | ESISSAIVSTATVTVSGVTTEYTTWCPISTTETTKQTKGT |
| | | TEQTKGTTEQTTETTKQTTVVTISSCESDICSKTASPAIV |
| | | STSTATINGVTTEYTTWCPISTTESKQQTTLVTVTSCESG |
| | | VCSETTSPAIVSTATATVNDVVTVYPTWRPQTTNEQSVSS |
| | | KMNSATSETTTNTGAAETKTAVTSSLSRFNHAETQTASAT |
| | | DVIGHSSSVVSVSETGNTMSLTSSGLSTMSQQPRSTPASS |
| | | MVGSSTASLEISTYAGSANSLLAGSGLSVFIASLLLAII |
| A flexible GS linker | SEQ ID NO: 294 | GSSGSSGSSGSSGSSGSSGSSGSS |
| with higher S content | | |
| A flexible GS linker | SEQ ID NO: 295 | GGGGSGGGGSGGGGS |
| with much higher G | | |
| content | | |

1. An engineered eukaryotic cell comprising a surface displayed catalytic domain of an endoglycosidase, wherein the surface displayed catalytic domain of an endoglycosidase is a portion of a fusion protein expressed by the cell.

2. The engineered eukaryotic cell of claim 2, wherein the fusion protein further comprises an anchoring domain of a cell surface protein.

3. The engineered eukaryotic cell of claim 1, wherein the fusion protein comprises a portion of the endoglycosidase in addition to its catalytic domain.

4. The engineered eukaryotic cell of claim 1, wherein the fusion protein comprises substantially the entire amino acid sequence of the endoglycosidase.

5. The engineered eukaryotic cell of claim 1, wherein the endoglycosidase is endoglycosidase H.

6. The engineered eukaryotic cell of claim 1, wherein the fusion protein comprises an amino acid sequence that is at least 95%, 96%, 97%, 98%, 99%, or 100% identical to SEQ ID NO: 1 or SEQ ID NO:2.

7. The engineered eukaryotic cell of claim 1, wherein the fusion protein comprises a portion of the cell surface protein in addition to its anchoring domain.

8. The engineered eukaryotic cell of claim 1, wherein the fusion protein comprises substantially the entire amino acid sequence of the cell surface protein.

9. The engineered eukaryotic cell of claim 1, wherein the cell surface protein is selected from Sed1p, Flo5-2, or Flo11.

10. The engineered eukaryotic cell of claim 1, wherein the fusion protein comprises an amino acid sequence that is at least 95% identical to one of SEQ ID NO: 3 to SEQ ID NO: 7 and SEQ ID NO: 20.

11. The engineered eukaryotic cell of claim 1, wherein the anchoring domain stably attaches the fusion protein to the extracellular surface of the cell.

12. The engineered eukaryotic cell of claim 1, wherein upon translation the fusion protein comprises a signal peptide and/or a secretory signal.

13. The engineered eukaryotic cell of claim 1, wherein the anchoring domain is N-terminal to the catalytic domain in the fusion protein.

14. The engineered eukaryotic cell of claim 13, wherein the fusion protein comprises a linker C-terminal to the anchoring domain.

15. The engineered eukaryotic cell of claim 1, wherein the anchoring domain is C-terminal to the catalytic domain in the fusion protein.

16. The engineered eukaryotic cell of claim 15, wherein the fusion protein comprises a linker N-terminal to the anchoring domain.

17. The engineered eukaryotic cell of claim 1, wherein the cell surface protein is Sed1p and the endoglycosidase is endoglycosidase H.

18. The engineered eukaryotic cell of claim 17, wherein the fusion protein comprises an amino acid sequence that is at least 95% identical to SEQ ID NO: 9 or SEQ ID NO:

19. The engineered eukaryotic cell of claim 1, wherein the cell surface protein is Flo5-2 or Flo11 and the endoglycosidase is endoglycosidase H.

20. The engineered eukaryotic cell of claim 19, wherein the fusion protein comprises an amino acid sequence that is at least 95% identical to SEQ ID NO: 11 or SEQ ID NO: 12.

21. The engineered eukaryotic cell of claim 19, wherein the fusion protein comprises an amino acid sequence that is at least 95% identical to SEQ ID NO: 13 or SEQ ID NO: 14.

22. The engineered eukaryotic cell of claim 1, wherein the engineered eukaryotic cell comprises a mutation in its AOX1 gene and/or its AOX2 gene.

23. The engineered eukaryotic cell of claim 1, wherein the engineered eukaryotic cell is a yeast cell or a Pichia species.

24. The engineered eukaryotic cell of claim 23, wherein the yeast cell is a Pichia species.

25. The engineered eukaryotic cell of claim 1, further comprising a genomic modification that overexpresses a secretory glycoprotein.

26. The engineered eukaryotic cell of claim 25, wherein the secretory glycoprotein is an animal protein, e.g., an egg protein.

27. The engineered eukaryotic cell of claim 26, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

28. The engineered eukaryotic cell of claim 1, wherein the cell lacks a genomic modification that overexpresses a secretory glycoprotein.

29. The engineered eukaryotic cell of claim 1, comprising a nucleic acid sequence that encodes the fusion protein.

30. The engineered eukaryotic cell of claim 29, wherein the nucleic acid sequence that encodes the fusion protein is integrated into the cell's genome.

31. The engineered eukaryotic cell of claim 29, wherein the nucleic acid sequence that encodes the fusion protein is extrachromosomal.

32. The engineered eukaryotic cell of claim 29, wherein the nucleic acid sequence comprises an inducible promoter.

33. The engineered eukaryotic cell of claim 32, wherein the inducible promoter is an AOX1, DAK2, PEX11, FLD1, FGH1, DAS2, CAT1, MDH3, HAC1, BiP, RAD30, RVS161-2, MPP10, THP3, or GBP2 promoter.

34. The engineered eukaryotic cell of claim 29, wherein the nucleic acid sequence comprises an AOX1, TDH3, RPS25A, or RPL2A terminator.

35. The engineered eukaryotic cell of claim 29, wherein the nucleic acid sequence encodes a signal peptide and/or a secretory signal.

36. The engineered eukaryotic cell of claim 29, wherein the nucleic acid sequence comprises codons that are optimized for the species of the engineered cell.

37. A method for deglycosylating a secreted glycoprotein, the method comprising contacting a secreted protein with a fusion protein anchored to an engineered eukaryotic cell of claim 1, thereby providing a deglycosylated secreted glycoprotein.

38. The method of claim 37, wherein the secreted glycoprotein is expressed by the engineered eukaryotic cell.

39. The method of claim 37, wherein the fusion protein anchored to an engineered eukaryotic cell is more effective at deglycosylating the secreted protein than an intracellular endoglycosidase.

40. The method of claim 39, wherein the intracellular endoglycosidase is located within a Golgi vesicle.

41. The method of claim 39, wherein the intracellular endoglycosidase is linked to a membrane associating domain.

42. The method of claim 41, wherein the membrane associating domain comprises an amino acid sequence of OCH1.

43. The method of claim 37, wherein the secreted protein is expressed by a cell other than the engineered eukaryotic cell.

44. The method of claim 37, further comprising a step of isolating the deglycosylated secreted protein.

45. The method of claim 44, further comprising a step of drying the deglycosylated secreted protein.

46. The method of claim 37, wherein the secreted protein is an animal protein, e.g., an egg protein.

47. The method of claim 46, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

48. A method for deglycosylating a plurality of secreted glycoproteins, the method comprising contacting the plurality of secreted glycoproteins with a population of engineered eukaryotic cells of claim 1, thereby providing a plurality of deglycosylated secreted glycoproteins.

49. The method of claim 48, wherein substantially every secreted glycoprotein in the plurality of secreted proteins is deglycosylated upon contact with the population of engineered eukaryotic cells.

50. The method of claim 48, wherein the amount of deglycosylation of the secreted glycoproteins is not increased by further contacting the secreted protein with an isolated endoglycosidase.

51. The method of claim 48, wherein the amount of deglycosylation of the secreted glycoproteins is more than the amount obtained from a population of cells that express an intracellular endoglycosidase.

52. The method of claim 48, further comprising a step of isolating the plurality of deglycosylated secreted proteins.

53. The method of claim 52, further comprising a step of drying the plurality of deglycosylated secreted proteins.

54. The method of claim 48, wherein the secreted protein is an animal protein, e.g., an egg protein.

55. The method of claim 54, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

56. A method for expressing a fusion protein comprising an anchoring domain of a cell surface protein and a catalytic domain of an endoglycosidase, the method comprising obtaining the engineered eukaryotic cell of claim 1 and culturing the engineered eukaryotic cell under conditions that promote expression of the fusion protein.

57. The method of claim 56, wherein when the engineered eukaryotic cell comprises a nucleic acid sequence that encodes the fusion protein and comprises an inducible promoter, culturing the engineered eukaryotic cell under conditions that promote expression of the fusion protein comprises contacting the cell with an agent that activates the inducible promoter.

58. The method of claim 57, wherein the inducible promoter is an AOX1, DAK2, PEX11 promoter and the agent that activates the inducible promoter is methanol.

59. A population of engineered eukaryotic cells of claim 1.

60. A bioreactor comprising the population of engineered eukaryotic cells of claim 59.

61. A composition comprising an engineered eukaryotic cell of claim 1 and a secreted glycoprotein.

62. The composition of claim 61, wherein the secreted glycoprotein is an animal protein, e.g., an egg protein.

63. The composition of claim 62, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

64. A composition comprising an engineered eukaryotic cell of claim 1, a secreted protein that has been deglycosylated, and one or more oligosaccharides cleaved from the secreted protein.

65. The composition of claim 64, wherein the secreted glycoprotein is an animal protein, e.g., egg protein.

66. The composition of claim 65, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

67. An engineered eukaryotic cell which expresses a surface displayed catalytic domain of endoglycosidase H, wherein the catalytic domain is directly or indirectly tethered to the exterior surface of the cell.
